(19) United States
(12) Patent Application Publication
(10) Pub. No.: US 2023/0323384 A1
(43) Pub. Date: Oct. 12, 2023

(54) PLANTS HAVING A MODIFIED LAZY PROTEIN

(71) Applicant: University of Leeds, Leeds (GB)

(72) Inventors: Stefan Samuel KEPINSKI, Leeds (GB); Ryan Andrew Samuel KAYE, Leeds (GB)

(21) Appl. No.: 17/765,596

(22) PCT Filed: Oct. 1, 2020
(86) PCT No.: PCT/GB2020/052401

§ 371 Date: Mar. 31, 2022

(30) Foreign Application Priority Data
Oct. 1, 2019 (GB) 1914137.3

(51) Int. Cl. C12N 15/82; C07K 14/415; C12Q 1/6895

(52) U.S. Cl. CPC C12N 15/8273; C07K 14/415; C12N 2310/20; C12Q 2600/13; C12Q 2600/156; C12Q 1/6895

(57) ABSTRACT

The invention relates to genetically altered plants with improved traits, in particular steeper root growth. The invention also relates to methods for making such plants and methods for modulating root growth, in particular methods that employ gene editing techniques.

### INTRODUCTION

Soil resource acquisition is a primary limitation to crop production. In poor nations drought and low soil fertility cause low yields and food insecurity, while in rich nations irrigation and intensive fertilization cause environmental pollution and resource degradation. The optimisation of root system architecture and function is recognised to be a critical component of crop improvement for the sustainable intensification of agriculture, and in particular the pressing need to reduce environmentally damaging agricultural inputs. The development of new crop cultivars with enhanced soil resource acquisition is therefore an important strategic goal for global agriculture. Amongst root traits, steep rooting angle is a high value breeding target associated with improved performance of crops at lower levels of nitrate fertiliser application and irrigation.

Root systems are central to the acquisition of water and nutrients by plants and have thus become a focus of plant breeders and seed companies. In particular, traits such as root length, branching and growth angle determine the distribution of root surface area within the soil profile where nutrients and water are unevenly distributed. For example, nitrogen (in the form of nitrate) and water are highly mobile within the soil and levels are generally higher within the deeper layers of the soil (Lynch 2013 Ann. Bot. 112:347-357).

Crop root systems are unable to completely exploit available soil resources; this is especially true of annual crops, which require time to develop extensive root systems, during which time soil resources may be lost to evaporation (including denitrification), leaching, soil fixation into unavailable forms, or competing organisms. Deep rooting offers many advantages to plants, including greater mechanical stability and greater acquisition of resources such as nutrients and water during crucial growth stages, including under water and nutrient deficit conditions, thereby helping plants to attain greater biomass production and yield than shallow-rooted plants. This can be advantageous compared to lateral growth of shallow-rooted plants which have fewer roots distributed into deeper soil areas. In particular, when plants with deeper roots are exposed to drought, they are able to absorb water from deeper soil areas.

Root growth angle, which affects how deeply roots penetrate into the soil, is regulated by multiple genes, as well as by environmental factors and plant growth stages. The LAZY family of genes have been described in Arabidopsis and rice, these are known to have some control over both root and shoot growth angle (Yoshihara et al, LAZY Genes Mediate the Effects of Gravity on Auxin Gradients and Plant Architecture. Plant Physiol. 2017 Oct.; 175(2):959-969; Guseman et al, DRO1 influences root system architecture in Arabidopsis and Prunus species. Plant J. 2017 Mar.; 89(6):1093-1105). A rice (Oryza sativa) mutant led to the discovery of a plant-specific LAZY1 protein that controls the orientation of shoots. Arabidopsis (Arabidopsis thaliana) possesses six LAZY genes having spatially distinct expression patterns. It has been proposed that AtLAZY proteins control plant architecture by coupling gravity sensing to the formation of auxin gradients that override a LAZY-independent mechanism that creates an opposing gravity-induced auxin gradient (Yoshihara et al, supra).

A knock out mutation of AtDRO1, also known as AtLAZY4, led to more horizontal (shallow) lateral root angles. Overexpression of AtDRO1 under a constitutive promoter resulted in steeper lateral root angles, as well as shoot phenotypes including upward leaf curling, shortened siliques and narrow lateral branch angles. A conserved C-terminal EAR-like motif found in IGT genes was required for these ectopic phenotypes (Guseman et al, supra).

In rice, DEEPER ROOTING 1 (DRO1) controls the gravitropic response of root growth angle. DRO1 was isolated as a functional allele that controls the gravitropic curvature of rice roots. This gene was identified in the deep-rooting cultivar Kinandang Patong (a traditional tropical japonica upland cultivar from the Philippines) and originated in the genetic background of the shallow rooting parent cultivar IR64, which is a modern lowland indica cultivar that is widely grown in South and South-east Asia. DRO1 plays a significant role in the acquisition of resources that permit higher yield. IR64-type Dro1 is a loss of function mutant and the function of Dro1 is impaired resulting in shallow rooting (Uga et al. Control of root system architecture by DEEPER ROOTING 1 increases rice yield under drought conditions. Nature Genetics, 45, 1097-1102, 2013; EP2518148).

An orthologue of rice DRO1 has also been identified in Prunus trees (PpeDRO1, US2018094272).

The present invention is aimed at providing alternative and improved plants and methods for manipulating plants to alter root growth. These plants have a deeper/steeper root architecture.

### SUMMARY

The inventors have identified a conserved motif in the protein encoded by LAZY4 gene family members, termed LAZY4D motif herein, and have shown that this conserved motif is involved in the regulation of root growth. Manipulation of amino acid sequence of this motif in plants enables the generation and identification/selection of new plants with an improved (deeper/steeper) root phenotype.

As explained below, the LAZY4D motif is a motif in the protein located in the middle of the AtLAZY4 protein sequence, far from the N- and C termini. As shown in FIG. 2, the LAZY4D motif is a small motif in the Arabidopsis LAZY4 protein that is highly conserved throughout higher plants. The motif is defined in SEQ ID NO. 3, 4, 5, 6 and 73. SEQ ID NO. 6 shows the full length consensus motif, SEQ ID NO. 5 shows the motif as in Arabidopsis and SEQ ID Nos. 73, 3 and 4 show highly conserved parts within the larger motif. Thus, the term LAZY4D motif as used herein refers to SEQ ID NO. 3, 4, 5, 6 and 73 unless otherwise specified. In one embodiment, the motif is as in SEQ ID NO. 6. In one embodiment, the motif is as in SEQ ID NO. 73. In one embodiment, the motif is as in SEQ ID NO. 5. In one embodiment, the motif is as in SEQ ID NO. 4. In another embodiment, the motif is as in SEQ ID NO. 3.

As explained above, LAZY genes have been identified in a number of plant species, including Arabidopsis thaliana and rice. It has also been shown that knock out mutations of LAZY/DRO genes as well as overexpression of these genes can affect root growth. However, the present inventors have identified a conserved motif in certain LAZY genes, which, if mutated, confers a dominant gain of function mutation that results in altered root growth; i.e. a steeper root angle. A single mutation is sufficient to confer the phenotype. This allows the targeted manipulation of LAZY homologues/orthologues in a crop plant to introduce the gain of function mutation and confer a beneficial phenotype. The mutation is dominant, avoiding the problems of gene redundancy and making for a simple, genome-editable technology for the re-engineering of root system architecture in existing, otherwise elite crop varieties.

The inventors have thus identified a single nucleotide mutation in the LAZY4 gene of Arabidopsis thaliana (Arabidopsis) that results in more vertical lateral root growth (see examples and FIG. 1A and B). The mutation has been named lazy4D because it is completely dominant: individuals heterozygous and homozygous for the mutant alleles are phenotypically indistinguishable.

The finding of the effects of the lazy4D mutation paves the way for a much more straightforward route to inducing steeper rooting in elite cultivars that in many cases have been bred for performance at relatively high fertiliser application rates. The dominant nature of the mutation offers significant advantages in polyploid crops where genetic redundancy can be a confounding issue and in species such as maize, where seeds are often supplied as F1 hybrids. Further, in Arabidopsis, the highest expression of LAZY4 is seen in the root (Yoshihara et al, supra) this is also true of the wheat orthologues, with little or no expression in aerial parts of the plant, making modification of LAZY4 an ideal target for altering the root architecture while avoiding possible deleterious effects on above-ground aspects for the crop such as shoot architecture and grain production.

The aspects of the invention exclude embodiments that are solely based on generating plants by traditional breeding methods.

Thus, in a first aspect, the invention relates to a genetically altered plant wherein said plant comprises a dominant gain of function mutation in a LAZY4 nucleic acid sequence encoding for a protein having a LAZY4D motif (i.e. SEQ ID NO. 3, 4, 5, 6 or 73).

The plant may comprise a mutation in a LAZY4 nucleic acid sequence encoding a mutant LAZY4 protein comprising a mutation in the LAZY4D motif (SEQ ID NO. 3, 4, 5, 6 or 73). For example, one or more amino acid residue in the LAZY4D motif (SEQ ID NO. 3, 4, 5, 6 or 73) is substituted with another amino acid residue. For example, said amino acid residue is R. For example, the LAZY4 nucleic acid sequence comprises SEQ ID NO. 1 or a homolog, orthologue or functional variant thereof. Said homolog or orthologue may be a LAZY4 nucleic acid sequence of a dicot or monocot plant, such as rice (Oryza sativa), maize (Zea mays), wheat (Triticum aestivum), sorghum (Sorghum bicolor, Sorghum vulgare), Brassica, soybean, cotton and millet. For example, the LAZY4 protein sequence is selected from SEQ ID NO. 7, 9, 11, 13, 15, 17, 19, 21, 23, 25, 27, 29, 31, 33, 35, 37, 39, 41, 43, 62, 64, 66, 67, 69 or 71 or a functional variant thereof. For example, the mutation is in the endogenous LAZY4 nucleic acid sequence. For example, the mutation is introduced using targeted genome modification. For example, said mutation is introduced using a rare-cutting endonuclease, for example a TALEN, ZFN or CRISPR/Cas9. The plant may have modulated root growth compared to a control plant.

In one embodiment, the plant is heterozygous or homozygous for the mutation.

The invention also relates to a method for modulating root growth in a plant comprising introducing a dominant gain of function mutation into a LAZY4 nucleic acid encoding for a protein having a LAZY4D motif (SEQ ID NO. 3, 4, 5, 6 or 73).

In another aspect, the invention relates to an isolated mutant LAZY4 nucleic acid sequence encoding a mutant LAZY4 protein comprising a dominant gain of function mutation.

In another aspect, the invention relates to a vector comprising an isolated nucleic acid described herein.

In another aspect, the invention relates to a host cell comprising a vector described herein.

In another aspect, the invention relates to a nucleic acid construct comprising a guide RNA that comprises a sequence selected from SEQ ID NOs. 45 to 60.

In another aspect, the invention relates to a plant comprising a nucleic construct comprising a guide RNA that comprises SEQ ID NOs. 45 to 60.

In another aspect, the invention relates to a method for producing a plant with modulated root growth, comprising introducing a dominant gain of function mutation into a LAZY4 nucleic acid having a LAZY4D motif (SEQ ID NO. 3, 4, 5, 6 or 73).

In another aspect, the invention relates to a method for identifying a plant with altered root growth compared to a control plant comprising detecting in a population of plants one or more polymorphisms in the LAZY4D motif of a LAZY4 nucleic acid sequence (SEQ ID NO. 1) wherein the control plant is homozygous for a LAZY4 nucleic acid that encodes a protein having a wild type LAZY4D motif (SEQ ID NO. 3, 4, 5, 6 or 73).

In another aspect, the invention relates to a detection kit for determining the presence or absence of a polymorphism in the LAZY4D motif (SEQ ID NO. 3, 4, 5, 6 or 73) encoded by a LAZY4 nucleic acid sequence in a plant.

### FIGURES

The invention is further described in the following non-limiting figures:

FIG. 1: Root angle phenotype of lazy4D and substituted amino acids at the same position. LAZY4D has a significantly more vertical lateral root angle than wt Col-0 (A and B). This is true for other amino acid substitutions at the lazy4D position (A and C), P<0.05 for all points. Scale bars represent 5 mm, error bars represent SEM.

FIG. 2: The LAZY4D motif. The motif containing the lazy4D mutation is conserved in LAZY2 and crop species including wheat, maize and soybean.

FIG. 3: Alternative mutations in the LAZY4D motif also change root angle. Ecotypes with a naturally occurring polymorphism that results in a V143A change in LAZY4D have a more vertical lateral root phenotype (P<0.05), error bars represent SEM.

FIG. 4: Replication of the LAZY4D mutation in the AtLAZY4 paralog AtLAZY2 also results in more vertical lateral roots. Site directed mutagenesis of the equivalent arginine (R143) in the AtLAZY4 paralog AtLAZY2 also results in significantly more vertical lateral roots than wt (A,C,D), this mutation is also dominant in nature as it is capable of overriding the native protein when the mutant is transformed into wt (A,D) p<0.05 for all points, Students T-test, n=10. There is no significant difference (A) between the lateral root angle of the construct transformed into wt Col-0 (C) and the lazy2 knockout line (D) p>0.05 at all points, Students T-test. All error bars represent SEM, scale bars represent 10 mm.

FIG. 5: Shows other mutations within the LAZY4D motif which also resulted in more vertical lateral roots. Site directed mutagenesis of C137, P138, V143, D144, R146, S139, L129, P130 or R133 in AtLAZY4 also results in significantly more vertical lateral roots than Wt (A) and the knockout mutant lazy4 (B), this mutation is also dominant in nature as it is capable of overriding the native protein when the mutant is transformed into Wt Col-0 (A), p<0.05 for all points, Students T-test, n=10. All error bars represent SEM.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

The invention relates to a genetically altered plant wherein said plant comprises a dominant gain of function mutation in a LAZY4 nucleic acid sequence. The invention also relates to methods for modulating root growth comprising introducing a dominant gain of function mutation into a LAZY4 nucleic acid.

In one embodiment, the mutation is in a LAZY4 nucleic acid sequence and results in a mutant LAZY4 protein comprising a mutation in the LAZY4D motif (SEQ ID NO. 3, 4, 5, 6 or 73).

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), naturally occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds. The term “allele” designates any of one or more alternative forms of a gene at a particular locus. Heterozygous alleles are two different alleles at the same locus. Homozygous alleles are two identical alleles at a particular locus. A wild type (wt) allele is a naturally occurring allele without a modification at the target locus.

The terms “increase”, “improve” or “enhance” are interchangeable. Yield or drought resistance for example can be increased by at least 3%, 4%, 5%, 6%, 7%, 8%, 9% or 10%, preferably at least 15% or 20%, more preferably 25%, 30%, 35%, 40% or 50% or more in comparison to a control plant. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield comprises one or more of and can be measured by assessing one or more of: increased seed yield per plant, increased seed filling rate, increased number of filled seeds, increased harvest index, increased number of seed capsules and/or pods, increased seed size, increased growth or increased branching, for example inflorescences with more branches. Yield is increased relative to control plants.

For the purposes of the invention, a “genetically altered plant” or “mutant plant” is a plant that has been genetically altered compared to a control plant.

A control plant as used herein is a plant, which has not been modified according to the methods of the invention. Accordingly, the control plant does not have a mutant lazy4D nucleic acid sequence as described herein. In one embodiment, the control plant is a wild type plant that does not have a gain of function mutation in a LAZY4 nucleic acid, for example does not have a modification at the nucleic acid encoding the LAZY4D motif. In another embodiment, the control plant is a plant that does not have a mutant lazy4D nucleic acid sequence nucleic acid sequence as described here, but is otherwise modified. The control plant is typically of the same plant species, preferably the same ecotype or the same or similar genetic background as the plant to be assessed.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, protoplasts, callus tissue, embryos, meristematic regions, gametophytes, sporophytes, pollen and microspores, again wherein each of the aforementioned comprises the gene/nucleic acid of interest.

Recently, genome editing techniques have emerged as alternative methods to conventional mutagenesis methods (such as physical and chemical mutagenesis) or methods using the expression of transgenes in plants to produce mutant plants with improved phenotypes that are important in agriculture. These techniques employ sequence-specific nucleases (SSNs) including zinc finger nucleases (ZFNs), transcription activator-like effector nucleases (TALENs), and the RNA-guided nuclease Cas9 (CRISPR/Cas9), which generate targeted DNA double-strand breaks (DSBs), which are then repaired mainly by either error-prone non-homologous end joining (NHEJ) or high-fidelity homologous recombination (HR). As explained in detail herein, mutations according to the invention can be introduced into plants using targeted genome modification based on such editing techniques.

For the purposes of certain other embodiments of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods.

The term “vector” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression of the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self-replicating extrachromosomal vectors or vectors which integrate into a host genome. Preferred vectors are those capable of autonomous replication and/or expression of nucleic acids to which they are linked. Vectors capable of directing the expression of genes to which they are operatively linked are referred to herein as “expression vectors”.

The term “regulatory sequences” is used interchangeably with “regulatory elements” herein refers to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequences to which they are operatively linked. Regulatory sequences often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

As used herein, a “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest. In one embodiment, the promoter is a constitutive promoter. A “constitutive promoter” refers to a promoter that is transcriptionally active during most, but not necessarily all, phases of growth and development and under most environmental conditions, in at least one cell, tissue or organ. Examples of constitutive promoters include but are not limited to actin, HMGP, CaMV19S, GOS2, rice cyclophilin, maize H3 histone, alfalfa H3 histone, 34S FMV, rubisco small subunit, OCS, SAD1, SAD2, nos, V-ATPase, super promoter, G-box proteins and synthetic promoters. In another aspect of the invention there is provided a vector comprising the nucleic acid sequence described above.

Plants of the invention have modified root phenotype, i.e. modified root growth compared to a control plant. The term modified root growth refers to a root growth with a steeper root angle compared to the root angle found in a control plant. The root growth angle is defined as the angle between the horizontal and the long axis of each root, and can be quantified to provide a synthetic indicator of the proportion of the total number of roots that grow in a primarily vertical direction. Plants of the invention have a significantly more vertical lateral root angle than control plants. This can be tested in various ways. For e.g. rice plants, root growth angle can be simply measured in a hydroponic system using a small basket at the young seedling stage (the “basket method”). For example, the root angle can be reduced by at least 5% or at least 10% resulting in a steeper root angle. As explained herein, steeper root growth can result in increased drought resistance and ultimately increased yield. For example, mild drought stress can be achieved by providing about 50% of the water needed to achieve maximum yield.

In a first aspect, the invention provides a genetically altered plant wherein said plant comprises a dominant gain of function mutation in a LAZY4 nucleic acid sequence having a LAZY4D motif (SEQ ID NO. 3, 4, 5, 6 or 73).

Examples of dominant gain of function mutations are described herein. However, any mutation that results in a dominant gain of function as described herein is encompassed within the scope of the invention. As used herein, “dominant” also encompasses “semi-dominant” or “partially dominant”. Therefore, the mutant allele may be fully dominant, partially dominant or semi-dominant. Preferably, the mutant allele is fully dominant.

According to the various aspects of the invention, a LAZY4 nucleic acid sequence is characterised by the presence of a LAZY4D motif (SEQ ID NO. 3, 4, 5, 6 or 73). Thus, as used herein, the term LAZY4 nucleic acid sequence or LAZY4 gene refers to a nucleic acid sequence, e.g. a gene, that encodes a protein characterised by the presence of the conserved LAZY4D motif (SEQ ID NO. 3, 4, 5, 6 or 73). The motif CPSSLEVDRR (SEQ ID NO. 4) can also be found in AtLAZY2. The inventors have shown that replication of the LAZY4D mutation in the AtLAZY4 paralog AtLAZY2 also results in more vertical lateral roots. Thus, the term LAZY4 nucleic acid sequence or LAZY4 gene refers to a nucleic acid sequence, e.g. a gene, that encodes a protein characterised by the presence of the conserved LAZY4D motif (i.e. SEQ ID NO. 3, 4, 5, 6 or 73) and this can be a homolog, paralog, orthologue or functional variant of AtLAZY4.

The inventors identified the LAZY4D motif in the AtLAZY4 gene. The locus of the AtLAZY4 gene (also termed AtDRO1, ATNGR2, DEEPER ROOTING 1, DRO1) is AT1G72490 (GenBank Accession NM_105908; Uniprot Q5XVG3-1). AtDRO1 is a member of the IGT gene family and is expressed in roots and involved in leaf and root architecture, specifically the orientation of lateral root angles. It is also involved in determining lateral root branch angle. The wild type gene sequence is shown as SEQ ID NO. 1 below. The wild type protein sequence is shown as SEQ ID NO. 2.

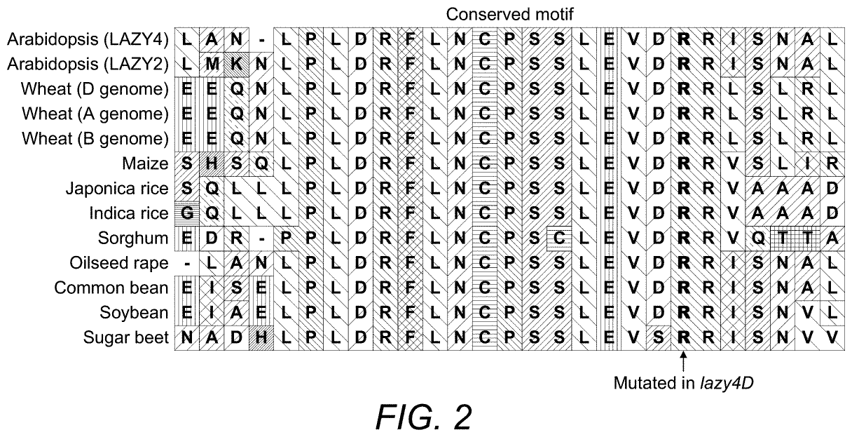

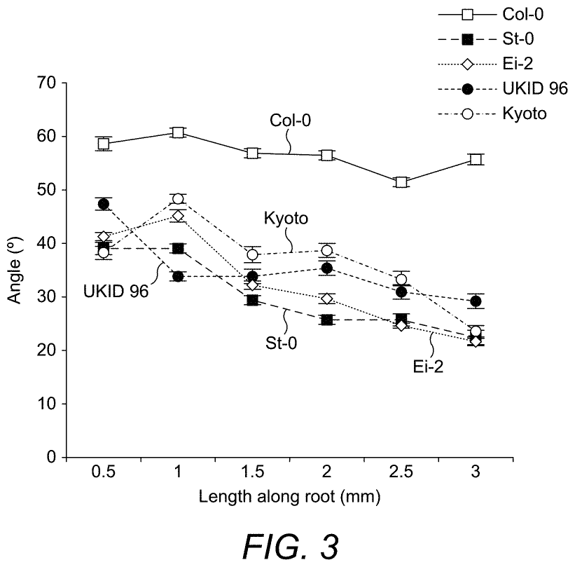

The LAZY4D motif is a motif in the protein located in the middle of the AtLAZY4 protein sequence, far from the N- and C termini. As shown in FIG. 2, the LAZY4D motif is a small motif in the Arabidopsis LAZY4 protein that is highly conserved throughout higher plants. The wild type, i.e. non-mutant, LAZY4D motif comprises the following residues: CPSXLEVDRR (SEQ ID NO. 3) wherein X is selected from S or C. In one embodiment, X is S and the LAZY4D motif has the following sequence: CPSSLEVDRR (SEQ ID NO. 4). In some embodiments, L in this sequence is replaced by F, for example in some Brassica species.

In one embodiment, the LAZY4D motif comprises or consists of the following residues: LANLPLDRFLNCPSSLEVDRRISNAL (SEQ ID NO. 5; the residues of the LAZY4D motif as discussed above are shown in bold) or a sequence with at least 60%, 75%, 80%, or 90% sequence identity thereto or a sequence with 1, 2 or 3 substitutions and which includes the conserved sequence CPSXLEVDRR (SEQ ID NO. 3), e.g. CPSSLEVDRR (SEQ ID NO. 4). In one embodiment, the LAZY4D motif comprises or consists of the following residues X₁X₁X₁X₂LPLDRFLNCPSXLEVDRRX₁X₁X₁X₁X₁ (SEQ ID NO. 6) wherein X₁ is any naturally occurring amino acid and X₂ is either present or absent and if present, is any naturally occurring amino acid. In one embodiment, the LAZY4D motif comprises or consists of the following residues: LPLDRFLNCPSXLEVDRR (SEQ ID NO. 73) wherein X is selected from S or C. A skilled person will appreciate that due to the degeneracy of codons, i.e. the redundancy of the genetic code, the part of the LAZY4 gene sequence that encodes the protein may vary between different LAZY4 homologs/orthologues. In some embodiment, L in the sequence LEVDR is replaced by F, for example in some Brassica species.

In another embodiment, LAZY4 family members also comprise the conserved protein motif IGT.

A LAZY4 nucleic acid can thus be identified by routine methods by determining the presence or absence of the LAZY4D motif.

The LAZY4D motif is different from the C-terminal motif mentioned by Guseman et al (2017, supra) and identified in AtDRO1. The motif identified by Guseman et al is located at the C terminus of AtDRO1. It is also worth noting that although they are considered homologues/orthologues of the rice gene DRO1, DRO1 bears little sequence similarity with AtDRO1 and the protein does not contain the LAZY4D motif. However, other orthologues in rice do have the LAZY4D motif (see FIG. 2).

According to one embodiment, the plant comprises a mutation in a LAZY4 nucleic acid sequence encoding a mutant LAZY4 protein comprising a mutation in the LAZY4D motif (e.g. SEQ ID NO. 3, 4, 5, 6 or 73, the wild type sequence is shown in SEQ ID NO. 3). Thus, according to the various aspects of the invention, the LAZY4 nucleic acid sequence is mutated compared to a control LAZY4 nucleic acid sequence, for example by targeted genome modification, thus encoding a mutant LAZY4 protein.

In one embodiment, one or more amino acid residue in the LAZY4D motif is substituted with another amino acid residue. In one embodiment, one or more of the following residues is substituted with another amino acid residue: C, P, S, S/C, L, E, V, D, R or R. In one embodiment, the residue mutated is the penultimate R in the motif. In one embodiment, the residue mutated is the last R in the motif. In one embodiment, the residue mutated is C, P, V, D, R, L or S (using the numbering in the Arabidopsis motif, these are residues C137, P138, V143, D144, R146, S139, L129, P130 and/or R133). Substitution can be with any suitable amino acid, for example A or G. In one embodiment, the substitution is as follows: C137A, P138A, V143A, D144A, R146A, S139A, L129A, P130A and/or R133A. A skilled person would understand that where there are differences in homologs, the equivalent residue in the homolog is mutated.

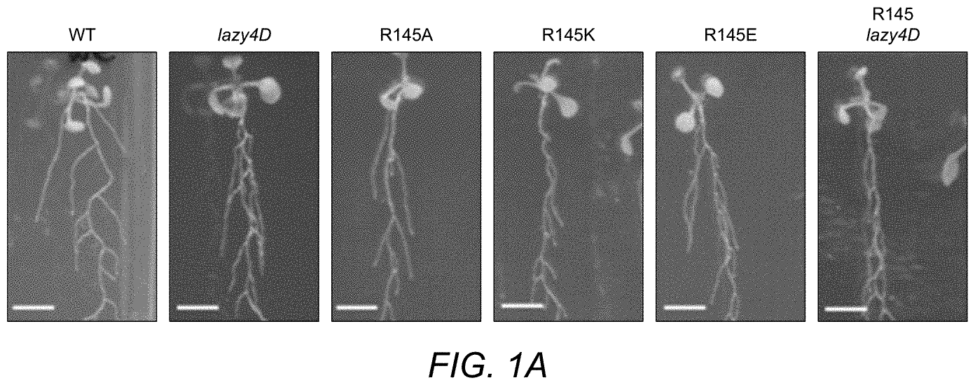

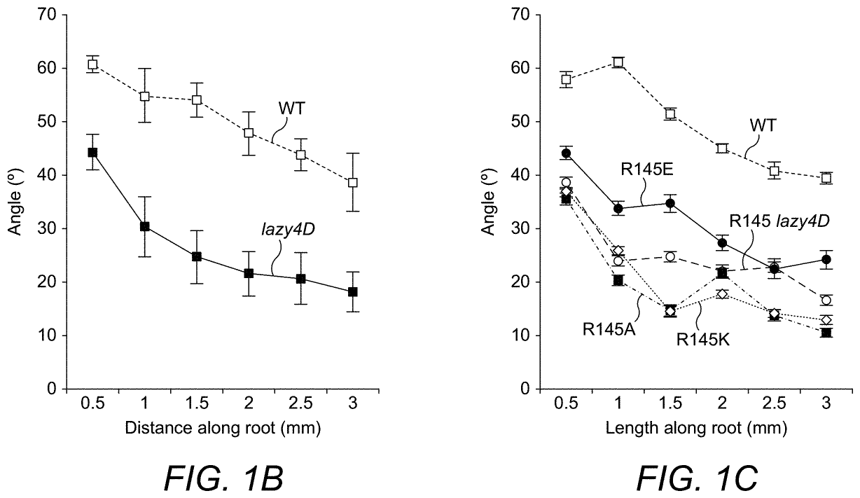

The inventors have shown that substitution of this penultimate R by a number of chemically-diverse amino acids results in the same dominant gain of function phenotype, indicating that it is loss of R rather than gain of another particular amino acid that is critical in inducing steeper root growth (FIG. 1A and C). Thus, the one or more amino acid residues in the LAZY4D motif, for example the penultimate R, can be substituted with any natural amino acid residue. In one embodiment, the target residue, for example the penultimate R, is substituted with a neutral amino acid residue, for example A or G or with W (for example when wheat is targeted).

In one embodiment, the (wild type) LAZY4 nucleic acid sequence comprises or consists of SEQ ID NO. 1 or a homolog, orthologue or functional variant thereof. This encodes a (wild type) LAZY4 protein comprising or consisting of SEQ ID NO. 2. As explained above, in one embodiment, the mutation resides in the conserved LAZY4D motif (e.g. SEQ ID NO. 3, 4, 5, 6, 73).

The term “functional variant of a nucleic acid sequence” as used herein with reference to SEQ ID NO: 1 refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. The term “functional variant of a amino acid sequence” as used herein with reference to SEQ ID NO: 2 refers to a variant protein sequence

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence; e.g. SEQ ID NO. 1 or a homolog or orthologue thereof.

The term homolog designates another LAZY4 gene from Arabidopsis characterised by the presence of the LAZY4D motif (e.g. SEQ ID NO. 3, 4, 5, 73 and/or 6). The term orthologue as used herein designates an AtLAZY4 gene orthologue from other plant species. A homolog or orthologue may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequence presented by SEQ ID NO: 1 or to the amino acid sequence shown in SEQ ID NO: 2. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, e.g. 90%, 91%, 92%, 93%, 94%, 95%, 96%, 97%, 98%, or at least 99%. Functional variants of LAZY4 homologs/orthologues as defined above are also within the scope of the invention. Examples are orthologues from crop species as listed below.

In one embodiment, the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66, 68, 70 or 72 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% thereto. All of these sequences encode a protein characterised by the presence of the LAZY4D motif as shown in one or more of SEQ ID NO. 3, 4, 5, 73 and/or 6. In one embodiment, the LAZY4 amino acid sequence is selected from SEQ ID NO. 7, 9, 11, 13, 15, 17, 19, 21, 23, 25, 27, 29, 31, 33, 35, 37, 39, 41, 43, 61, 63, 65, 67, 69, 71 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% thereto. All of these sequences are characterised by the presence of the LAZY4D motif as shown in one or more of SEQ ID NO. 3, 4, 5, 73 and/or 6.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologs/orthologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g. at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a variant as used herein can comprise a nucleic acid sequence encoding a LAZY4 polypeptide as defined herein that is capable of hybridising under stringent conditions as defined herein to a nucleic acid sequence as defined in SEQ ID NO: 1.

In one embodiment, the orthologue of the LAZY4 nucleic acid sequence as shown in SEQ ID NO. 1 is a LAZY4 nucleic acid of a dicot or monocot plant. Thus, the genetically altered plant may be a monocot or dicot plant with a mutation in an endogenous LAZY4 nucleic acid sequence encoding a mutant LAZY4 protein comprising a mutation in the LAZY4D motif (SEQ ID NO. 3, 4, 5, 6 or 73).

In one embodiment, the plant is a crop plant. By crop plant is meant any plant which is grown on a commercial scale for human or animal consumption or use. In one embodiment, the plant is a cereal. In another embodiment, the plant is selected from rice (Oryza sativa), maize (Zea mays), wheat (Triticum aestivum), sorghum (Sorghum bicolor, Sorghum vulgare), Brassica, soybean and millet. In one embodiment, the plant is selected from rice, such as the japonica or indica varieties. Other exemplary genetically altered plants of the invention include, but are not limited to, canola (Brassica napus, Brassica rapa ssp., Brassica oleracea), alfalfa (Medicago sativa), rape (Brassica napus), rye (Secale cereale), sunflower (Helianthus annuus), soybean (Glycine max), tobacco (Nicotiana tabacum), potato (Solanum tuberosum), peanuts (Arachis hypogaea), cotton (Gossypium hirsutum), sweet potato (Ipomoea batatas), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp), avocado (Persea americana), fig (Ficus carica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus domestica), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris), duckweed (Lemna), oats, barley, vegetables, ornamentals, conifers, and turfgrasses (e.g., for ornamental, recreational or forage purposes), Cannabis sativa, Cannabis indica, Pennycress (Thlaspi spp.) and biomass grasses (e.g., switchgrass and miscanthus).

In one embodiment, the plant is heterozygous or homozygous for the mutation.

The invention also extends to harvestable parts of a genetically altered plant of the invention as described above such as, but not limited to seeds, leaves, flowers, stems and roots. The invention furthermore relates to products derived, preferably directly derived, from a harvestable part of such a plant, such as dry pellets or powders, oil, fat and fatty acids, flour, starch or proteins. The invention also relates to food products and food supplements comprising the plant of the invention or parts thereof. In one aspect, the invention relates to a seed of a mutant plant of the invention.

In another embodiment, the present invention provides a regenerable mutant plant as described herein and cells for use in tissue culture. The tissue culture will preferably be capable of regenerating plants having essentially all of the physiological and morphological characteristics of the foregoing mutant plant, and of regenerating plants having substantially the same genotype. Preferably, the regenerable cells in such tissue cultures will be callus, protoplasts, meristematic cells, cotyledons, hypocotyl, leaves, pollen, embryos, roots, root tips, anthers, pistils, shoots, stems, petioles, flowers, and seeds. Still further, the present invention provides plants regenerated from the tissue cultures of the invention.

In one embodiment, the genetically altered plant is a plant that has been altered using a mutagenesis method, such as any of the mutagenesis methods described herein. In one embodiment, the mutagenesis method is targeted genome modification (genome editing) as further explained herein. Such plants have an altered root phenotype as described herein. Therefore, in this example, the phenotype is conferred by the presence of an altered plant genome, i.e., a mutated endogenous LAZY4 gene. In one embodiment, the LAZY4 gene sequence is specifically targeted using targeted genome modification. Thus, the presence of a mutated LAZY4 gene sequence is not conferred by the presence of transgenes expressed in the plant. In other words, the genetically altered plant can be described as transgene-free. Gene editing techniques that can be used to generate the plant are further described below.

In one embodiment, the genetically altered plant is not exclusively obtained by means of an essentially biological process. For example, the mutation has been introduced in the LAZY4 nucleic acid sequence using targeted genome modification, for example with a construct as described herein.

In yet another embodiment, the plant does not comprise a naturally occurring polymorphism in a LAZY4 gene which results in an amino acid substitution of an amino acid in the LAZY4D motif (SEQ ID NO. 3).

In one embodiment, the plant and/or the LAZY4 nucleic acid sequence is not Arabidopsis. In one embodiment, the plant and/or the LAZY4 nucleic acid sequence is not Arabidopsis and the mutation in the LAZY4 nucleic acid sequence does not result in a mutant protein which does not have a modification at V143 in the conserved LAZY4D motif (SEQ ID NO. 3,4, 5, 6 or 73)

In another embodiment, the genetically altered plant has been modified using transgenic approaches as further explained herein. For example, the plant may have been modified to overexpress a LAZY4 nucleic acid sequence with a dominant gain of function mutation, for example a mutation that results in a mutation in the LAZY4D motif (SEQ ID NO. 3, 4, 5, 6 or 73).

### Methods for Modulating Plant Traits/Producing Plants with Modulated Traits

In another aspect, the invention relates to a method for modulating plant traits comprising introducing a dominant gain of function mutation into a LAZY4 nucleic acid encoding for a protein having a LAZY4D motif (SEQ ID NO. 3, 4, 5, 6 or 73). In one embodiment, said trait is root growth. Thus, the invention relates to a method for conferring a steeper root angle to a plant comprising introducing a dominant gain of function mutation into a LAZY4 nucleic acid encoding for a protein having a LAZY4D motif (SEQ ID NO. 3, 4, 5, 6 or 73). In another embodiment, said trait is drought resistance or yield which are both increased according to the methods of the invention. Plant traits are modulated compared to a control plant as defined herein.

In another aspect, the invention relates to a method for producing a plant with modulated root growth, comprising introducing a dominant gain of function mutation into a LAZY4 nucleic acid encoding for a protein having a LAZY4D motif (SEQ ID NO. 3, 4, 5, 6 or 73).

In one embodiment, the methods comprise introducing a mutation into a LAZY4 nucleic acid sequence wherein said mutant LAZY4 nucleic acid sequence encodes a mutant LAZY4 protein comprising a mutation in the LAZY4D motif (SEQ ID NO. 3, 4, 5, 6 or 73). Thus, according to the various methods of the invention, the LAZY4 nucleic acid sequence is mutated compared to a wild type LAZY4 nucleic acid sequence, for example by targeted genome modification, thus encoding a mutant LAZY4 protein.

In one embodiment of the methods, one or more amino acid residue in the LAZY4D motif is substituted with another amino acid residue. In one embodiment, one or more of the following residues is substituted with another amino acid residue: C, P, S, S/C, L, E, V, D, R or R. In one embodiment, the residue mutated is the penultimate R. The one or more amino acid residue in the LAZY4D motif, for example the penultimate R, can be substituted with any natural amino acid residue.

In one embodiment, the (wild type) LAZY4 nucleic acid sequence comprises or consists of SEQ ID NO. 1 or a homolog, orthologue or functional variant thereof. This encodes a (wild type) LAZY4 protein comprising or consisting of SEQ ID NO. 2. As explained above, in one embodiment, the mutation resides in the conserved LAZY4D motif. Thus, according to the method of the invention, the plant may be a monocot or dicot plant. Such plants are exemplified above and include rice, maize, wheat and sorghum. Orthologues of SEQ ID NO. 1 that can be targeted/used according to the methods of the invention, for example by genome editing of the endogenous LAZY4 nucleic acid sequence are also listed above.

In one embodiment, the method comprises introducing the mutation using targeted genome modification (e.g. genome editing).

### Targeted Genome Modification Using Gene Editing

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customizable DNA binding proteins can be used: meganucleases derived from microbial mobile genetic elements, ZF nucleases based on eukaryotic transcription factors, rare-cutting endonucleases/sequence specific endonucleases (SSN), for example TALENs, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate their nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of Fokl to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat- variable di-residue (RVD). The RVD determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. The use of this technology in genome editing is well described in the art, for example in U.S. Pat. No. 8,440,431, U.S. Pat. No. 8,440, 432 and U.S. Pat. No. 8,450,471. Customized plasmids can be used with the Golden Gate cloning method to assemble multiple DNA fragments. The Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer).

The Type II CRISPR is one of the most well characterized systems and carries out targeted DNA double-strand breaks in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and a large monomeric DNA nuclease guided to a DNA target sequence adjacent to the PAM sequence motif by a complex of two noncoding RNAs: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a guide RNA (gRNA) also called single guide RNA (sgRNA) can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

Synthetic CRISPR systems typically consist of two components, the gRNA and a non-specific CRISPR-associated endonuclease and can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA. The genomic target can be any 20 nucleotide DNA sequence, provided that the target is present immediately upstream of a PAM sequence. The PAM sequence is of outstanding importance for target binding and the exact sequence is dependent upon the species of Cas9.

The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas9 and likewise CRISPR/Cpf1 and other CRISPR systems are highly specific when gRNAs are designed correctly, but especially specificity is still a major concern, particularly for clinical uses based on the CRISPR technology. The specificity of the CRISPR system is determined in large part by how specific the gRNA targeting sequence is for the genomic target compared to the rest of the genome.

The sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Thus, as used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

sgRNAs suitable for use in the methods of the invention are described below.

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be an RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also contemplated. The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeably herein to refer to a target sequence that is endogenous or native to the genome of a plant and is at the endogenous or native position of that target sequence in the genome of the plant.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

In one embodiment, the Cas endonuclease gene is a Cas9 endonuclease, such as but not limited to, Cas9 genes listed in WO2007/025097 incorporated herein by reference. In another embodiment, the Cas endonuclease gene is plant, maize or soybean optimized Cas9 endonuclease.

In one embodiment, the Cas endonuclease gene is a plant codon optimized Streptococcus pyogenes Cas9 gene that can recognize any genomic sequence of the form N(12-30)NGG can in principle be targeted.

In one embodiment, the Cas endonuclease is introduced directly into a cell by any method known in the art, for example, but not limited to transient introduction methods, transfection and/or topical application.

Cas9 expression plasmids for use in the methods of the invention can be constructed as described in the art and as described in the examples.

In one embodiment, targeted genome modification according to the various aspects of the invention comprises the use of a rare-cutting endonuclease, for example a TALEN, ZFN or CRISPR/Cas; e.g. CRISPR/Cas9. Rare-cutting endonucleases/ sequence specific endonucleases are naturally or engineered proteins having endonuclease activity and are target specific. These bind to nucleic acid target sequences which have a recognition sequence typically 12-40 bp in length. In one embodiment, the SSN is selected from a TALEN. In another embodiment, the SSN is selected from CRISPR/Cas9. This is described in more detail below.

In one embodiment, the step of introducing a mutation comprises contacting a population of plant cells with DNA binding protein targeted to an endogenous LAZY4 gene sequence, for example selected from the exemplary sequences listed herein. In one embodiment, the method comprises contacting a population of plant cells with one or more rare-cutting endonucleases; e.g. ZFN, TALEN, or CRISPR/Cas9, targeted to an endogenous LAZY4 gene sequence.

The method may further comprise the steps of selecting, from said population, a cell in which a LAZY4 gene sequence has been modified and regenerating said selected plant cell into a plant.

In an embodiment, the method comprises the use of CRISPR/Cas9. In this embodiment, the method therefore comprises introducing and co-expressing in a plant Cas9 and sgRNA targeted to a LAZY4 gene sequence and screening for induced targeted mutations in a LAZY4 nucleic gene. For example, the sgRNA targeted to the sequence in the gene that encodes the LAZY4D motif (SEQ ID NO. 3). The method may also comprise the further step of regenerating a plant and selecting or choosing a plant with an altered root phenotype, e.g. having a steeper root angle.

Cas9 and sgRNA may be comprised in a single or two expression vectors. The target sequence is a LAZY4 nucleic acid sequence as shown herein, in particular the part that encodes the LAZY4 motif.

In one embodiment, screening for CRISPR-induced targeted mutations in a LAZY4 gene comprises obtaining a DNA sample from a transformed plant and carrying out DNA amplification and optionally restriction enzyme digestion to detect a mutation in a LAZY4 gene.

In one embodiment, the restriction enzyme is mismatch-sensitive T7 endonuclease. T7E1 is an enzyme that is specific to heteroduplex DNA caused by genome editing.

PCR fragments amplified from the transformed plants are then assessed using a gel electrophoresis assay based assay. In a further step, the presence of the mutation may be confirmed by sequencing the LAZY4 gene. Genomic DNA (i.e. wt and mutant) can be prepared from each sample, and DNA fragments encompassing each target site are amplified by PCR. The PCR products are digested by restriction enzymes as the target locus includes a restriction enzyme site. The restriction enzyme site is destroyed by CRISPR- or TALEN-induced mutations by NHEJ or HR, thus the mutant amplicons are resistant to restriction enzyme digestion, and result in uncleaved bands. Alternatively, the PCR products are digested by T7E1 (cleaved DNA produced by T7E1 enzyme that is specific to heteroduplex DNA caused by genome editing) and visualized by agarose gel electrophoresis. In a further step, they are sequenced.

In one embodiment, the method uses the sgRNA (and template, synthetic single-strand DNA oligonucleotides (ssDNA oligos) or donor DNA) constructs defined in detail below to introduce a targeted SNP or mutation, in particular one of the substitutions described herein into a GRF gene and/or promoter. The introduction of a template DNA strand, following a sgRNA-mediated snip in the double-stranded DNA, can be used to produce a specific targeted mutation (i.e. a SNP) in the gene using homology directed repair. Synthetic single-strand DNA oligonucleotides (ssDNA oligos) or DNA plasmid donor templates can be used for precise genomic modification with the homology-directed repair (HDR) pathway. Homologous recombination is the exchange of DNA sequence information through the use of sequence homology. Homology-directed repair (HDR) is a process of homologous recombination where a DNA template is used to provide the homology necessary for precise repair of a double-strand break (DSB). CRISPR guide RNAs program the Cas9 nuclease to cut genomic DNA at a specific location. Once the double-strand break (DSB) occurs, the mammalian cell utilizes endogenous mechanisms to repair the DSB. In the presence of a donor DNA, either a ssDNA oligo or a plasmid donor, the DSB can be repaired precisely using HDR resulting in a desired genomic alteration (insertion, removal, or replacement).

Single-strand DNA donor oligos are delivered into a cell to insert or change short sequences (SNPs, amino acid substitutions, epitope tags, etc.) of DNA in the endogenous genomic target region.

A “donor sequence” is a nucleic acid sequence that contains all the necessary elements to introduce the specific substitution into a target sequence, preferably using homology-directed repair (HDR). In one embodiment, the donor sequence comprises a repair template sequence for introduction of at least one SNP. Preferably the repair template sequence is flanked by at least one, preferably a left and right arm, more preferably around 100 bp each that are identical to the target sequence. More preferably the arm or arms are further flanked by two gRNA target sequences that comprise PAM motifs so that the donor sequence can be released by Cas9/gRNAs. Donor DNA has been used to enhance homology directed genome editing (e.g. Richardson et al, Enhancing homology-directed genome editing by catalytically active and inactive CRISPR-Cas9 using asymmetric donor DNA, Nature Biotechnology, 2016 Mar.; 34(3): 339-44).

The methods above use plant transformation to introduce an expression vector comprising a sequence-specific nucleases into a plant to target a LAZY4 nucleic acid sequence. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle bombardment as described in the examples, transformation using viruses or pollen and microinjection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

The sequence-specific nucleases are is preferably introduced into a plant as part of an expression vector. The vector may contain one or more replication systems which allow it to replicate in host cells. Self-replicating vectors include plasmids, cosmids and virus vectors. Alternatively, the vector may be an integrating vector which allows the integration into the host cell's chromosome of the DNA sequence. The vector desirably also has unique restriction sites for the insertion of DNA sequences. If a vector does not have unique restriction sites it may be modified to introduce or eliminate restriction sites to make it more suitable for further manipulation. Vectors suitable for use in expressing the nucleic acids, are known to the skilled person and a non-limiting example is pYP010. The nucleic acid is inserted into the vector such that it is operably linked to a suitable plant active promoter. Suitable plant active promoters for use with the nucleic acids include, but are not limited to CaMV35S, wheat U6, or maize ubiquitin promoters.

### Conventional Mutagenesis Methods

As an alternative to the gene editing methods described above, more conventional mutagenesis methods can be used in the methods of the invention to introduce at least one mutation into a LAZY4 gene sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. In one embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 1 1, 2283-2290, December 1999).

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the LAZY4 nucleic acid sequence in a wild type plant and phenotypic analysis is also carried out. In another embodiment, mutagenesis is physical mutagenesis, such as application of ultraviolet radiation, X-rays, gamma rays, fast or thermal neutrons or protons. The targeted population can then be screened to identify a LAZY4 gain of function mutant. In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1 'EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy- 6-chloro-9 [3-(ethyl-2-chloroethyl)aminopropylamino]acridine dihydrochloride (ICR-170) or formaldehyde. Again, the targeted population can then be screened to identify a LAZY4 gene.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the LAZY4 target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the LAZY4 nucleic acid sequence may be utilized to amplify the LAZY4 nucleic acid sequence within the pooled DNA sample. Preferably, the primer is designed to amplify the regions of the LAZY4 gene where useful mutations are most likely to arise, specifically in the areas of the LAZY4 gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is a molecular technique that is similar to TILLING, except that its objective is to uncover natural variation in a given population as opposed to induced mutations.

Rapid high-throughput screening procedures thus allow the analysis of amplification products for identifying a dominant gain of function mutant as compared to a corresponding non-mutagenised wild type plant. Once a mutation is identified in a gene of interest, the seeds of the M2 plant carrying that mutation are grown into adult M3 plants and screened for the phenotypic characteristics associated with the target gene LAZY4. Gain of function mutants with altered root growth, i.e. a steeper root angle, compared to a control can thus be identified.

Plants obtained or obtainable by any of the methods described above method, such as plants which carry a gain of function mutation in the endogenous LAZY4 gene, are also within the scope of the invention.

### Transgenic Approaches

As discussed throughout, the inventors have surprisingly identified a new LAZY4 allele that acts as a dominant gain of function allele. Accordingly, overexpression of this allele in a wild-type or control plant will also increase grain yield and/or quality. Whilst the methods described above are directed to the manipulation of endogenous nucleic acids, e.g. LAZY4 targeted with a sequence specific endonuclease, convention transgenic approaches can alternatively be employed in the methods of the invention. Thus, the methods may comprise introducing a transgene into a plant of interest wherein said transgene comprises a LAZY4 nucleic acid with a dominant gain of function mutation. In one embodiment, the LAZY4 nucleic acid comprises a mutation that results in a mutation in the LAZY4D motif (e. g. SEQ ID NO. 3). The transgene may be operably linked to a suitable promoter, e.g. a promoter that overexpresses the gene, a tissue-specific promoter or a constitutive promoter. The promoter-LAZY4 transgene construct may be comprised in a suitable vector.

In yet another aspect of the invention there is provided a nucleic acid construct comprising a nucleic acid sequence encoding a polypeptide as defined in SEQ ID NO. 2 or a functional variant homolog/orthologue thereof, but which includes a dominant gain of function mutation, wherein said sequence is operably linked to a regulatory sequence. In one embodiment, said regulatory sequence is a promoter that overexpresses the gene, a tissue-specific promoter or a constitutive promoter. In one embodiment, the mutation in the nucleic acid sequence results in a protein that has a mutation in the LAZY4D motif.

A functional variant, homolog orthologue is as defined above. Promoters are also defined above.

The nucleic acid sequence is introduced into said plant through a process called transformation as described above. The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion). A suitable plant is defined above.

In another aspect, the invention relates to the use of a nucleic acid construct as described herein to modify root growth, in particular induce a steeper root angle, compared to a control plant.

### Constructs for Making Plants by Genome Editing

As explained above, in some embodiments, the methods of the invention use gene editing using sequence specific endonucleases that target a LAZY4 gene in a plant of interest. As also explained, Cas9 and gRNA may be comprised in a single or two expression vectors. The sgRNA targets the LAZY4 nucleic acid sequence. The target sequence in a LAZY4 nucleic acid sequence may be the LAZY4 motif as described herein.

Thus, in another aspect of the invention, there is provided a nucleic acid construct comprising a nucleic acid sequence encoding at least one DNA-binding domain that can bind to a LAZY4 gene. The LAZY4 gene comprises SEQ ID NO. 1 or a functional variant, homolog or orthologue thereof as explained herein.

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one LAZY4 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

In one embodiment, the nucleic acid sequence encodes at least one protospacer element.

In one embodiment, the construct further comprises a nucleic acid sequence encoding a CRISPR RNA (crRNA) sequence, wherein said crRNA sequence comprises the protospacer element sequence and additional nucleotides. In one embodiment, the construct further comprises a nucleic acid sequence encoding a transactivating RNA (tracrRNA).

In a further embodiment, the construct encodes at least one single-guide RNA (sgRNA), wherein said sgRNA comprises the tracrRNA sequence and the crRNA sequence, wherein the sgRNA comprises or consists of a sequence selected from any of SEQ IDs 45 to 60 listed herein, depending on the species targeted. PAM sequences are also shown in the in the section entitled sequences listing. The sgRNA can be used for manipulation of wheat and barley. In another aspect of the invention, there is provided a nucleic acid construct comprising a DNA donor nucleic acid wherein said DNA donor nucleic acid is operably linked to a regulatory sequence.

Cas9 and sgRNA may be combined or in separate expression vectors (or nucleic acid constructs, such terms are used interchangeably). Similarly, Cas9, sgRNA and the donor DNA sequence may be combined or in separate expression vectors. In other words, in one embodiment, an isolated plant cell is transfected with a single nucleic acid construct comprising both sgRNA and Cas9 or sgRNA, Cas9 and the donor DNA sequence as described in detail above. In an alternative embodiment, an isolated plant cell is transfected with two or three nucleic acid constructs, a first nucleic acid construct comprising at least one sgRNA as defined above, a second nucleic acid construct comprising Cas9 or a functional variant or homolog thereof and optionally a third nucleic acid construct comprising the donor DNA sequence as defined above. The second and/or third nucleic acid construct may be transfected before, after or concurrently with the first and/or second nucleic acid construct. The advantage of a separate, second construct comprising a Cas protein is that the nucleic acid construct encoding at least one sgRNA can be paired with any type of Cas protein, as described herein, and therefore is not limited to a single Cas function (as would be the case when both Cas and sgRNA are encoded on the same nucleic acid construct).

In one embodiment, a construct as described above is operably linked to a promoter, for example a constitutive promoter.

In another embodiment, the nucleic acid construct further comprises a nucleic acid sequence encoding a CRISPR enzyme. Preferably, the CRISPR enzyme is a Cas protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is Fokl.

In another aspect of the invention there is provided a single guide (sg) RNA molecule wherein said sgRNA comprises a crRNA sequence and a tracrRNA sequence.

In one embodiment, the sgRNA molecule may comprise at least one chemical modification, for example that enhances its stability and/or binding affinity to the target sequence or the crRNA sequence to the tracrRNA sequence. For example, the crRNA may comprise a phosphorothioate backbone modification, such as 2′-fluoro (2′-F), 2′-0-methyl (2′-0-Me) and S-constrained ethyl (cET) substitutions.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by an endoribonuclease cleavage site. The term ‘variant’ refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99% identity to any one of the above described sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissue-specific promoter. In one embodiment, suitable plant promoters are selected from, but not limited to, cestrum yellow leaf curling virus (CmYLCV) promoter or switchgrass ubiquitin 1 promoter (PvUbil) wheat U6 RNA polymerase III (TaU6) CaMV35S, wheat U6 or maize ubiquitin (e.g. Ubi 1) promoters. Alternatively, expression can be specifically directed to particular tissues of wheat seeds through gene expression-regulating sequences.

The nucleic acid construct of the present invention may also further comprise a nucleic acid sequence that encodes a CRISPR enzyme. In a specific embodiment Cas9 is codon-optimised Cas9. In another embodiment, the CRISPR enzyme is a protein from the family of Class 2 candidate proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides or Streptococcus thermophiles.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active.

In a further embodiment, the Cas9 protein has been modified to improve activity. Suitable homologs or orthologs can be identified by sequence comparisons and identifications of conserved domains. The function of the homolog or ortholog can be identified as described herein and a skilled person would thus be able to confirm the function when expressed in a plant. In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas protein may comprise a mutation in both nuclease domains, HNH and RuvC-like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains.

In a further embodiment, a Cas protein, such as Cas9 may be further fused with a repression effector, such as a histone-modifying/DNA methylation enzyme or a Cytidine deaminase to effect site-directed mutagenesis. In the latter, the cytidine deaminase enzyme does not induce dsDNA breaks, but mediates the conversion of cytidine to uridine, thereby effecting a C to T (or G to A) substitution. These approaches may be particularly valuable to target glutamine and proline residues in gliadins, to break the toxic epitopes while conserving gliadin functionality.

In a further embodiment, the nucleic acid construct comprises an endoribonuclease. Preferably the endoribonuclease is Csy4 (also known as Cas6f) and more preferably a codon optimised csy4. In one embodiment, where the nucleic acid construct comprises a Cas protein, the nucleic acid construct may comprise sequences for the expression of an endoribonuclease, such as Csy4 expressed as a 5′ terminal P2A fusion (used as a self-cleaving peptide) to a Cas protein, such as Cas9.

In one embodiment, the Cas protein, the endoribonuclease and/or the endoribonuclease-Cas fusion sequence may be operably linked to a suitable plant promoter. Suitable plant promoters are already described above, but in one embodiment, may be the Zea mays Ubiquitin 1 promoter.

Suitable methods for producing the CRISPR nucleic acids and vectors system are known, and for example are published in Molecular Plant (Ma et al., 2015, Molecular Plant, 2015 Aug.;8(8):1274-8), which is incorporated herein by reference.

In a further aspect of the invention, there is provided an isolated plant cell transfected with at least one nucleic acid construct as described herein. In one embodiment, the isolated plant cell is transfected with at least one nucleic acid construct as described herein and a second nucleic acid construct, wherein said second nucleic acid construct comprises a nucleic acid sequence encoding a Cas protein, preferably a Cas9 protein or a functional variant thereof. Preferably, the second nucleic acid construct is transfected before, after or concurrently with the first nucleic acid construct described herein.

In an alternative aspect of the invention, the nucleic acid construct comprises at least one nucleic acid sequence that encodes a TAL effector.

In a further aspect of the invention there is provided a genetically modified plant, wherein said plant comprises the transfected cell as described herein. Preferably, the nucleic acid encoding the sgRNA and/or the nucleic acid encoding a Cas protein is integrated in a stable form.

Also included in the scope of the invention, is the use of the nucleic acid constructs (CRISPR constructs) described above or the sgRNA molecules in any of the above described methods. For example, there is provided the use of the above CRISPR constructs or sgRNA molecules to modulate LAZY4 activity as described herein. In particular, as described herein, the CRISPR constructs may be used to create dominant gain of function alleles.

In a yet further aspect of the invention there is provided a method of altering root growth in a plant, the method comprising introducing and expressing in a plant a nucleic acid construct as described herein. In another aspect of the invention there is provided a method for obtaining the genetically modified plant as described herein, the method comprising:
a. selecting a part of the plant;
b. transfecting at least one cell of the part of the plant of paragraph (a) with the nucleic acid construct as described above;
c. regenerating at least one plant derived from the transfected cell or cells; selecting one or more plants obtained according to paragraph (c) that show altered root growth.

### Isolated Mutant Nucleic Acids/Protein

The invention also relates to an isolated mutant LAZY4 nucleic acid sequence encoding a mutant LAZY4 protein comprising a dominant gain of function mutation.

In one embodiment, the isolated mutant LAZY4 nucleic acid sequence encodes a mutant LAZY4 protein comprising a modification in the LAZY4D motif (SEQ ID NO. 3, 4, 5, 6 or 73).

In one embodiment, the mutant LAZY4 protein comprises a substitution of one or more amino acid residue in the LAZY4D motif with another amino acid residue. Thus, any residue in SEQ ID NO. 3, 4, 5, 6 or 73 may be substituted, for example with A or G. In one embodiment, one or more amino acid residue in the LAZY4D motif is substituted with another amino acid residue. In one embodiment, one or more of the following residues is substituted with another amino acid residue: L, P, D, R, F, N, C, S, E, V, In one embodiment, one or more of the following residues is substituted with another amino acid residue: C, P, S, L, E, V, D, R or R. In one embodiment, the residue mutated is the penultimate R. The one or more amino acid residue in the LAZY4D motif, for example the penultimate R, can be substituted with any natural amino acid residue.

In one embodiment, the isolated mutant LAZY4 nucleic acid sequence is mutated compared to a wild type sequence, e.g. SEQ ID NO. 1 or a homolog, orthologue or functional variant thereof as defined elsewhere herein. Thus, the LAZY4 nucleic acid may be that of a dicot or monocot plant. Examples of wild type LAZY4 nucleic acid sequences are listed elsewhere herein and include SEQ ID NOs. 8, 10, 12, 14, 16, 18, 20, 22, 24, 26, 28, 30, 32, 34, 36, 38, 40, 42, 44, 62, 64, 66, 68, 70, 72. Examples of wild type LAZY4 amino acid sequences are listed elsewhere herein and include SEQ ID NOs. 7, 9, 11, 13, 15, 17, 19, 21, 23, 25, 27, 29, 31, 33, 35, 37, 39, 41, 43, 61, 63, 65, 67, 69, 71.

The invention also relates to a vector comprising an isolated nucleic acid described above.

The invention also relates to a host cell comprising an isolated nucleic acid or vector as described above. The host cell may be a plant cell or a microbial cell. The host cell may be a bacterial cell, such as Agrobacterium tumefaciens, or an isolated plant cell. The invention also relates to a culture medium or kit comprising a culture medium and an isolated host cell as described below.

### Methods and Kits for Identifying a Plant with Altered Root Growth

The invention also relates to a method for identifying a plant with altered root growth compared to a control plant comprising detecting in a population of plants or plant germplasm one or more polymorphisms in a LAZY4 nucleic acid sequence (SEQ ID NO. 1) wherein the control plant is homozygous for a LAZY4 nucleic acid that encodes a protein having a wild type LAZY4D motif (SEQ ID NO. 3). For example, the polymorphism is in the LAZY4D motif. In one embodiment, the polymorphism is an insertion, deletion and/or substitution.

In one embodiment, the method further comprises introgressing the chromosomal region comprising at least one polymorphism in the LAZY4 gene into a second plant or plant germplasm to produce an introgressed plant or plant germplasm.

The invention also relates to a detection kit for determining the presence or absence of a polymorphism in the LAZY4D motif (SEQ ID NO. 3, 4, 5, 6 or 73) encoded by a LAZY4 nucleic acid sequence in a plant.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a mutant plant cell, tissue, organ or whole plant that has been produced by any of the aforementioned methods, the only requirement being that progeny exhibit the same genotypic and/or phenotypic characteristic(s) as those produced by the parent in the methods according to the invention. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1. “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is further described in the following non-limiting examples.

### EXAMPLES

### Example 1: Identification of a Single Nucleotide Mutation in the LAZY4 Gene of Arabidopsis that Results in More Vertical Lateral Root Growth

Approximately 20,000 seeds of Arabidopsis wt Col-0 were subject to random mutagenesis using 25mM Ethylmethane Sulphonate (EMS) overnight. The EMS was neutralised and the mutagenized seeds were sown out to grow to maturity, the plants resulting from the mutagenized seeds are known as the M1 generation. Seed from the M1 plants was collected, this seed was sterilised and grown on vertically placed plates of ATS (Arabidopsis Thaliana Salts) agar at 20° C. constant 16 hour days for 12 days. The plates were then photographed and visually inspected for root angle mutants, the LAZY4D (at this stage only known by a number) mutant was selected at this stage because of its strikingly vertical lateral roots. This plant (M2) was then placed into soil and allowed to grow to maturity and produce seed.

In order to genotype the mutant, M3 plants of LAZY4D were back-crossed with wt Col-0. The resultant F1 progeny all displayed the more vertical lateral root phenotype indicating that the mutation was dominant. The F2 plants displayed a 3:1 segregation ratio of more vertical root phenotype:no phenotype (this ratio indicates that the phenotype was caused by a mutation in a single gene), a small sample of leaf tissue was taken from each plant and frozen using liquid Nz. Each plant displaying the phenotype was grown to produce seed, the F3 offspring were then phenotyped, those which displayed segregation were the product of a heterozygous F2 parent. Two pools containing tissue from 50 F2 plants that were homozygous for either the phenotype or no phenotype were created and genomic DNA was extracted from these. The DNA from both the Phenotype and No Phenotype pools was whole genome sequenced and the sequence assembled against the TAIR 10 reference sequence. Single nucleotide polymorphisms were called for both pools, those that appeared in only the Phenotype pool were listed as potential causal mutations.

Of these potential mutations it was decided that the most likely causal mutation would be the one in LAZY4 (see SEQ ID NO. 1 and 2) as the gene was already known to have some control over lateral root growth angle. The single nucleotide change in LAZY4 resulted in a R145K amino acid change. In order to prove this was the causal mutation LAZY4 was cloned from both wt Col-0 and the original mutant and put under the control of the native promoter using gateway cloning. The construct containing LAZY4 cloned from wt Col-0 was then subject to site directed mutagenesis to replicate the base change from the mutant (R145K) and to introduce other amino acid changes (R145A and R145E). These constructs (pLAZY4:LAZY4, pLAZY4:LAZY4 R145LAZY4D, pLAZY4:LAZY4 R145K, pLAZY4:LAZY4 R145A and pLAZY4:LAZY4 R145E) were transformed into the knockout mutant atlazy4 using Agrobacterium mediated transformation. The resultant T1 progeny were phenotyped, the pLAZY4:LAZY4 T1 displayed a wt phenotype confirming that the construct functioned. All the other constructs that contained a mutation in R145 of LAZY4 displayed the more vertical lateral root phenotype confirming that the change at R145 of LAZY4 was the cause of the more vertical lateral root phenotype and that it was the loss of the R at that position rather than a gain of an alternative amino acid that resulted in the change.

This is shown in FIGS. 1 and 2.

### Example 2: Introducing the lazy4D Mutation Into the LAZY4 Paralogue

LAZY2 was cloned from wt Col-0 and put under the control of its native promoter using gateway cloning. Site directed mutagenesis was used to introduce an R143A change into the LAZY2 protein sequence. The pLAZY2:LAZY2 R143A construct was transformed into wt Col-0 using Agrobacterium mediated transformation. The resultant T1 progeny were grown and phenotyped as for the original LAZY4D mutant, all displayed more vertical lateral root growth. The construct was also transformed into the lazy2 knockout mutant, the T1 generation of this transformation also displayed more vertical lateral root growth.

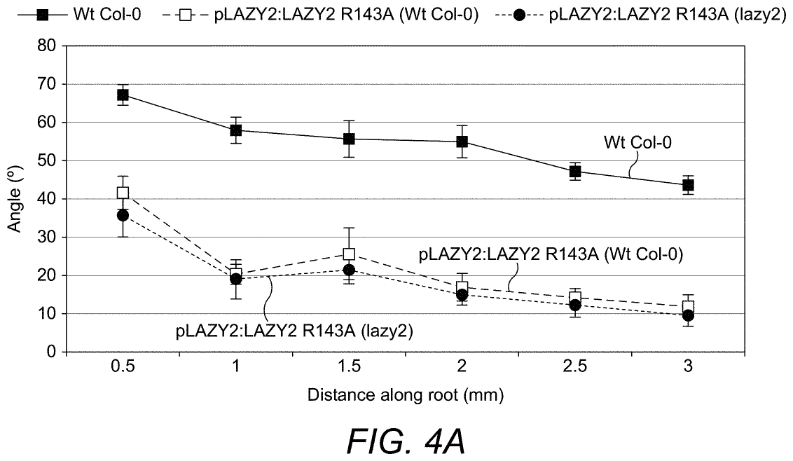

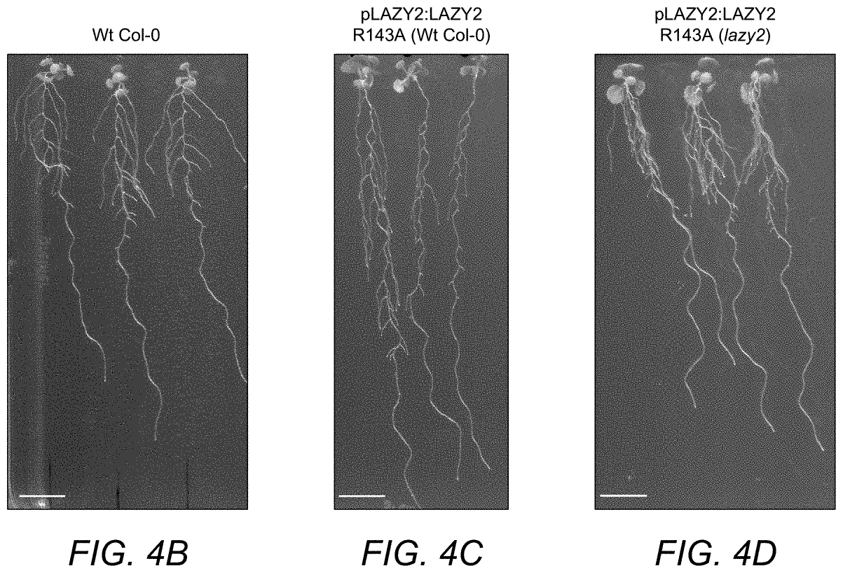

This is shown in FIG. 4.

### Example 3: Mutation of Other Residues in the 4D Motif

LAZY4 was cloned from wt Col-0 and put under the control of its native promoter using gateway cloning. Site directed mutagenesis was used to introduce a C137A, P138A, V143A, D144A, R146A, S139A, L129A, P130A or R133A change into the LAZY4 protein sequence. The pLAZY4:LAZY4 C137A, pLAZY4:LAZY4 P138A, pLAZY4:LAZY4 V143A, pLAZY4:LAZY4 D144A, pLAZY4:LAZY4 R146A, pLAZY4:LAZY4 S139A, pLAZY4:LAZY4 L129A, pLAZY4:LAZY4 P130A, pLAZY4:LAZY4 R133A constructs were generated and are transformed into the knockout mutant atlazy4 and wt Col-0 using Agrobacterium mediated transformation. The resultant T1 progeny are grown and phenotyped as for the original LAZY4D mutant.

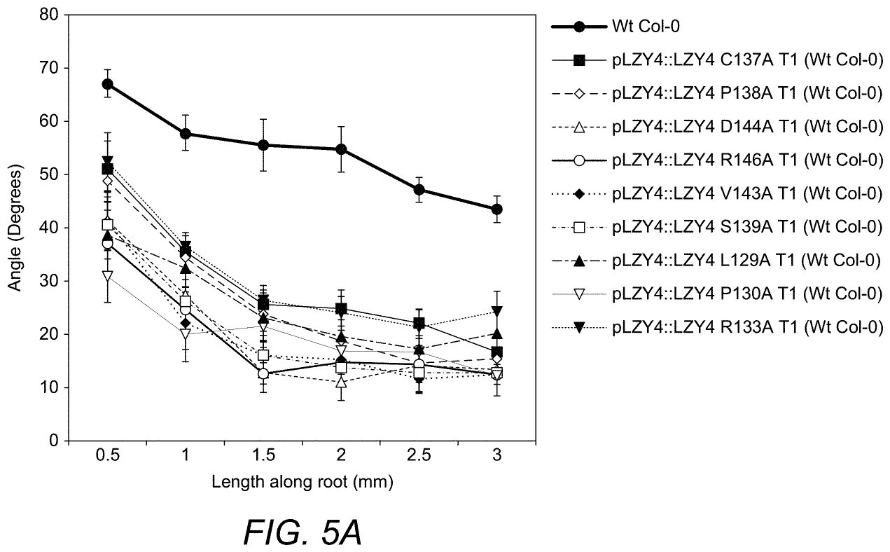

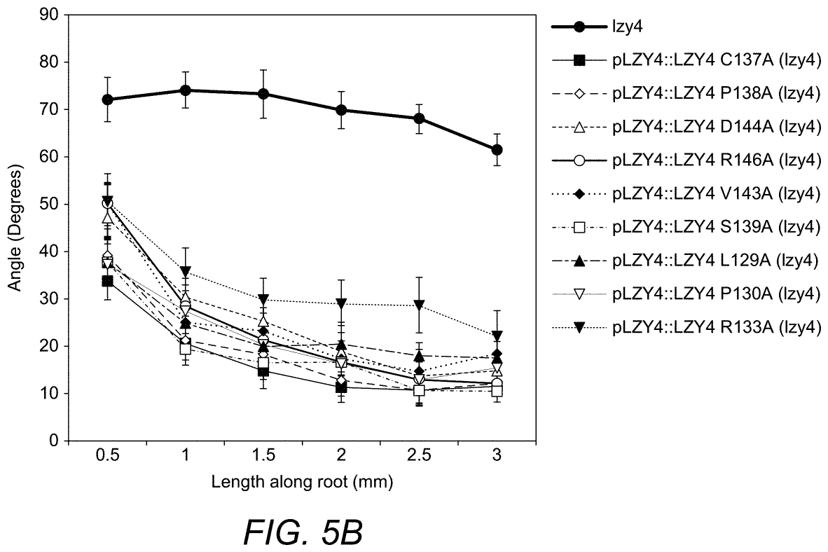

Site directed mutagenesis of the above mentioned residues in the AtLAZY4 motif also resulted in significantly more vertical lateral roots than wt, these mutations are also dominant as when transformed into wt Col-0 the significantly more vertical lateral root phenotype is present in the T1 generation, this is shown in FIG. 5.

### Example 4: Exemplification the lazy4D Technology Using Gene Editing

The technology is exemplified in other plants, e.g. wheat using two approaches.

The first approach is a conventional transgenic approach. A wheat homolog of LAZY4 and its promoter is cloned and the LAZY4D mutation is introduced using site directed mutagenesis. This construct containing the native promoter and mutant LAZY4 is then be transformed into wheat and the root phenotype is analysed, using standard techniques, such as Agrobacterium mediated transformation.

### Genome Editing

The second approach involves using a targeted base editing system based upon CRISPR-Cas9, for example fused to the APOBEC1 cytosine deaminase. The Cas9 along with the guide RNA directs the deaminase to the target site allowing the deaminase to convert cytosine to uracil, a uracil DNA glycosylase inhibitor inhibits the retaining of the uracil whilst a nickase nicks the opposite strand encouraging the cell's DNA repair machinery to use the uracil as the template for repair.

The use of RNA-guided Cas9 for genome editing in plants has been a major breakthrough, both as a valuable research tool and as a technology for development of improved crops. The range of genome editing tools continues to grow, and tools that allow precise base editing are offering exciting new opportunities.

The first base editing tools were described in mammalian cells then applied to plants. These allowed the substitution of cytosine (C) to thymine (T) or Guanine (G) to Adenine (A). This capability is provided by the APOBEC1 editing enzyme. Base editing works by fusing the editor to an inactive Cas9 (dCas9) or to a Cas9 nickase (nCas9). This is then guided to the target site by single guide RNA (sgRNA) where it binds. The final outcome is the base conversion C to T or G to A.

This technology has been used successfully in a range of cereal crops including wheat. A second editor allows an A to T or G to C change although this has been shown to be less efficient in plants. One limitation of this technology is the requirement for the protospacer adjacent motif (PAM); NGG is required with Cas9. However, there are now modified Cas9 nucleases that have more relaxed PAM requirements making it easier to design base-editing strategies.

The following protocol can be used although it is noted that alternatives to the CRISPR Cas9 system are now widely available, for example systems that use a different endonuclease, such as MAD7.

1. Design of sgRNA and CRISPR-Cas9 System

CRISPR-Cas systems for use in genome editing in crops have been disclosed elsewhere (e.g. Ma et al., 2015, Molecular Plant, 2015 Aug.;8(8):1274-8, Jaganathan et al., Front. Plant Sci., 17 2018).

For genome engineering applications, the type II CRISPR/Cas system minimally requires the Cas9 protein and a duplexed crRNA/tracrRNA molecule or a synthetically fused crRNA and tracrRNA (guide RNA) molecule for DNA target site recognition and cleavage (Gasiunas et al. (2012) Proc. Natl. Acad. Sci. USA). Thus, the methods employed to target LAZY4 and introduce a mutation in the LAZY4 motif can use a guideRNA/Cas endonuclease system that is based on the type II CRISPR/Cas system and consists of a Cas endonuclease and a guide RNA (or duplexed crRNA and tracrRNA) that together can form a complex that recognizes a genomic target site in a plant and introduces a double-strand-break into said target site.

The sgRNA for introducing an amino acid substitution into the target locus is designed based on the LAZY4 target sequence in the plant species of interest, e.g. rice, wheat, maize etc. Exemplary LAZY4 gene sequences are provided herein.

Target genomic sequences, i.e. LAZY4 gene sequences from plant species of interest, are analyzed using available tools to generate candidate sgRNA sequences. The sgRNA sequences can be generated by web-tools including, but not limited to, the web sites:

http://cbi.hzau.edu.cn/crispr or http://www.rgenome.net/be-designer/

Both tools are available online.

Exemplary sgRNA sequences are shown below (SEQ ID Nos. 45-60).

A CRISPR-Cas9 system can be used that utilises a suitable promoter and other components to optimise expression in the target plant species, e.g. the maize Ubi promoter, to drive the optimized coding sequence of Cas9 protein in maize or the GhU6 promoter to drive expression in cotton, AtU6 (for Arabidopsis); TaU6 (for wheat); OsU6 or OsU3 (for rice).

Other elements include CAMV35S 3′-UTR as this improves expression of the Cas9 protein.

One sgRNA can be used to make the genome editing construct. The single sgRNA can guide the Cas9 enzyme to the target region and generate the double strand break at the target DNA sequence, non-homologous end-joining (NHEJ) repairing mechanism and homology directed repair (HOR) will be triggered, and it often induces random insertion, deletion and substitution at the target site.

Alternatively, two sgRNAs can be used to make the genome editing construct. This construct can lead to fragment deletion, point mutation (small insertion, deletion and substitution).

Another component that can be included to form a functional guide RNA/Cas endonuclease system for genome engineering applications is a duplex of the crRNA and tracrRNA molecules or a synthetic fusing of the crRNA and tracrRNA molecules, a guide RNA. The guide RNA or crRNA molecule may also contain a region complementary to one strand of the double strand DNA target that is approximately 12-30 nucleotides in length and upstream of a PAM sequence.

Expression of both the Cas endonuclease gene and the guide RNA then allows for the formation of the guide RNA/Cas complex.

There are several commercially available vectors for expressing Cas9 or Cas9 variants and gRNAs in plant.

### 2. Plant Transformation

Plants are transformed with the vector using standard techniques, for example biolistic transformation (e.g. in wheat or maize), protoplast transfection, electroporation of protoplasts or Agrobacterium mediated transformation (e.g. in rice).

### 3. Plant Selection

Plants are selected based on a phenotypic analysis and by sequences the target locus to confirm the mutation in the target sequence. Plants are for example grown on soil in controlled environment chambers. Genomic DNA from individual plants is extracted using standard techniques. PCR/RE digestion screen assays and sequencing can be used to identify the mutation present. Selectable marker genes that confer antibiotic or herbicide resistance can optionally be used, as well as visual markers.

Phenotypic analysis is carried out by assessing the root phenotype compared to a control plant that does not have the mutation, similar to the experiments shown in example 1.

An exemplary sgRNA for use in a method using targeted genome modification was designed for transformation in wheat and barley. The sgRNA nucleic acid sequence is: 5′-TCGACCGGCGGCTCTCGCTC-3 (SEQ ID. 45). This is being used for gene editing of the LAZY4 target sequence in wheat and in barley.

sgRNA sequences having SEQ ID NOs 46 to 60 can be used in targeting other species, such as Zea mays, tomato, rice, tobacco, oilseed rape and others. These sequences and their target species are shown below.

Sequences
AtLAZY4
SEQ ID NO: 1
MKFFGWMQNKLHGKQEITHRPSISSASSHHPREEFNDWPHGLLAIGTFGNKKQTPQTLDQEVI
QEETVSNLHVEGRQAQDTDQELSSSDDLEEDFTPEEVGKLQKELTKLLTRRSKKRKSDVNREL
ANLPLDRFLNCPSSLEVDRRISNALCDEKEEDIERTISVILGRCKAISTESKNKTKKNKRDLS
KTSVSHLLKKMFVCTEGFSPVPRPILRDTFQETRMEKLLRMMLHKKVNTQASSKQTSTKKYLQ
DKQQLSLKNEEEEGRSSNDGGKWWKTDSDFIVLEI
AtLAZY4
SEQ ID NO: 2
ATGAAGTTTTTCGGGTGGATGCAGAACAAGCTACATGGGAAACAAGAGATTACTCATAGACCA
AGCATATCCTCTGCTTCTTCTCATCATCCGAGAGAGGAGTTTAACGATTGGCCTCACGGATTA
CTCGCGATTGGTACATTCGGTAACAAAAAGCAGACACCACAAACACTTGATCAAGAAGTGATT
CAAGAAGAGACAGTGTCTAACTTACACGTGGAAGGTCGTCAAGCACAAGATACAGATCAAGAG
CTTTCTTCCTCCGATGATCTAGAAGAAGATTTCACTCCCGAAGAAGTTGGGAAACTACAGAAG
GAGCTGACGAAACTCTTGACGAGAAGGAGTAAGAAAAGGAAGTCTGATGTGAATCGAGAATTA
GCGAATCTTCCTTTGGATAGATTCTTGAATTGTCCTTCGAGTCTTGAGGTCGATAGAAGAATC
AGTAACGCGCTTTGTGATGAGAAGGAGGAAGACATTGAGCGTACAATCAGTGTTATCCTAGGG
AGATGCAAAGCTATTTCTACAGAGAGCAAGAACAAGACGAAGAAGAATAAAAGAGATTTGAGC
AAAACCTCTGTTTCTCATCTTCTCAAGAAGATGTTTGTCTGTACAGAAGGTTTTTCTCCCGTT
CCTCGCCCTATCTTGAGAGACACGTTTCAAGAAACAAGAATGGAGAAGTTGCTGAGAATGATG
CTACACAAGAAAGTTAACACTCAAGCTTCATCAAAGCAAACATCGACAAAAAAATACTTGCAA
GACAAGCAACAGCTCTCGTTGAAGAACGAGGAAGAAGAAGGACGAAGCAGTAACGATGGGGGG
AAATGGGTCAAAACAGATTCTGATTTCATTGTTCTTGAGATCTGA
LAZY4D motif
SEQ ID NO: 3
CPSXLEVDRR
X is any naturally occurring amino acid
LAZY4D motif
SEQ ID NO: 4
CPSSLEVDRR
LAZY4D motif
SEQ ID NO: 5
LANLPLDRFLNCPSSLEVDRRISNAL
SEQ ID NO: 6
X₁X₁X₁X₂LPLDRFLNCPSXLEVDRRX₁X₁X₁X₁X₁
>AtLAZY2
SEQ ID NO: 7
MKFFGWMQNKLNGDHNRTSTSSASSHHVKQEPREEFSDWPHALLAIGTFGTTSNSVSENESKN
VHEEIEAEKKCTAQSEQEEEPSSSVNLEDFTPEEVGKLQKELMKLLSRTKKRKSDVNRELMKN
LPLDRFLNCPSSLEVDRRISNALSAVVDSSEENKEEDMERTINVILGRCKEISIESKNNKKKR
DISKNSVSYLFKKIFVCADGISTAPSPSLRDTLQESRMEKLLKMMLHKKINAQASSKPTSLTT
KRYLQDKKQLSLKSEEEETSERRSSSDGYKWVKTDSDFIVLEI
AtLAZY2
SEQ ID NO: 8
ATGAAGTTCTTCGGGTGGATGCAGAACAAGCTTAATGGGGATCATAACAGAACAAGCACTTCC
TCTGCTTCTTCTCATCATGTGAAGCAAGAACCAAGAGAGGAGTTTAGCGACTGGCCTCACGCG
CTGCTTGCTATTGGAACATTCGGTACAACAAGCAATAGTGTGAGCGAAAACGAGAGCAAGAAT
GTTCATGAAGAGATTGAAGCGGAGAAGAAGTGTACGGCACAATCCGAGCAAGAAGAAGAGCCT
TCTTCCTCTGTCAATCTTGAGGATTTCACTCCTGAAGAGGTTGGAAAGTTGCAGAAAGAGTTG
ATGAAGCTCTTGTCAAGAACTAAGAAAAGGAAGTCTGATGTGAATAGAGAGCTCATGAAAAAT
CTTCCTTTAGATAGATTCTTGAACTGTCCATCGAGTTTAGAGGTGGATAGGCGAATCAGCAAT
GCGCTTAGCGCTGTTGTGGATTCGTCAGAGGAGAATAAGGAGGAAGATATGGAGCGAACGATT
AACGTTATTCTAGGTAGATGCAAAGAGATATCAATAGAGAGTAAGAATAACAAGAAGAAGAGA
GACATAAGCAAGAACTCTGTCTCATATCTTTTCAAGAAGATTTTTGTCTGCGCAGATGGGATT
TCTACAGCCCCAAGCCCTAGCTTGAGAGACACGCTTCAAGAATCAAGAATGGAGAAGTTGTTG
AAGATGATGCTCCATAAGAAGATTAATGCTCAAGCCTCCTCGAAACCAACATCATTGACAACA
AAGAGATACTTGCAAGACAAGAAACAGCTCTCACTGAAGAGTGAGGAAGAAGAAACTAGCGAA
AGAAGAAGTAGTAGCGATGGATATAAATGGGTCAAAACAGATTCTGATTTCATAGTTCTCGAG
ATATGA
Maize
ZmLAZY4
SEQ ID NO: 9
MQDRFNGKHDKRRPEAINSGSARESCRQDDRAREGKSRNDGGDWPAPQHGLLSIGTLGDDDPP
PPRASSQADDVLDFTIEEVKKLQDALNKLLRRAKSKSSSSSSSSRGSGASATDEDRRASHSQL
PLDRFLNCPSSLEVDRRVSLIRHDGGGESGEFSPDTQIILSKARDLLVHSNGTAIRKKSFKFL
LKKMFVCHGGFAPAPSLKDPVESRMEKLFRTMLQKKMNARPSNAAVSSRKYYLDDKPSGRMMT
RDGRRRHDGEDDDEKGSDRIKWDKTDTDCKNIFIRC
ZmLAZY4
SEQ ID NO: 10
ATGCAGGATCGCTTCAACGGTAAACACGATAAGAGGCGACCCGAGGCCATTAACTCGGGATCA
GCTCGCGAAAGCTGCCGCCAAGACGACCGCGCGCGCGAGGGCAAGAGCCGCAACGACGGCGGC
GACTGGCCGGCGCCACAGCACGGCCTCCTGTCGATCGGGACGCTGGGAGACGACGACCCGCCG
CCGCCGCGCGCGTCGTCGCAGGCCGACGACGTGCTGGACTTCACCATCGAGGAGGTGAAAAAG
CTCCAGGACGCGCTGAACAAGCTGCTCCGGCGCGCCAAGTCCAAGTCCAGCTCCAGCTCCAGC
TCCTCCCGCGGGTCGGGCGCCAGCGCCACCGACGAGGACCGCCGCGCCAGCCACAGCCAGCTG
CCGCTCGACAGGTTCCTCAACTGCCCCTCCAGCCTCGAGGTCGACCGGAGGGTCTCGCTGATC
AGGCACGACGGTGGTGGCGAGAGCGGCGAGTTCTCGCCGGACACGCAGATCATACTCAGCAAG
GCCAGGGATCTCCTCGTCCACAGCAACGGCACCGCCATCAGGAAGAAGTCGTTCAAGTTCCTC
CTGAAGAAGATGTTCGTCTGCCATGGCGGCTTCGCCCCCGCGCCGAGCTTGAAGGATCCAGTT
GAATCGAGAATGGAGAAGTTGTTCAGAACGATGCTTCAGAAGAAGATGAATGCTCGCCCGAGC
AACGCTGCAGTGTCATCCAGGAAGTACTACCTCGACGACAAGCCGAGCGGGAGGATGATGACA
CGGGATGGTCGTCGTCGTCACGATGGAGAGGACGATGACGAGAAGGGCTCTGACAGAATCAAG
TGGGATAAAACTGATACTGACTGTAAGAACATATTTATACGCTGCTAG
Soybean
Glycine max GmLAZY4.1
SEQ ID NO: 11
MKFLSWMQNKLGGKQDNRKPNTHTTNTTTYLAKQEPREEFSDWPHGLLAIGTFGNKSEIKEDL
DDQNTQEDPSSSEEIADFTPEEIGNLQKELTKLLRRKPNVEKEISELPLDRFLNCPSSLEVDR
RISNALCSESEDKEEDIEKTLSVIIDKCKDICADKRKKAIGKKSISFLLKKIFVCRSGFAPTP
SLRDTLQESRMEKLLRTMLHKKIYTQNSSRSPLVKKGIEDKKMTRKRNEDESDERNGDGCKWV
KTDSEYIVLEI
GmLAZY4.1
SEQ ID NO: 12
ATGCACTCTAAGCTCATTCATCCCCCCCTATCTTTTAGCCTTAGTCCTTCCACAATGAAGTTC
CTCAGCTGGATGCAAAATAAACTTGGTGGAAAACAAGACAACAGAAAACCAAATACACATACT
ACTAATACTACTACATATCTTGCAAAACAAGAGCCTAGAGAAGAATTCAGCGATTGGCCTCAT
GGTTTACTAGCAATTGGAACATTTGGAAATAAGAGTGAAATCAAAGAAGACTTAGACGACCAA
AATACACAAGAGGATCCATCTTCATCAGAGGAAATAGCAGACTTCACTCCTGAAGAAATTGGG
AATCTACAGAAGGAGTTAACTAAACTCCTGAGACGAAAACCCAATGTGGAAAAGGAAATTTCT
GAGCTCCCTCTGGACAGATTTCTTAACTGCCCTTCAAGCTTGGAGGTTGATAGGAGAATCAGT
AATGCACTATGCAGTGAATCAGAAGATAAGGAAGAAGATATTGAGAAGACACTGAGTGTGATA
ATTGATAAATGCAAAGACATTTGTGCAGATAAAAGAAAGAAAGCAATTGGGAAGAAATCCATT
TCTTTCCTTCTGAAGAAGATATTTGTTTGTAGAAGTGGATTTGCTCCAACACCTAGCCTAAGA
GATACCCTTCAAGAGTCAAGAATGGAGAAGCTTTTGAGGACAATGCTTCACAAGAAAATTTAC
ACCCAAAACTCTTCTCGGTCACCGTTGGTGAAGAAGGGCATAGAGGATAAGAAGATGACAAGG
AAGAGGAATGAGGATGAATCAGATGAGAGAAATGGTGATGGCTGTAAATGGGTCAAGACTGAT
TCTGAATATATTGTTCTAGAGATATAA
Glycine max GmLAZY4.2
SEQ ID NO: 13
MHSKLVHPPLSFSLSPSTMKFLSWMQNKLGGKQDNRKPNAHTTTTTTTTTYHPKQEPREEFSD
WPHGLLAIGTFGNKTAIKEDLDDQNTQEDPSSSEEIADFTPEEIGNLQKELTKLLRRKPNVEK
EISELPLDRFLNCPSSLEVDRRISNALCSESEDKEEDIEKTLSVIIDKCKDICADKRKKAMGK
KSISFLLKKIFLCRSGFAPTPSLRDTLQESRMEKVLRTMLHKKICTQNSSRSPLVKKCIEDKK
MTRKKNEDESDERNGDGCKWWKTDSEYIVLEI
GmLAZY4.2
SEQ ID NO: 14
ATGCACTCTAAGCTCGTTCATCCCCCCCTATCTTTTAGCCTTAGTCCTTCCACAATGAAGTTC
CTCAGCTGGATGCAAAATAAACTTGGTGGAAAACAAGACAACAGAAAACCAAATGCACATACT
ACAACAACTACTACTACTACTACATATCATCCAAAACAAGAGCCTAGGGAAGAATTCAGCGAT
TGGCCTCATGGTTTACTAGCGATTGGAACATTTGGAAACAAGACTGCAATCAAAGAAGACTTG
GATGACCAAAATACACAAGAGGATCCATCTTCTTCAGAGGAAATAGCAGACTTCACTCCTGAA
GAAATTGGGAATCTACAGAAGGAGTTAACTAAACTTCTGAGACGAAAACCCAATGTGGAAAAG
GAGATTTCTGAGCTTCCTCTGGACAGATTTCTTAACTGTCCTTCAAGCTTGGAGGTTGATAGG
AGAATCAGTAATGCACTATGCAGTGAATCAGAAGATAAGGAAGAAGATATTGAGAAAACACTA
AGTGTAATAATTGATAAATGCAAAGACATTTGTGCAGATAAAAGAAAGAAAGCAATGGGGAAG
AAATCTATTTCTTTCCTTCTGAAGAAGATCTTTCTTTGTAGAAGTGGATTTGCTCCAACACCA
AGCCTTAGAGATACCCTTCAAGAGTCAAGAATGGAGAAGGTTTTGAGGACAATGCTCCACAAG
AAAATTTGCACCCAAAATTCTTCTCGGTCACCGTTGGTGAAGAAGTGCATAGAGGACAAAAAG
ATGACAAGGAAGAAAAATGAGGATGAATCAGATGAGAGAAATGGTGATGGCTGTAAATGGGTC
AAGACTGATTCTGAATATATTGTTCTAGAGATATAA
Glycine max > GmLAZY4.3
SEQ ID NO: 15
MGFTFPLILQLEVVDIGKFFGTQKARLYGSKGLRNWRGEADDAKQEPREEFSDWPDGLLAIGT
FGNSNEVKEKTEKHILREDPSSSEEIADFTPEEIGKLQKELTKLLRQKPNVEKEIAELPLDRF
LNCPSSLEVDRRISNVLCSDSEDKDKDEEEREKEEEEDIEKTLSVILGKFKEICANNSKKAIG
KKSISFLLKKMFVCRSGFAPAPSLKDTLQLQESRMEKLLRIILHKKINSQHSSRALSLKKRLE
DRKMPKEDEAENDDGCKWWKTDSEYIVLEI
GmLAZY4.3
SEQ ID NO: 16
ATGAAGTTCCTCAGCTGGATGCAAAACAAAATTGGTGGAAAACAAGATAACAGAAAACCAAAC
ACATATACAACTACTCATGATGCAAAGCAAGAGCCTCGTGAAGAATTCAGCGATTGGCCTGAT
GGTTTACTAGCCATTGGTACATTTGGAAATAGCAATGAAGTAAAAGAAAAGACAGAGAAGCAC
ATTCTCAGAGAGGATCCATCCTCGTCAGAGGAAATAGCAGACTTCACTCCTGAAGAAATCGGG
AAACTACAAAAAGAGTTAACTAAACTGTTGAGACAAAAACCCAATGTGGAAAAGGAAATTGCT
GAGCTTCCTCTGGACAGATTTCTCAATTGTCCATCAAGCTTGGAGGTTGATAGGAGAATCAGT
AATGTACTTTGCAGTGATTCAGAAGACAAAGATAAAGATGAAGAAGAAAGAGAAAAAGAAGAA
GAAGAAGATATTGAAAAGACACTTAGTGTCATACTTGGTAAATTCAAAGAGATTTGTGCAAAT
AACAGCAAGAAAGCAATTGGGAAGAAATCAATTTCATTTTTGCTGAAGAAGATGTTTGTTTGT
AGAAGTGGATTTGCTCCAGCACCGAGCCTTAAAGACACCCTTCAGCTCCAAGAATCAAGAATG
GAGAAGCTTTTAAGGATAATTCTTCACAAGAAAATAAACTCCCAACATTCTTCTCGGGCATTG
TCCCTCAAGAAGCGCCTCGAGGACAGGAAGATGCCAAAGGAGGATGAAGCTGAAAATGATGAT
GGCTGTAAATGGGTCAAGACTGATTCTGAATATATTGTTTTAGAGATTTAA
Oilseed Rape
Brassica rapa BrLAZY4.1
SEQ ID NO: 17
MKLFGWMQNKLHGKQGNTHRPSTSSASSHQPREEFSDWPHGLLAIGTFGSVTKEQIPIETVQE
EKPSNLHVEGQAQDRDQDLSSSGDLEDFTPEEVGKLQKELTKLLTRKNKKRQSDVNRELANLP
LDRFLNCPSSLEVDRRISNALSGGCGDCDENEEDIERTISVILGRCKAISTESNSKKKKTKKD
LSKTSVSYLLKKMFVCTEGFSPLPKPSVRDTFQESRMEKLLRVMLLKKINAQAPSKETPTNRY
VQDKQQLSLKNEEEEGSSSSDGCKWVKTDSDFIVLEI
BrLAZY4.1
SEQ ID NO: 18
ATGAAGCTCTTTGGATGGATGCAGAACAAGCTACATGGGAAACAAGGGAACACTCATAGACCA
AGCACATCCTCTGCTTCTTCTCATCAACCACGAGAGGAGTTCAGCGACTGGCCTCATGGATTA
CTTGCGATTGGAACGTTCGGTAGTGTGACTAAAGAGCAAATACCAATAGAGACTGTTCAAGAA
GAGAAGCCCTCTAACTTGCACGTGGAAGGTCAAGCGCAAGATAGAGATCAAGATCTTTCCTCC
TCCGGTGATTTAGAAGATTTCACTCCAGAGGAAGTTGGGAAACTGCAAAAGGAGCTGACGAAG
CTCTTGACAAGAAAGAACAAGAAGAGACAGTCTGATGTGAACAGAGAACTTGCGAATCTTCCT
CTGGATAGATTCTTGAATTGTCCTTCGAGTCTTGAAGTCGATAGACGAATCAGCAACGCTCTT
TCTGGTGGTTGTGGAGATTGTGATGAGAACGAAGAAGACATTGAGCGTACAATCAGTGTTATC
TTGGGAAGATGCAAAGCCATTTCTACAGAGAGTAACAGTAAGAAGAAGAAGACTAAGAAAGAT
TTGAGCAAAACCTCTGTCTCTTATCTCCTCAAGAAGATGTTTGTCTGTACAGAAGGGTTCTCT
CCTCTTCCTAAACCTAGCGTGAGAGACACGTTTCAAGAATCAAGAATGGAAAAGTTACTGAGG
GTGATGCTACTCAAGAAGATTAATGCTCAAGCTCCCTCGAAGGAAACACCAACGAATAGATAC
GTGCAAGACAAGCAACAGCTTTCATTAAAGAATGAGGAAGAAGAAGGAAGTAGTAGTAGCGAT
GGGTGTAAATGGGTCAAAACAGATTCTGATTTCATTGTTCTTGAGATCTGA
Brassica rapa uncharacterized LOC103830789
(LOC103830789), mRNA
BrLAZY4.2
SEQ ID NO: 19
MKFFGWMQNKLHGKQGNTHRPSISSASSHQPREEFSDWPQGLLAIGTFGSVAKEQTQIQVVQE
VIQEENPSNVHVEGQVQDEDQDLSFSGDLEDFTPEEVGKLQKELTKLLTRKTKKRKSDVNREL
ANLPLDRFLNCPSSLEVDRRISNAISSGGYSNENEEDIERTISVILGRCKAISTESSNKKKKS
KRDMSKTSVSYLLKKMFVCSGGFSPLPNPSLRDTFQESRMEKLLRVMLHKKINAQAPSKETST
KRYVEDKQQLALKNEEEEGRSSDGSKWKTDSDFIVLEI
BrLAZY4.2
SEQ ID NO: 20
ATGCAGAACAAGCTACATGGGAAACAAGGGAACACTCATAGACCAAGCATATCTTCTGCTTCT
TCTCATCAACCAAGAGAGGAGTTCAGCGACTGGCCTCAAGGATTACTTGCGATTGGAACTTTC
GGTAGTGTGGCCAAAGAGCAAACACAAATACAAGTTGTTCAAGAAGTGATTCAAGAGGAGAAT
CCCTCTAACGTGCACGTGGAAGGTCAAGTTCAAGATGAAGATCAGGATCTTTCTTTCTCCGGT
GATCTTGAAGATTTTACTCCCGAGGAAGTTGGGAAACTGCAAAAGGAACTGACGAAGCTCTTG
ACAAGAAAGACCAAGAAAAGGAAGTCAGATGTGAACAGAGAACTTGCGAATCTTCCCCTGGAT
AGATTCTTGAATTGTCCTTCGAGTCTTGAAGTCGACAGACGAATCAGCAACGCGATTTCTAGT
GGTGGATATTCTAACGAGAACGAAGAAGACATTGAACGTACCATCAGTGTTATCTTGGGAAGA
TGCAAAGCTATTTCTACAGAGAGTAGCAATAAAAAGAAGAAGAGTAAGAGAGATATGAGCAAA
ACCTCTGTTTCTTATCTTCTCAAGAAGATGTTTGTTTGTTCAGGAGGGTTCTCTCCTCTTCCT
AACCCTAGCTTGAGAGACACGTTTCAAGAATCTAGAATGGAAAAGTTACTGAGGGTGATGCTA
CACAAGAAGATTAATGCTCAAGCTCCCTCGAAGGAAACATCAACAAAAAGATACGTGGAAGAT
AAGCAACAGCTTGCACTAAAGAACGAGGAAGAAGAAGGAAGAAGTAGTGATGGGAGCAAATGG
GTTAAAACAGATTCTGATTGTGAGTTTCAGATCTTTTGGTTTCTTAAATTTTTTTTTGAAAAA
AATGTTCAAGAATTGATTAGATCTTCTTCTTTGTTTTGGTTGCAGTCATTGTTCTTGAGATCT
GATCCCATTTTCCATTCTTCATGTTACAGGTAA
Brassica rapa BrLAZY4.3
SEQ ID NO: 21
MKLFGWMHNKLHGKQANTHRPRTSSACSHQSREEFSDWPHGLLAIGTFGTLIKDQTPIHVVQE
VIQEEKTSNMHVEGKAQDRNHDLSLSDDLEDFTPEEVGKLQNELTKLLTRKNKKRKSDVNKEL
ENLPLDRFLNCPSSFEVDRRISNAFSGGGDSDENQEDIERAISTILGRCKAISTGSKSKMKAK
RDWSKTSVSYLLKKMFVCTEGHSPLPNPGLRDTFQESRMEKFLRVMLLKKINTRACPKETSTC
RYVQDRQQLSLKNKEEEGRSSSDGSTWWKTDSDFIVLEI
BrLAZY4.3
SEQ ID NO: 22
ATGCATAATAAGCTACATGGTAAACAAGCGAATACTCATAGACCAAGAACATCATCTGCTTGT
TCTCATCAATCACGAGAAGAGTTCAGTGATTGGCCTCACGGATTACTTGCCATTGGAACGTTC
GGTACCTTGATCAAAGATCAAACCCCAATACATGTTGTTCAAGAAGTGATTCAAGAAGAGAAG
ACTTCTAACATGCACGTGGAAGGTAAAGCGCAAGATAGAAATCACGATCTTTCTTTATCCGAT
GATCTTGAAGATTTTACTCCCGAGGAAGTTGGGAAACTACAAAATGAGCTGACGAAGCTCTTG
ACAAGAAAGAACAAGAAGAGGAAGTCTGATGTGAACAAAGAACTTGAGAATCTTCCTTTGGAT
AGATTCTTGAATTGTCCTTCGAGTTTTGAAGTCGATAGACGAATCAGCAACGCGTTTTCAGGT
GGTGGAGATTCTGATGAGAACCAAGAAGACATTGAGCGTGCGATTAGTACTATTTTGGGGAGA
TGCAAAGCTATTTCTACAGGGAGTAAAAGTAAGATGAAGGCTAAGAGAGATTGGAGCAAAACC
TCTGTTTCTTATCTCCTCAAGAAGATGTTTGTATGTACAGAGGGGCACTCTCCTCTTCCTAAC
CCTGGCTTGAGAGACACGTTTCAAGAATCGAGAATGGAGAAGTTTCTGAGAGTAATGCTACTC
AAGAAGATTAATACTCGAGCTTGTCCAAAGGAAACATCAACGTGTAGATACGTGCAAGACAGG
CAACAACTTTCATTAAAGAATAAGGAAGAAGAAGGAAGAAGTAGTAGCGATGGGAGTACATGG
GTCAAAACAGATTCTGACTGTGAGTTTAAAATCTTTTTATTTCTTTTCAAAACAAAAGAAGTC
GTCCATGAACTAATTCTATTTTCATCATCTTCTTTTTGGTTGCAGTCATTGTTCTTGAGATCT
GATTCACTTTACCCCTACTCAGATTCTTACAGGAAAGTACAGGTAATATAG
Barley
Hordeum vulgare subsp. vulgare
SEQ ID NO: 23
MGIINWQNRLNTKQEKKRSAAGAAAASSARNAPDWEKSCRGQADDELPGDWSMLSIGTLGNEP
TPAPAPDQAVPDFTIEEVKKLQDALNKLLRRAKSKSSSRGSTAGAGDEEQNLPLDRFLNCPSS
LEVDRRLSLRLQAADGGQNGEFSPDTQIILSKARELLVSTNGNGGGVKQKSFKFLLKNMFACR
GGFPPQPSLKDPVETKLEKLFKTMLQKKMSVPRPSNAASSSRKYYLEDKPMGRIHMDGSHEEE
EDYNVEDIFKWDKTDSDCKSLELINFTAALTN
HvLAZY4
SEQ ID NO: 24
ATGGGGATCATCAACTGGGTGCAGAACCGCCTCAACACCAAGCAGGAGAAGAAACGATCGGCC
GCCGGCGCCGCTGCCGCCAGCTCGGCTCGCAATGCCCCGGACTGGGAGAAGAGTTGCCGCGGC
CAGGCCGACGACGAGCTCCCCGGCGACTGGAGCATGCTCTCCATCGGAACCCTCGGCAACGAG
CCCACGCCGGCGCCGGCGCCAGATCAGGCTGTGCCGGACTTCACCATCGAGGAGGTGAAGAAG
CTGCAGGACGCGCTGAACAAGCTACTCCGGCGCGCCAAGTCCAAGTCCAGCTCCCGCGGCTCC
ACCGCCGGCGCCGGCGACGAGGAACAGAACCTGCCGCTCGACAGGTTCCTCAACTGCCCCTCC
AGCCTCGAGGTCGACCGGCGGCTCTCGCTCAGGCTGCAAGCCGCCGACGGGGGACAGAACGGG
GAGTTCTCGCCTGACACGCAGATCATACTCAGCAAGGCCAGGGAGCTCCTCGTCAGCACCAAC
GGCAATGGCGGGGGCGTCAAGCAGAAGTCCTTCAAGTTCCTCCTCAAGAACATGTTCGCCTGC
CGGGGCGGCTTCCCGCCGCAGCCCAGCCTCAAGGATCCAGTTGAAACAAAATTGGAGAAGTTG
TTTAAGACGATGCTTCAAAAGAAGATGAGCGTCCCTCGCCCGAGCAACGCGGCATCGTCGTCG
AGGAAGTATTACCTAGAGGATAAACCAATGGGGAGGATCCACATGGATGGTAGCCACGAGGAG
GAGGAGGATTACAATGTTGAAGATATCTTCAAGTGGGACAAAACCGATTCAGATTGTAAGTCG
CTAGAGTTGATAAATTTCACTGCTGCCTTAACAAATTAA
Rice (Japonica)
Oryza sativa subsp. japonica
SEQ ID NO: 25
MGIINWMQNRLSTAKQDKRRTEAAAVASSARRRGGGGGESCRQEEARDEIKIAGDHLLSIGTL
GNESPPRPPAAAAATAAEEVADFTIEEVKKLQEALNKLLRRAKSTKSGSRRGSTAAEHDADER
SSSSSSSGSQLLLPLDRFLNCPSSLEVDRRVAAADGEFSPDTQIILSKARDLLVNTNGGGAIK
QKSFRFLLKKMFVCRGGFSPSPAPPPTLKDPVESRIEKLFRTMLHKRMNARPSNAAASSSRKY
YLEDKPREKMQREHLHDDEDDDENAEDIFKWDKTDSDFIVLEM
Os(Japonica)LAZY4
SEQ ID NO: 26
ATGGGGATTATTAACTGGATGCAGAATCGACTCAGTACTGCTAAACAAGACAAGAGACGAACT
GAAGCTGCTGCTGTGGCCTCGTCAGCTCGCAGACGAGGAGGAGGGGGAGGAGAGAGTTGCCGC
CAAGAAGAAGCTCGCGACGAGATCAAGATCGCCGGAGATCACCTCCTCTCCATCGGCACGCTC
GGGAACGAGTCGCCGCCGCGACCGCCGGCGGCGGCGGCGGCGACGGCGGCAGAGGAGGTGGCG
GACTTCACCATCGAGGAGGTGAAGAAGCTGCAGGAGGCGCTGAACAAGCTGCTCCGGCGAGCC
AAGTCCACCAAGTCCGGCAGCCGCCGCGGCTCGACGGCGGCGGAGCACGACGCCGACGAGCGC
TCCTCCTCCTCCTCCTCCTCCGGCAGCCAGCTGCTGCTGCCGCTCGACAGGTTCCTCAACTGC
CCCTCCAGCCTCGAGGTCGACCGGCGCGTGGCGGCGGCCGACGGCGAGTTCTCGCCGGACACG
CAGATCATCCTCAGCAAGGCGCGCGACCTCCTCGTCAACACCAATGGCGGCGGCGCCATCAAG
CAGAAATCCTTCAGGTTCCTCCTCAAGAAGATGTTCGTCTGCCGCGGCGGCTTCTCGCCGTCG
CCGGCGCCGCCGCCCACCTTGAAGGATCCAGTCGAATCAAGAATCGAAAAGTTGTTCAGGACG
ATGCTTCACAAGAGGATGAACGCTCGACCGAGTAATGCTGCGGCGTCGTCGTCGAGGAAATAC
TATCTTGAGGATAAGCCGAGGGAGAAGATGCAAAGGGAGCATCTCCATGATGATGAAGATGAT
GATGAGAATGCAGAAGATATCTTTAAATGGGACAAAACTGATTCAGATTTCATTGTTCTGGAG
ATGTAG
Rice (Indica)
Oryza sativa subsp. indica
SEQ ID NO: 27
MGIINWMQNRLSTAKQDKRRTEAAAVASSARRRGGGGGESCRQEEARDEIKIAGDHLLSIGTL
GNESPPRPPPAAAATAAEEVADFTIEEVKKLQEALNKLLRRAKSTKSGSRRGSTAAEHDADER
SSSSSSSGGQLLLPLDRFLNCPSSLEVDRRVAAADGEFSPDTQIILSKARDLLVNTNGGGAIK
QKSFRFLLKKMFVCRGGFSPSPAPPPTLKDPVESRIEKLFRTMLHKRMNARPSNAAASSSRKY
YLEDKPGEKMQREHLHDDEDDDENAEDIFKWDKTDSDCNHCSGDVDRDARFNAIIIVCTMISD
TVGVRFTI
Os(Indica)LAZY4
SEQ ID NO: 28
ATGGGGATTATTAACTGGATGCAGAATCGACTCAGTACTGCTAAACAAGACAAGAGACGAACT
GAAGCTGCTGCTGTGGCCTCGTCAGCTCGCAGACGAGGAGGAGGGGGAGGAGAGAGTTGCCGC
CAAGAAGAAGCTCGCGACGAGATCAAGATCGCCGGAGATCACCTCCTCTCCATCGGCACGCTC
GGGAACGAGTCGCCGCCGCGACCGCCGCCGGCGGCGGCGGCGACGGCGGCAGAGGAGGTGGCG
GACTTCACCATCGAGGAGGTGAAGAAGCTGCAGGAGGCGCTGAACAAGCTGCTCCGGCGAGCC
AAGTCCACCAAGTCCGGCAGCCGCCGCGGCTCGACGGCGGCGGAGCACGACGCCGACGAGCGC
TCCTCCTCCTCCTCCTCCTCCGGCGGCCAGCTGCTGCTGCCGCTCGACAGGTTCCTCAACTGC
CCCTCCAGCCTCGAGGTCGACCGGCGCGTGGCGGCGGCCGACGGCGAGTTCTCGCCGGACACG
CAGATCATCCTCAGCAAGGCGCGCGACCTCCTCGTCAACACCAATGGCGGCGGCGCCATCAAG
CAGAAATCCTTCAGGTTCCTCCTCAAGAAGATGTTCGTCTGCCGCGGCGGCTTCTCGCCGTCG
CCGGCGCCGCCGCCCACCTTGAAGGATCCAGTCGAATCAAGAATCGAAAAGTTGTTCAGGACG
ATGCTTCACAAGAGGATGAACGCTCGACCGAGTAATGCTGCGGCGTCGTCGTCGAGGAAATAC
TATCTTGAGGATAAGCCGGGGGAGAAGATGCAAAGGGAGCATCTCCATGATGATGAAGATGAT
GATGAGAATGCAGAAGATATCTTTAAATGGGACAAAACTGATTCAGATTGTAATCATTGTTCT
GGAGATGTAGACCGAGACGCACGATTCAATGCGATCATTATTGTTTGCACAATGATTTCAGAT
ACAGTTGGTGTACGTTTCACCATATAG
Oryza sativa subsp. indica
SEQ ID NO: 29
MGIVSWVQGRLGGRTSAAAESRGLAAGNGNPSLVAAVVAPGKERKHQQVVPDDLAGDQWPTPA
THLFSIGTLGNDELPEQGEEEEDLPEFSVEEVRKLQDALARLLLRARSKNYSEAVATAAATAT
CCGGGGADSGLPLDMFLNCPSSLEVDRRAQRDHGGGGAAVGLSPGTKMILTKAKDILVDGNTR
NTTTSGGDIKNKSFKFLLKKMFVCHGGFAPAPSLKDPTESSMEKFLRTVLGKKIAARPSNSPA
SRTYFLEGNNAHGDDHRLCRRRRPRCGEEEEEEEENKGEESCKWDRTDSEYIVLEI
Os(Indica)LAZY4.2
SEQ ID NO: 30
ATGGGGATCGTCAGCTGGGTGCAGGGGAGGCTGGGTGGGAGGACGTCGGCGGCGGCGGAGAGC
AGAGGGCTCGCCGCCGGCAACGGCAATCCTTCGCTGGTCGCGGCGGTCGTTGCGCCAGGCAAG
GAGAGGAAGCATCAGCAGGTTGTTCCTGACGATCTCGCCGGCGATCAATGGCCGACTCCGGCG
ACTCATCTCTTCTCCATCGGCACGTTGGGCAACGACGAGTTGCCGGAGCAGGGGGAGGAGGAG
GAGGACCTGCCGGAGTTCAGCGTCGAGGAGGTGAGGAAGCTCCAGGACGCGCTGGCGAGGCTC
CTCCTGCGCGCCAGGTCCAAGAATTATTCCGAGGCCGTCGCCACCGCCGCCGCCACCGCCACC
TGCTGCGGCGGCGGCGGCGCGGACAGTGGCCTGCCGCTCGACATGTTCCTCAACTGCCCTTCC
AGCCTCGAGGTGGACAGGAGAGCACAGCGCGATCACGGCGGCGGAGGCGCCGCCGTCGGCCTC
TCGCCGGGCACCAAGATGATACTCACCAAGGCCAAGGACATTCTCGTCGACGGCAACACCAGA
AACACCACCACCAGCGGCGGCGACATCAAGAACAAGTCATTCAAGTTCCTTCTCAAGAAGATG
TTCGTCTGCCATGGCGGCTTCGCGCCGGCTCCGAGCTTGAAGGACCCGACGGAATCATCAATG
GAGAAGTTTCTCCGAACGGTGCTCGGCAAGAAGATCGCTGCCCGGCCGAGCAATTCACCGGCG
TCGAGGACATACTTCTTGGAGGGTAACAATGCACATGGTGATGACCATCGCCTTTGTCGCCGC
CGTCGTCCTCGTTGCGGCGAAGAAGAAGAAGAGGAGGAGGAGAACAAGGGGGAAGAAAGTTGT
AAATGGGACAGGACAGATTCTGAATATATTGTTCTTGAGATATGA
Sorghum
Sorghum bicolor
SEQ ID NO: 31
MGIINWMQNRFNGKHEKRRPEATAAAAAAAFSSAHESCRQDHGREDKIPTGDWPPQGLLSIGT
LGDDPPPAAGDGGGGPPRASQADVLDFTIEEVKKLQDALNKLLRRAKSKSSSSRGSGATDEDR
ASQLPLDRFLNCPSSLEVDRRISLRHAAGDGGGENGEFSPDTQIILSKARDLLVNSNGTTIKK
KSFKFLLKKMFVCHGGFAPAPSLKDPVESRIEKLFRTMLQKKMNNARPSNAAVSSRKYYLEDK
PSGRMMIRDGHHDEEDDEKGSDRIKWDKTDTDFIVLEI
SbLAZY4.1
SEQ ID NO: 32
ATGGGGATCATTAACTGGATGCAGAATCGCTTCAATGGTAAACATGAGAAGAGGCGACCCGAG
GCCACCGCCGCCGCCGCCGCCGCCGCCTTTAGCTCAGCTCACGAAAGCTGCCGCCAAGACCAC
GGTCGCGAGGACAAGATCCCCACCGGCGACTGGCCGCCACAGGGCCTCCTCTCGATCGGGACA
CTGGGCGACGACCCACCACCGGCGGCGGGAGATGGAGGTGGAGGCCCGCCGCGCGCGTCGCAG
GCCGATGTGCTGGACTTCACCATCGAGGAGGTGAAGAAGCTGCAGGACGCGCTGAACAAGCTG
CTCCGGCGCGCCAAGTCCAAGTCCAGCTCCTCCCGCGGGTCGGGCGCCACCGACGAGGACCGC
GCTAGCCAGCTGCCGCTCGACAGGTTCCTCAACTGCCCATCCAGCCTCGAGGTCGACCGGAGG
ATCTCCCTGAGGCACGCCGCCGGCGACGGTGGTGGCGAGAATGGCGAGTTCTCGCCAGACACG
CAGATCATACTCAGCAAGGCCAGGGATCTCCTCGTTAACAGTAACGGCACCACCATCAAGAAG
AAGTCGTTCAAGTTCCTCCTCAAGAAGATGTTCGTCTGCCATGGCGGCTTCGCCCCCGCACCG
AGCTTGAAGGATCCAGTTGAATCAAGGATAGAGAAGTTGTTCAGAACGATGCTTCAGAAGAAG
ATGAACAATGCTCGCCCGAGCAATGCTGCAGTGTCATCCAGGAAGTACTACCTCGAAGACAAA
CCGAGTGGGAGGATGATGATACGGGATGGGCATCACGATGAAGAGGATGATGAAAAGGGTTCT
GACAGAATCAAGTGGGATAAAACTGATACTGACTTCATTGTTCTGGAGATCTAA
Sorghum bicolor
SEQ ID NO: 33
MGIINWMQNRFHGKTENRIFDGGATATSSYRGAGAQERQETIIREPEKHLDAEPWPQAPAGLL
SIGTLGSEEPPPPAAQDLPEFTVEEVKKLQDALAMLLRRAKSKSSARGSAAGEDRPPLDRFLN
CPSCLEVDRRVQTTAKHGECGGGQEGEGDLSPDTKIILTRARDLLDSGGGIKQRSFKFLLKKM
FACNGGFSAAPPRSLKDPVESRMEKFFRTVIGKKMNASSGNRSSTSRKYFLEDGTSKGKRRGA
RRCGCQEEEEEREESCKWDRTDSEFIVLEI
SbLAZY4.2
SEQ ID NO: 34
ATGGGGATCATCAACTGGATGCAGAACAGATTCCATGGGAAGACCGAGAACAGAATCTTTGAC
GGCGGCGCAACTGCCACCAGTTCATATAGAGGCGCTGGAGCCCAAGAGAGACAAGAGACGATC
ATTCGTGAACCAGAGAAGCATCTCGACGCCGAGCCATGGCCTCAGGCGCCGGCGGGGCTCCTC
TCCATCGGCACGCTCGGCAGCGAGGAGCCTCCGCCGCCGGCAGCGCAGGACCTGCCGGAGTTC
ACCGTGGAGGAGGTGAAGAAGCTCCAGGACGCGCTGGCCATGCTCCTGCGGCGCGCCAAGTCC
AAGTCCAGCGCCCGCGGCTCCGCGGCCGGCGAGGACAGGCCGCCGCTGGACAGGTTCCTCAAC
TGCCCGTCCTGCCTGGAGGTGGACAGGCGGGTCCAGACGACGGCCAAGCACGGCGAGTGCGGC
GGTGGCCAGGAAGGCGAAGGAGACCTCTCGCCGGACACCAAGATCATACTGACCAGGGCCAGA
GACCTGCTCGACAGCGGCGGCGGCATCAAGCAGAGGTCGTTCAAGTTCCTGCTCAAGAAGATG
TTCGCCTGCAATGGCGGCTTCTCGGCGGCGCCGCCTCGGAGCTTGAAGGACCCAGTGGAGTCA
AGAATGGAGAAGTTCTTCCGAACGGTGATCGGGAAGAAGATGAATGCCAGCTCGGGCAACAGG
TCGTCAACGTCGAGGAAGTACTTCTTGGAGGATGGAACCAGCAAGGGGAAGAGGCGAGGTGCT
CGTCGTTGTGGTTGCCAAGAGGAGGAGGAGGAGAGGGAAGAGAGCTGCAAATGGGACAGAACA
GATTCTGAATTCATTGTTTTGGAGATATGA
Cotton
Gossypium raimondii
SEQ ID NO: 35
MKFFGWWQNKLNGKPGRSKPQTDSATNYMKQEPRQEFSDWPHGLLAIGTFGNNNDMIENPPSQ
NTARQDPFDIREEHEPSSSEDLHEFTPEEVGKLEKELTKLLSRKPASDVKKELANLPLDRFLN
CPSSLEVDRRISNAVCSDSGDKSDQEDIDRTISVILGRCKDICAEKNKKSIGKKSLSFLLKKM
FACGSGFSPAPSLRDVLQESKMERLLRVMLHKKIYNQNPSGASAVKKYLEDRQSPKRRNKLNN
EDETQERKSEDGYKWWKTDSEYIVLEI
GrLAZY4.1
SEQ ID NO: 36
ATGAAATTCTTTGGTTGGGTCCAAAATAAGCTTAATGGGAAACCGGGGCGCAGTAAACCACAA
ACAGATTCTGCTACTAATTACATGAAACAGGAGCCTCGACAAGAGTTCAGCGATTGGCCTCAT
GGATTGTTGGCTATAGGAACGTTTGGCAACAATAATGACATGATAGAAAATCCTCCATCCCAA
AACACCGCCCGACAAGATCCGTTTGATATTCGCGAGGAACACGAGCCGTCCTCATCGGAGGAT
TTACACGAATTTACGCCCGAAGAAGTCGGGAAACTAGAAAAGGAATTAACCAAACTCTTGTCC
CGAAAACCGGCTTCCGATGTTAAAAAGGAACTAGCAAATCTACCATTGGATAGGTTTCTTAAC
TGTCCATCGAGCTTGGAAGTTGATAGGAGGATTAGCAATGCGGTTTGTAGTGATTCAGGGGAT
AAATCAGATCAAGAAGACATTGATCGAACCATTAGTGTTATTCTCGGCCGATGCAAAGACATT
TGCGCTGAAAAAAACAAGAAATCCATCGGCAAAAAATCGCTTTCTTTCCTTTTGAAGAAGATG
TTTGCTTGCGGCAGTGGATTTTCACCTGCCCCGAGCTTGAGAGATGTGCTGCAAGAATCGAAA
ATGGAGAGGCTTTTGAGGGTAATGCTTCACAAGAAGATTTACAATCAGAACCCTTCTGGAGCA
TCAGCTGTGAAGAAATATTTAGAAGACAGACAGTCTCCGAAAAGGCGAAATAAATTAAATAAT
GAAGATGAAACCCAGGAGAGGAAGAGTGAAGATGGATATAAATGGGTGAAGACAGATTCTGAA
TATATTGTTCTGGAGATCTAA
Gossypium raimondii
SEQ ID NO: 37
MKFFGWMQNKLNGKQGPSKSNTISATYHMKQEPREEFSDWPHGLLAIGTFGNNELKENPESQS
TIQQEPIEIQDQEPCSSDDLQEFTVEEVGKLQKELTKLLSRKPNPNTKKEVASLPLDRFLNCP
SSLEVDRRFSNAVCSDAGERSEEDIDRTISIILGRCKDIRGEDNKKKAIGKKSISFLLKKMFV
CSGGFPPTPTLRDTLQESRMEKLLRVMLHKKIYSQNPTREPSMKKYLEDKQTPKRQKIPDENE
TVERKSEDGGKWVKTDSEYIVLEI
GrLAZY4.2 (B456_011G061600)
SEQ ID NO: 38
ATGAAGTTCTTTGGTTGGATGCAAAATAAGCTTAATGGGAAACAAGGACCCAGCAAGTCAAAT
ACAATATCTGCTACTTATCATATGAAACAAGAGCCTCGGGAGGAGTTCAGTGATTGGCCACAT
GGACTGTTAGCAATAGGGACATTTGGTAACAATGAGCTTAAAGAAAACCCTGAATCCCAAAGC
ACCATTCAACAGGAACCCATTGAGATTCAAGACCAAGAGCCATGTTCGTCCGATGATTTACAG
GAGTTCACGGTCGAAGAAGTCGGGAAACTACAAAAGGAACTAACGAAACTCTTGTCCCGAAAA
CCGAACCCCAACACAAAAAAAGAAGTAGCAAGTTTACCATTGGATAGATTTCTTAATTGTCCA
TCAAGCTTGGAAGTGGATAGAAGGTTTAGCAATGCGGTTTGCAGTGATGCAGGGGAGAGATCG
GAGGAAGACATCGATCGAACCATTAGCATTATCCTCGGCAGATGCAAAGACATACGTGGTGAA
GATAATAAGAAAAAGGCCATTGGGAAGAAATCAATTTCTTTCCTTTTGAAGAAGATGTTTGTT
TGTTCAGGTGGATTTCCACCTACACCAACTTTGAGAGATACACTACAAGAATCAAGAATGGAG
AAGCTTTTGAGGGTAATGCTTCACAAGAAGATTTACAGTCAAAATCCAACTAGAGAACCATCA
ATGAAGAAATACTTGGAGGACAAGCAAACACCCAAAAGGCAAAAAATTCCAGATGAAAATGAA
ACAGTGGAGAGAAAGAGTGAAGATGGAGGTAAATGGGTGAAAACAGATTCTGAATATATTGTT
CTAGAGATATAA
Nicotiana
Nicotiana attenuata
SEQ ID NO: 39
LQFFSWMQNKFNGGQGNRSMPNEVQTKKRPRNEEFNGWPDSLLAIGTFGTSSSNLKAKSESQN
VQNQERDEIILDDNINEQSSSPDLAEFTPEEVGKLQKELTKLLSKKPAAKLIDQGRQDGDLPL
DRFLNCPSSLEVDRRASSSRFSSTNYSDNYDNYDEEEIDRTIRAIIGRCKDHVCKTNKKKVNG
MKSISFLLKKMFVCSSGFAPTPSLRDTFPESRMEKLLRTILSKKIINPQNAARVSTKRYLEDR
CVPKEEEEEKKREKTCDGSKWVKTDSD
NaLAZY4.1
SEQ ID NO: 40
TTGCAGTTCTTTAGCTGGATGCAAAATAAGTTCAATGGCGGACAAGGGAACAGATCAATGCCT
AATGAAGTTCAAACCAAAAAACGTCCTCGCAACGAAGAATTCAACGGTTGGCCTGATTCGTTA
TTAGCCATTGGAACTTTTGGTACCAGCAGCAGTAATCTCAAAGCAAAATCAGAGAGCCAAAAC
GTACAAAATCAAGAACGGGATGAAATAATCTTAGATGATAATATTAATGAGCAAAGTTCCTCT
CCAGATTTAGCAGAATTCACACCTGAAGAAGTTGGTAAATTACAGAAAGAATTAACAAAGTTA
TTATCAAAAAAACCAGCTGCTAAATTAATTGATCAAGGACGACAAGATGGTGATCTCCCATTG
GATAGATTCCTTAATTGCCCTTCAAGTTTAGAAGTGGATCGTAGGGCTTCTTCCAGCAGATTT
AGCAGTACTAATTACTCAGATAATTATGATAATTATGATGAGGAAGAAATTGATAGAACTATT
AGAGCAATCATTGGAAGATGCAAGGATCATGTTTGCAAGACAAATAAAAAGAAAGTAAATGGG
ATGAAATCCATTTCTTTCCTTCTCAAGAAAATGTTTGTTTGCTCAAGTGGTTTTGCTCCTACT
CCTAGTTTACGAGATACATTTCCAGAATCAAGAATGGAGAAGCTTTTAAGGACAATACTTTCC
AAGAAAATAATAAACCCTCAAAATGCAGCTCGAGTATCAACAAAGAGATACTTAGAGGACCGA
TGTGTACCAAAGGAAGAGGAAGAGGAGAAAAAACGGGAGAAAACTTGTGATGGATCTAAGTGG
GTGAAGACTGATTCTGAT
Nicotiana attenuata
SEQ ID NO: 41
CPQITNFANVNSRFILDMKFFNWMHNKLNGGQGSKKPNAVPITNQTNEEFKDWPDSLLAIGTF
GNKSSDLEESRPKTHVQNDHHHEDEILENSPDLAEFTPEEVGKLQKELTKLLSRKPADDILPL
DRFLNCPSSLEVDRRISSSSTNSDNFDYDEEEIDRTIRVIIGRCKDVCSKQNKKKAIGKKSIS
FLLKKMFACASGNFGPPPTFPDPFHESRMEKLLRTMLSKKINPQNASRTSTKRYLEDKQPKKE
EQEEKKREKTCNDGSKWWKTDSEFIVLEM
NaLAZY4.2
SEQ ID NO: 42
TGTCCACAAATTACCAACTTCGCAAACGTCAACAGCAGATTCATTTTAGATATGAAGTTCTTT
AACTGGATGCATAATAAGTTAAATGGGGGACAAGGAAGCAAAAAACCTAATGCAGTTCCTATC
ACAAATCAAACAAATGAAGAGTTTAAAGATTGGCCAGATTCGTTATTGGCAATTGGAACTTTT
GGCAACAAGAGCAGTGATCTCGAAGAAAGTAGACCAAAAACACACGTACAAAATGATCATCAT
CACGAGGACGAAATCCTAGAGAATTCACCAGATTTAGCAGAATTCACACCTGAAGAAGTTGGC
AAATTACAAAAAGAATTAACAAAATTATTATCCCGAAAACCGGCTGATGATATTCTTCCATTG
GACAGATTTCTTAATTGTCCGTCAAGTTTGGAAGTTGATCGCAGGATTAGTTCCAGCAGTACT
AATTCAGACAATTTTGATTATGACGAGGAAGAAATTGACAGAACTATAAGAGTGATTATAGGA
AGATGCAAAGATGTCTGTAGTAAGCAGAACAAAAAGAAAGCAATTGGGAAGAAATCTATTTCT
TTTCTTCTCAAGAAAATGTTCGCTTGTGCAAGTGGTAATTTTGGTCCACCTCCTACTTTCCCA
GATCCATTTCACGAATCAAGAATGGAGAAGCTTTTGAGGACAATGCTTTCCAAGAAAATAAAC
CCTCAAAATGCCTCTCGGACATCAACAAAGAGATATTTAGAGGACAAACAACCAAAAAAGGAA
GAGCAAGAAGAGAAAAAACGAGAGAAAACCTGTAATGATGGATCTAAATGGGTGAAAACTGAT
TCTGAATTTATCGTCTTGGAGATGTAG
Tomato
SEQ ID NO: 43
MKLFSWQNKFNGGQVNKVQTKNQPSKEPRNEEFNGWPDSLLAIGTFGASSSSLKPKIQNDNDN
DNEISEDVKQSSSPDLAEFTPEEVGKLQKELTKLLSKKPAAAAKLTAAAEGRQDGNLPLDRFL
NCPSSLEVDRRTSSRFSSTNSEIYENLDEEEIDRTIRAIIGRLNGMKSVTFLLKKMFVCSSGF
APTPNLRDTLPESRMEKLLRTILSKKIIPQSASRISTKRYLEDRCVPKEEVEEKKRDKTCDGS
KWKTDSDFIVLEI
SILAZY4
SEQ ID NO: 44
ATGAAGTTCTTTAATTGGATGCATAACAAGCTCAATGGTGGACAAGGAAGTAGGAGGTCTAAT
GCTATGCCAATTACTACAAATCATAATATAAATGAAGAATTCAAAGATTGGCCAGATTCGTTG
TTATCAATTGGAACTTTTGGCAATAGAAGCAGTGATCTCAAAGAACAGAGCAAATTACACGTG
AAAGACGATGAACTAACTTCTTATTCTTCTTCTCCAGAATTAGCAGAATTCACGTCTGAAGAA
GTCGAGAAGTTACAGAAGGAGTTAACAAAGTTACTATCACGAAAACCACCCCCAACTGCTAGT
AATTCTGAGTTTGTTGACATCAAGAACGGCGCTGCCAATGCTGATGATATCCTTCCGTTGGAC
AGATTTCTTAATTGTCCATCGAGCTTGGAAGTTGATCGTAGGGTTAATTCCAGTAGATTTAGC
AGTGTTAATTACTCGTACGATTACGACGAGGAAGAAATCGACAGAACAATAAGAGTAATTATA
GGTAGATGCAAGGATGTTTGTAGAAAACAGAGCAAAAAGAAATCAATTGGGATGAAATCAATT
TCTTTCCTTCTCAAGAAAATGCTTGTTTGTACAAAGGGTGGTTTTGCTCCCGCTCCCAATTTA
CGTGACACATTTCCCGAATCAAGAATGGAGAAGCTTTTGAGGACAATGCTTTCCAAGAAAATA
CATCCCCAAAATGCCCCTCGAACATCAACAAAGAGATATTTAGAGGAAAAACATGCACAAAGA
GAAGAGAAAGTATCGTCTTGGAAATATAG
gRNA for wheat and barley
SEQ ID NO: 45
5′-TCGACCGGCGGCTCTCGCTC-3
Sequences for ZmLAZY4
PAM:
CCA
gRNA:
SEQ ID NO: 46
GCCTCGAGGTCGACCGGAGG
Change: R142Q
Sequences for GmLAZY4.1, GmLAZY4.2, GmLAZY4.3
PAM:
AGG
gRNA:
SEQ ID NO: 47
CTTCAAGCTTGGAGGTTGAT
Change: S (120, 141, 131 respectively) L
Sequences for BrLAZY4.1
PAM:
CCT
gRNA:
SEQ ID NO: 48
TCGAGTCTTGAAGTCGATAG
Change: V139I, D140N
Sequences for BrLAZY4.2
PAM:
CTT
gRNA:
SEQ ID NO: 49
TCGAGTCTTGAAGTCGACAG
Change: V143I, D144N
Sequences for OsLAZY4 (Japonica and Indica 1)
PAM:
CCA
gRNA:
SEQ ID NO: 50
GCCTCGAGGTCGACCGGCGC
Change: R155Q
Sequences for OsLAZY4.2 (Indica)
PAM:
CCA
gRNA:
SEQ ID NO: 51
GCCTCGAGGTGGACAGGAGA
Change: R153K
Sequences for SbLAZY4.1
PAM:
AGG
gRNA:
SEQ ID NO: 52
TCGACCGGAGGATCTCCCTG
Change: R146W
Sequences for SbLAZY4.2
PAM:
CCT
gRNA:
SEQ ID NO: 53
GCCTGGAGGTGGACAGGCGG
Change: R135K
Sequences for GrLAZY4.1
PAM:
AGG
gRNA:
SEQ ID NO: 54
CATCGAGCTTGGAAGTTGAT
Change: S129L
Sequences for GrLAZY4.2
PAM:
CCA
gRNA:
SEQ ID NO: 55
TCAAGCTTGGAAGTGGATAG
Change: V131I, D132N
Sequences for NaLAZY4.1
PAM:
CCT
gRNA:
SEQ ID NO: 56
TCAAGTTTAGAAGTGGATCG
Change: V138I, D139N
Sequences for NaLAZY4.2
PAM:
CCG
gRNA:
SEQ ID NO: 57
TCAAGTTTGGAAGTTGATCG
Change: V138I, D139N
Sequences for SILAZY4
PAM:
CCA
gRNA:
SEQ ID NO: 58
TCGAGCTTGGAAGTTGATCG
Change: V1351, D136N
Sequences for BoLAZY4.1, BoLAZY4.2 (
PAM:
CCT
gRNA:
SEQ ID NO: 59
TCGAGTCTTGAAGTCGATAG
Change: V(139/140 respectively)I, D(140/141 respectively)N
Sequences for BoLAZY4.23
PAM:
CCT
gRNA:
SEQ ID NO: 60
TCGAGTTTTGAAGTCGATAG
Change: V134I, D135N
Oilseed rape
Brassica Oleracea
SEQ ID NO: 61
MKLFGWMQNKLHGKQGNTHRPSTSSASSHQPREEFSDWPHGLLAIGTFGSVAKEQTPIETVQE
EKPSNVHVEGQAQDRDQDLSPSGDLEDFTPEEVGKLQKELTKLLTRKNKKRKSDVNRELANLP
LDRFLNCPSSLEVDRRISNALSGGGGDCDENEEDIERTISVILGRCKAISTESNSKKKKTKKD
LSKTSVSYLLKKMFVCTEGFSPLPKPILRDTFQESRMEKLLRVMLLKKINAQAPSKETPMKKY
VQDEQQLSLKNEEEEGSSSSSDGCKWWKTDSDFIVLEI
BoLAZY4.1
SEQ ID NO: 62
ATGAAGCTCTTTGGATGGATGCAGAACAAGCTACATGGGAAACAAGGGAACACTCATAGACCA
AGTACATCCTCTGCTTCTTCTCATCAACCACGAGAGGAGTTCAGCGACTGGCCTCATGGACTA
CTTGCGATTGGAACGTTCGGTAGTGTGGCCAAAGAGCAAACACCAATAGAGACTGTTCAAGAA
GAGAAGCCCTCTAACGTGCACGTGGAAGGTCAAGCGCAAGATAGAGATCAAGATCTTTCACCC
TCCGGTGACCTAGAAGATTTCACTCCGGAGGAAGTTGGGAAACTTCAGAAGGAGCTGACGAAG
CTCTTGACAAGAAAGAACAAGAAGAGGAAGTCCGATGTGAATAGAGAACTTGCGAATCTTCCT
CTGGATAGATTCTTGAATTGTCCTTCGAGTCTTGAAGTCGATAGACGAATCAGCAACGCTCTT
TCTGGTGGTGGTGGAGATTGTGATGAGAACGAAGAAGACATTGAGCGTACGATCAGTGTTATC
TTGGGAAGATGCAAAGCCATTTCTACAGAGAGTAACAGTAAGAAGAAGAAGACTAAGAAAGAT
TTGAGCAAAACCTCTGTCTCTTATCTCCTCAAGAAGATGTTTGTCTGTACAGAAGGGTTCTCT
CCTCTTCCTAAACCTATCTTGAGAGACACGTTTCAAGAATCAAGAATGGAAAAGTTACTGAGG
GTGATGCTACTCAAGAAGATTAATGCTCAAGCTCCCTCGAAGGAAACACCAATGAAGAAATAC
GTGCAAGACGAGCAACAGCTTTCACTAAAGAATGAGGAAGAAGAAGGAAGTAGTAGTAGTAGC
GATGGGTGTAAATGGGTCAAAACAGATTCTGATTTCATTGTTCTTGAGATCTGA
Brassica oleracea var. oleracea
SEQ ID NO: 63
MKLFGWMQNKLHGKQGNTHRPSISSASSHQPREEFSDWPQGLLAIGTFGSVAKEQTQIQVVQE
VFKEENPSDVNMEAHRDQDLSFSGDLDDFTPEEVGKLQKELTKLLTRKNKMRKSDVNRELANL
PLDRFLNCPSSLEVDRRISNALASGGDFDENEEEMERTISVILGRCKAISTESSNKKKKSKRD
LSKTSVFYLFKKMFVCSEGLSPLPNPSLRDTFQESRMEKLLRVMLHKKINAQASSKQTSTKRY
VEDKQQLSLKNEEEEGRSGDGSKWKTDSDFIVLEI
BoLAZY4.2
SEQ ID NO: 64
ATGAAGTTATTCGGATGGATGCAGAACAAGCTACATGGGAAACAAGGGAACACTCATAGACCA
AGCATATCTTCTGCTTCTTCTCATCAACCCAGAGAGGAGTTCAGCGACTGGCCTCAAGGATTA
CTTGCGATTGGAACTTTCGGTAGTGTGGCCAAAGAGCAAACACAAATACAAGTTGTTCAAGAA
GTGTTCAAAGAGGAGAATCCCTCTGACGTGAACATGGAAGCTCATAGAGATCAAGATCTTTCT
TTCTCCGGTGATCTTGATGATTTTACTCCCGAGGAAGTCGGGAAACTGCAAAAGGAACTGACC
AAGCTCTTGACAAGAAAGAACAAGATGAGGAAGTCTGATGTAAATAGAGAACTTGCGAATCTT
CCTTTGGATAGATTCTTGAACTGTCCTTCGAGTCTTGAAGTCGATAGACGAATCAGCAACGCG
CTCGCTAGTGGTGGTGATTTTGATGAGAACGAAGAAGAAATGGAGCGTACAATCAGTGTTATC
TTGGGAAGATGCAAAGCTATTTCTACAGAGAGCAGCAATAAAAAGAAGAAGAGTAAGAGAGAT
TTGAGCAAAACCTCTGTTTTTTATCTTTTCAAGAAGATGTTTGTATGTTCAGAGGGGTTATCT
CCTCTTCCCAACCCTAGCTTGAGAGACACGTTTCAAGAATCAAGAATGGAAAAGTTACTGAGG
GTGATGCTACACAAGAAGATTAATGCTCAAGCTTCCTCGAAGCAAACATCAACAAAGAGATAC
GTGGAAGATAAGCAACAGCTTTCACTAAAGAACGAGGAAGAAGAAGGAAGAAGTGGTGATGGG
AGCAAATGGGTTAAAACAGATTCTGATTTCATTGTTCTTGAGATCTGA
Brassica oleracea var. oleracea
SEQ ID NO: 65
MHNKLHGKQANTHKRRTSSACSHQSREEFSDWPHGLLAIGTFGTLTKDQTPIQEVIQEEKTSN
MHVEGRAQDRDHDISLSDDLEDFTPEEVGKLQNELTKLLTRKNKKRKSDVNKELANLPLDRFL
NCPSSFEVDRRISNAFSGGGDSDENQEDIERTISIILGRCKAIYTESKNKKKGKRDVSKTSVS
YLLKKMFFLRVMLLKKINTRASPKQTSTSRYVQDRQQLSLKNKEEEGRSSSSSDGSKWWKTDS
DCSYRKVQIENLH
BoLAZY4.3
SEQ ID NO: 66
ATGCATAATAAGCTACATGGTAAACAAGCGAATACTCATAAACGAAGAACATCATCTGCTTGT
TCTCATCAATCACGAGAAGAGTTCAGCGATTGGCCTCACGGATTACTTGCCATTGGAACGTTC
GGTACCTTGACCAAAGATCAAACCCCAATACAAGAAGTGATTCAAGAAGAGAAGACTTCTAAC
ATGCACGTGGAAGGTAGAGCGCAAGATAGAGATCACGATATTTCTTTATCCGATGATCTTGAA
GATTTTACTCCCGAGGAAGTTGGGAAACTACAAAATGAGCTGACGAAGCTCTTGACAAGAAAG
AACAAGAAGAGGAAGTCTGATGTGAACAAAGAACTTGCCAATCTTCCTTTGGATAGATTCTTG
AATTGTCCTTCGAGTTTTGAAGTCGATAGACGAATCAGCAACGCGTTTTCAGGTGGTGGAGAT
TCTGATGAGAACCAAGAAGACATTGAGCGTACGATTAGTATTATTTTGGGGAGATGCAAAGCT
ATTTATACAGAGAGTAAAAATAAGAAGAAGGGTAAGAGAGATGTGAGCAAAACCTCTGTTTCT
TATCTCCTCAAGAAGATGTTTTTTCTGAGAGTAATGCTACTCAAGAAGATTAATACTCGAGCT
TCTCCAAAGCAAACATCAACGAGTAGATACGTGCAAGACAGGCAACAACTTTCATTAAAGAAT
AAGGAAGAAGAAGGAAGAAGTAGTAGTAGTAGCGATGGGAGTAAATGGGTCAAAACAGATTCT
GATTGTTCTTACAGGAAAGTACAGATAGAGAATCTTCATTGA
Wheat
Wheat LAZY4 A Genome
SEQ ID NO 67:
MGIINWVQNRLNTKQEKKRSAAAAAAGASSVRNAPVRENSCRGQADDELPGDWSMLSIGTIGT
LGNEPTPAPAPDQAVPDFTIEEVKKLQDALNKLLRRAKSKSSSRGSTAGAGDEEQNLPLDRFL
NCPSSLEVDRRLSLRLQGADGGQNGEFSPDTQIILSKARELLVSTNGNGGGVKQKSFKFLLKN
MFACRGGFPPQPSLKDPVETKLEKLFKTMLQKKMSAPRQSNAASSSRKYYLEDKPMGRIQMDG
HHDEEEDDYGEDVFKWDKTDSDFIVLEV
Wheat LAZY4 A Genome
SEQ ID NO 68:
ATCATCAACTGGGTGCAGAATCGTCTGAACACCAAGCAGGAGAAGAAACGATCCGCCGCCGCC
GCCGCCGCGGGCGCGAGCTCGGTTCGCAATGCCCCGGTCCGGGAGAATAGTTGCCGCGGCCAG
GCCGACGACGAACTCCCCGGCGACTGGAGCATGCTCTCCATCGGAACCATCGGAACCCTCGGC
AACGAGCCCACGCCGGCGCCGGCGCCAGATCAGGCGGTGCCGGACTTCACCATCGAGGAGGTG
AAGAAGCTGCAGGACGCGCTGAACAAGCTACTCAGGCGCGCCAAGTCTAAGTCCAGCTCCCGC
GGCTCCACCGCCGGCGCCGGCGACGAGGAGCAGAACCTGCCGCTCGACAGGTTCCTCAACTGC
CCCTCCAGCCTCGAGGTCGACCGGCGGCTCTCGCTCAGGCTGCAGGGCGCCGATGGCGGGCAG
AACGGGGAGTTCTCGCCGGACACGCAGATCATACTCAGCAAGGCCAGGGAGCTCCTCGTCAGC
ACCAACGGCAACGGCGGGGGCGTCAAGCAGAAGTCCTTCAAGTTCCTCCTCAAGAACATGTTC
GCCTGCCGGGGCGGCTTCCCGCCGCAGCCCAGCCTCAAGGATCCAGTCGAAACAAAACTAGAG
AAGTTGTTTAAGACGATGCTTCAAAAGAAGATGAGCGCCCCGCGCCAGAGCAACGCGGCATCG
TCGTCGAGGAAGTATTACCTGGAGGACAAACCAATGGGAAGGATCCAAATGGATGGTCACCAC
GACGAGGAGGAGGATGACTACGGAGAAGATGTCTTCAAGTGGGACAAAACAGATTCAGATTTC
ATTGTTCTAGAGGTGTAA
Wheat LAZY4 D Genome
SEQ ID NO 69:
MGIINWWQNRLNTKQEKKRSAAAAAAGASSVRNAPVREKSCRGQADDELPGDWSMLSIGTLGN
EPTPAPAPAPDQAVPDFTIEEVKKLQDALNKLLRRAKSKSSSRGSTAGAGDEEQNLPLDRFLN
CPSSLEVDRRLSLRLQGADGGQNGEFSPDTQIILSKARELLVSTNGNGGGVKQKSFKFLLKNM
FACRGGFPPQPSLKDPVETKLEKLFKTMLQKKMSVPRPSNAASSSRKYYLEDKPMGRIQMDGR
HDEEEEEDYNDEDIFKWDKTDSDFIVLEV
Wheat LAZY4 D Genome
SEQ ID NO 70:
ATGGGGATCATCAACTGGGTGCAGAATCGCCTCAACACCAAGCAGGAGAAGAAACGATCCGCC
GCCGCCGCCGCCGCGGGCGCGAGCTCGGTTCGCAATGCCCCGGTCCGGGAGAAGAGCTGCCGC
GGCCAGGCCGACGACGAGCTCCCCGGAGACTGGAGCATGCTCTCCATCGGGACTCTCGGCAAC
GAGCCCACGCCGGCTCCGGCGCCGGCGCCAGATCAGGCGGTGCCGGACTTCACCATCGAGGAG
GTGAAGAAGCTGCAGGATGCGCTGAACAAGCTACTCCGGCGCGCCAAGTCCAAGTCCAGCTCC
CGCGGCTCCACCGCCGGCGCCGGCGACGAGGAGCAGAACCTGCCGCTCGACAGGTTCCTCAAC
TGCCCCTCCAGCCTCGAGGTCGACCGGCGGCTCTCGCTCAGGCTGCAGGGCGCCGACGGCGGG
CAGAACGGGGAGTTCTCGCCGGACACGCAGATCATACTCAGCAAGGCCAGGGAGCTCCTCGTC
AGCACCAACGGCAACGGCGGGGGCGTCAAGCAGAAGTCCTTCAAGTTCCTCCTCAAGAACATG
TTCGCCTGCCGGGGCGGCTTCCCGCCGCAGCCCAGCCTCAAGGATCCAGTGGAAACAAAACTG
GAGAAGTTGTTTAAGACGATGCTTCAAAAGAAGATGAGCGTCCCTCGCCCGAGCAACGCGGCA
TCGTCATCGAGGAAGTATTACCTAGAGGACAAACCAATGGGAAGGATCCAAATGGATGGTCGC
CACGACGAGGAGGAGGAAGAGGATTACAATGATGAAGATATCTTCAAGTGGGACAAAACAGAT
TCAGATTTCATTGTTCTAGAGGTGTAA
Wheat LAZY4 B Genome
SEQ ID NO 71:
MGIINWWQNRLNTKQEKKRSAAAAGASSVRNAPVREKSCRGQGDDELPGDWSMLSIGTLGNEP
TPAPAPDQGVPDFTIEEVKKLQDALNKLLRRAKSKSSSRGSTAGAGDEEQNLPLDRFLNCPSS
LEVDRRLSLRLQGADGGQNGEFSPDTQIILSKARELLVSTNGNGGGVKQNSFKFLLKNMFACR
GGFPPQPSLKDPVETKLEKLFKTMLQKKMSAPRQSNAASSSRKYYLEDKPMGRIQMDGRHDED
EEDDYGEDVFKWDKTDSDFIVLEV
Wheat LAZY4 B Genome
SEQ ID NO 72:
ATGGGGATCATCAACTGGGTGCAGAATCGGCTAAACACCAAGCAGGAGAAGAAACGATCCGCC
GCCGCCGCCGGGGCGAGCTCGGTTCGCAATGCCCCGGTCCGGGAGAAGAGCTGCCGCGGCCAG
GGCGACGACGAGCTCCCCGGCGACTGGAGCATGCTCTCCATCGGAACCCTCGGCAACGAACCC
ACGCCGGCGCCGGCGCCAGATCAGGGGGTGCCGGACTTCACCATCGAGGAGGTGAAGAAGCTG
CAGGACGCGCTGAACAAGCTACTCCGGCGCGCCAAGTCCAAGTCTAGCTCCCGCGGCTCCACC
GCCGGCGCCGGCGACGAGGAGCAGAACCTGCCGCTCGACAGGTTCCTCAACTGCCCCTCCAGC
CTCGAGGTCGACCGGCGGCTCTCGCTCAGGCTGCAGGGCGCCGATGGCGGGCAGAACGGGGAG
TTCTCGCCGGATACGCAGATCATACTCAGCAAGGCCAGGGAGCTCCTCGTCAGCACCAACGGC
AACGGCGGGGGTGTCAAGCAGAATTCCTTCAAGTTCCTTCTCAAGAACATGTTCGCCTGCCGG
GGCGGCTTCCCGCCGCAGCCCAGCCTCAAGGATCCAGTTGAAACAAAACTGGAGAAGTTGTTT
AAGACGATGCTTCAAAAGAAGATGAGCGCCCCGCGCCAGAGCAACGCGGCATCGTCGTCGAGG
AAGTATTACCTAGAGGATAAACCAATGGGGAGGATCCAAATGGATGGTCGCCACGACGAGGAT
GAGGAGGATGACTATGGAGAAGATGTCTTCAAGTGGGACAAAACAGATTCAGATTTCATTGTT
CTAGAGGTGTAG

1. A genetically altered plant wherein said plant comprises a dominant gain of function mutation in a LAZY4 nucleic acid sequence encoding for a protein having a LAZY4D motif wherein the LAZY4D motif is selected from SEQ ID NO. 3, 4, 5, 6 or 73.

2. The genetically altered plant of claim 1 wherein said plant comprises a mutation in a LAZY4 nucleic acid sequence encoding a mutant LAZY4 protein comprising a mutation in the LAZY4D motif (SEQ ID NO. 3, 4, 5, 6 or 73), optionally wherein one or more amino acid residue in the LAZY4D motif (SEQ ID NO. 3, 4, 5, 6 or 73) is substituted with another amino acid residue., and wherein optionally said amino acid residue that is substituted is selected from R, C, P, S, X, L, E, V, D, R, R wherein X is selected from S or C.

3. (canceled)

4. (canceled)

5. The genetically altered plant of claim 1 wherein the LAZY4 nucleic acid sequence comprises SEQ ID NO. 1 or a homolog, paralog, orthologue or functional variant thereof, and optionally wherein said homolog, paralog or orthologue is a LAZY4 nucleic acid sequence of a dicot or monocot plant, and optionally wherein said dicot or monocot plant is selected from rice (Oryza sativa), maize (Zea mays), wheat (Triticum aestivum), sorghum (Sorghum bicolor, Sorghum vulgare), Brassica, soybean, cotton and millet., and optionally wherein the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66 or a functional variant thereof.

6. (canceled)

7. (canceled)

8. (canceled)

9. The genetically altered plant of claim 1 wherein the mutation is in the endogenous LAZY4 nucleic acid sequence optionally wherein the mutation is introduced using targeted genome modification; and optionally wherein said mutation is introduced using a rare-cutting endonuclease, for example a TALEN, ZFN or CRISPR/Cas9.

10. (canceled)

11. (canceled)

12. The genetically altered plant of claim 1 wherein the plant has modulated root growth compared to a control plant.

13. (canceled)

14. (canceled)

15. A method for modulating root growth in a plant comprising introducing a dominant gain of function mutation into a LAZY4 nucleic acid encoding for a protein having a LAZY4D motif wherein the LAZY4D motif is selected from SEQ ID NO. 3, 4, 5, 6 or 73.

16. The method of claim 15 comprising introducing a mutation into a LAZY4 nucleic acid sequence encoding a LAZY4 protein wherein said mutant LAZY4 nucleic acid sequence encodes a mutant LAZY4 protein comprising a mutation in the LAZY4D motif, and optionally wherein one or more amino acid residue in the LAZY4D motif is substituted with another amino acid residue., and optionally wherein said amino acid residue that is substituted is selected from R, C, P, S, X, L, E, V, D, R, R wherein X is selected from S or C .

17. (canceled)

18. (canceled)

19. The method of claim 15 wherein the LAZY4 nucleic acid sequence comprises SEQ ID NO. 1 or a homolog, orthologue or functional variant thereto, optionally wherein said homolog or orthologue is a LAZY4 nucleic acid sequence of a dicot or monocot plant, and optionally wherein said dicot or monocot plant is selected from rice (Oryza sativa), maize (Zea mays), wheat (Triticum aestivum), sorghum (Sorghum bicolor, Sorghum vulgare), Brassica, soybean, cotton and millet, optionally wherein the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66, 68, 70, 72 or a functional variant thereof.

20. (canceled)

21. (canceled)

22. (canceled)

23. The method of claim 15 wherein said method comprises introducing the mutation into an endogenous LAZY4 nucleic acid sequence, optionally wherein the mutation is introduced using targeted genome modification, and optionally wherein said mutation is introduced using a rare-cutting endonuclease, for example a TALEN, ZFN or CRISPR/Cas9.

24. (canceled)

25. (canceled)

26. (canceled)

27. An isolated mutant LAZY4 nucleic acid sequence encoding a mutant LAZY4 protein comprising a dominant gain of function mutation.

28. The isolated mutant LAZY4 nucleic acid sequence of claim 27 wherein the mutant LAZY4 protein comprises a modification in the LAZY4D motif wherein the LAZY4D motif is selected from SEQ ID NO. 3, 4, 5, 6 or 73.

29. The isolated mutant LAZY4 nucleic acid sequence of claim 28 wherein the mutant LAZY4 protein comprises a substitution of one or more amino acid residue in the LAZY4D motif with another amino acid residue optionally wherein said amino acid residue that is substituted is selected from R, C, P, S, X, L, E, V, D, R, R wherein X is selected from S or C, optionally wherein the LAZY nucleic acid sequence comprises SEQ ID NO. 1 or a homolog, orthologue or functional variant thereof; and optionally wherein said homolog or orthologue is a LAZY4 nucleic acid sequence of a dicot or monocot plant; and optionally wherein said dicot or monocot plant is selected from rice (Oryza sativa), maize (Zea mays), wheat (Triticum aestivum), sorghum (Sorghum bicolor, Sorghum vulgare), Brassica, soybean, cotton and millet; and optionally wherein the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66, 68, 70, 72 or a functional variant thereof.

30. (canceled)

31. (canceled)

32. (canceled)

33. (canceled)

34. (canceled)

35. A vector or host cell comprising an isolated nucleic acid of claim 27.

36. (canceled)

37. A nucleic acid construct comprising a guide RNA that comprises a sequence selected from any of SEQ ID NOs. 45 to 60.

38. (canceled)

39. A method for producing a plant with modulated root growth, comprising introducing a dominant gain of function mutation into a LAZY4 nucleic acid having a LAZY4D motif wherein the LAZY4D motif is selected from SEQ ID NO. 3, 4, 5, 6 or 73.

40. The method of claim 39 comprising introducing a mutation into a LAZY4 nucleic acid sequence encoding a LAZY4 protein wherein said mutant LAZY4 nucleic acid sequence encodes a mutant LAZY4 protein comprising a mutation in the LAZY4D motif, and optionally wherein said mutation is introduced into the LAZY4 nucleic acid using targeted genome modification., and optionally wherein said mutation is introduced using a rare-cutting endonuclease, for example a TALEN, ZFN or CRISPR/Cas9; and optionally comprising introducing an endonuclease that targets a LAZY4 nucleic acid sequence into said plant.

41. (canceled)

42. (canceled)

43. The method of claim 42 comprising introducing an endonuclease that targets a LAZY4 nucleic acid sequence into said plant.

44. (canceled)

45. (canceled)

46. The method of claim 39 wherein one or more amino acid residue in the LAZY4D motif are substituted with another amino acid residue optionally wherein said amino acid residue that is substituted is selected from R, C, P, S, X, L, E, V, D, R, R wherein X is selected from S or C optionally wherein the LAZY4 nucleic acid sequence comprises SEQ ID NO 1 or a homolog, orthologue or functional variant thereto.

47. (canceled)

48. (canceled)

49. The method of claim 39 wherein said homolog or orthologue is a LAZY4 nucleic acid sequence of a dicot or monocot plant optionally wherein said dicot or monocot plant is selected from rice (Oryza sativa), maize (Zea mays), wheat (Triticum aestivum), sorghum (Sorghum bicolor, Sorghum vulgare), Brassica, soybean, cotton and millet, optionally wherein the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66, 68, 70, 72 or a functional variant thereof.

50. (canceled)

51. The method of claim 50 wherein the LAZY4 nucleic acid sequence is selected from SEQ ID NO. 8, 10, 12, 14, 16, 18, 20, 22, 24, 26, 28, 30, 32, 34, 36, 38, 40, 42, 44, 62, 64, 66, 68, 70, 72 or a functional variant thereof.

52. (canceled)

53. A method for identifying a plant with altered root growth compared to a control plant comprising detecting in a population of plants one or more polymorphisms in the LAZY4D motif of a LAZY4 nucleic acid sequence (SEQ ID NO. 1) wherein the control plant is homozygous for a LAZY4 nucleic acid that encodes a protein having a wild type LAZY4D motif (SEQ ID NO. 3, 4, 5, 6 or 73).

54. (canceled)


SEQUENCE LISTING (73 sequences)

SEQ ID NO: 1 - protein, 287 aa, Arabidopsis thaliana
MKFFGWMQNKLHGKQEITHRPSISSASSHHPREEFNDWPHGLLAIGTFGNKKQTPQTLDQEVIQEETVSNLHVEGRQAQDTDQELSSSDDLEEDFTPEEVGKLQKELTKLLTRRSKKRKSDVNRELANLPLDRFLNCPSSLEVDRRISNALCDEKEEDIERTISVILGRCKAISTESKNKTKKNKRDLSKTSVSHLLKKMFVCTEGFSPVPRPILRDTFQETRMEKLLRMMLHKKVNTQASSKQTSTKKYLQDKQQLSLKNEEEEGRSSNDGGKWVKTDSDFIVLEI

SEQ ID NO: 2 - DNA, 864 bp, Arabidopsis thaliana
atgaagtttttcgggtggatgcagaacaagctacatgggaaacaagagattactcatagaccaagcatatcctctgcttcttctcatcatccgagagaggagtttaacgattggcctcacggattactcgcgattggtacattcggtaacaaaaagcagacaccacaaacacttgatcaagaagtgattcaagaagagacagtgtctaacttacacgtggaaggtcgtcaagcacaagatacagatcaagagctttcttcctccgatgatctagaagaagatttcactcccgaagaagttgggaaactacagaaggagctgacgaaactcttgacgagaaggagtaagaaaaggaagtctgatgtgaatcgagaattagcgaatcttcctttggatagattcttgaattgtccttcgagtcttgaggtcgatagaagaatcagtaacgcgctttgtgatgagaaggaggaagacattgagcgtacaatcagtgttatcctagggagatgcaaagctatttctacagagagcaagaacaagacgaagaagaataaaagagatttgagcaaaacctctgtttctcatcttctcaagaagatgtttgtctgtacagaaggtttttctcccgttcctcgccctatcttgagagacacgtttcaagaaacaagaatggagaagttgctgagaatgatgctacacaagaaagttaacactcaagcttcatcaaagcaaacatcgacaaaaaaatacttgcaagacaagcaacagctctcgttgaagaacgaggaagaagaaggacgaagcagtaacgatggggggaaatgggtcaaaacagattctgatttcattgttcttgagatctga

SEQ ID NO: 3 - protein, 10 aa, Artificial Sequence
  LAZY4D motif X is any naturally occurring amino acid
  misc_feature (4)..(4): Xaa can be any naturally occurring amino acid
CPSXLEVDRR

SEQ ID NO: 4 - protein, 10 aa, Artificial Sequence
  LAZY4D motif
CPSSLEVDRR

SEQ ID NO: 5 - protein, 26 aa, Artificial Sequence
  LAZY4D motif
LANLPLDRFLNCPSSLEVDRRISNAL

SEQ ID NO: 6 - protein, 27 aa, Artificial Sequence
  LAZY4D motif X1 is ALWAYS PRESENT any naturally occurring amino acid X2 is either present or absent and if present, is any naturally occurring amino acid
  misc_feature (1)..(4): Xaa can be any naturally occurring amino acid
  misc_feature (16)..(16): Xaa can be any naturally occurring amino acid
  misc_feature (23)..(27): Xaa can be any naturally occurring amino acid
XXXXLPLDRFLNCPSXLEVDRRXXXXX

SEQ ID NO: 7 - protein, 295 aa, Arabidopsis thaliana
MKFFGWMQNKLNGDHNRTSTSSASSHHVKQEPREEFSDWPHALLAIGTFGTTSNSVSENESKNVHEEIEAEKKCTAQSEQEEEPSSSVNLEDFTPEEVGKLQKELMKLLSRTKKRKSDVNRELMKNLPLDRFLNCPSSLEVDRRISNALSAVVDSSEENKEEDMERTINVILGRCKEISIESKNNKKKRDISKNSVSYLFKKIFVCADGISTAPSPSLRDTLQESRMEKLLKMMLHKKINAQASSKPTSLTTKRYLQDKKQLSLKSEEEETSERRSSSDGYKWVKTDSDFIVLEI

SEQ ID NO: 8 - DNA, 888 bp, Arabidopsis thaliana
atgaagttcttcgggtggatgcagaacaagcttaatggggatcataacagaacaagcacttcctctgcttcttctcatcatgtgaagcaagaaccaagagaggagtttagcgactggcctcacgcgctgcttgctattggaacattcggtacaacaagcaatagtgtgagcgaaaacgagagcaagaatgttcatgaagagattgaagcggagaagaagtgtacggcacaatccgagcaagaagaagagccttcttcctctgtcaatcttgaggatttcactcctgaagaggttggaaagttgcagaaagagttgatgaagctcttgtcaagaactaagaaaaggaagtctgatgtgaatagagagctcatgaaaaatcttcctttagatagattcttgaactgtccatcgagtttagaggtggataggcgaatcagcaatgcgcttagcgctgttgtggattcgtcagaggagaataaggaggaagatatggagcgaacgattaacgttattctaggtagatgcaaagagatatcaatagagagtaagaataacaagaagaagagagacataagcaagaactctgtctcatatcttttcaagaagatttttgtctgcgcagatgggatttctacagccccaagccctagcttgagagacacgcttcaagaatcaagaatggagaagttgttgaagatgatgctccataagaagattaatgctcaagcctcctcgaaaccaacatcattgacaacaaagagatacttgcaagacaagaaacagctctcactgaagagtgaggaagaagaaactagcgaaagaagaagtagtagcgatggatataaatgggtcaaaacagattctgatttcatagttctcgagatatga

SEQ ID NO: 9 - protein, 288 aa, Zea mays
MQDRFNGKHDKRRPEAINSGSARESCRQDDRAREGKSRNDGGDWPAPQHGLLSIGTLGDDDPPPPRASSQADDVLDFTIEEVKKLQDALNKLLRRAKSKSSSSSSSSRGSGASATDEDRRASHSQLPLDRFLNCPSSLEVDRRVSLIRHDGGGESGEFSPDTQIILSKARDLLVHSNGTAIRKKSFKFLLKKMFVCHGGFAPAPSLKDPVESRMEKLFRTMLQKKMNARPSNAAVSSRKYYLDDKPSGRMMTRDGRRRHDGEDDDEKGSDRIKWDKTDTDCKNIFIRC

SEQ ID NO: 10 - DNA, 867 bp, Zea mays
atgcaggatcgcttcaacggtaaacacgataagaggcgacccgaggccattaactcgggatcagctcgcgaaagctgccgccaagacgaccgcgcgcgcgagggcaagagccgcaacgacggcggcgactggccggcgccacagcacggcctcctgtcgatcgggacgctgggagacgacgacccgccgccgccgcgcgcgtcgtcgcaggccgacgacgtgctggacttcaccatcgaggaggtgaaaaagctccaggacgcgctgaacaagctgctccggcgcgccaagtccaagtccagctccagctccagctcctcccgcgggtcgggcgccagcgccaccgacgaggaccgccgcgccagccacagccagctgccgctcgacaggttcctcaactgcccctccagcctcgaggtcgaccggagggtctcgctgatcaggcacgacggtggtggcgagagcggcgagttctcgccggacacgcagatcatactcagcaaggccagggatctcctcgtccacagcaacggcaccgccatcaggaagaagtcgttcaagttcctcctgaagaagatgttcgtctgccatggcggcttcgcccccgcgccgagcttgaaggatccagttgaatcgagaatggagaagttgttcagaacgatgcttcagaagaagatgaatgctcgcccgagcaacgctgcagtgtcatccaggaagtactacctcgacgacaagccgagcgggaggatgatgacacgggatggtcgtcgtcgtcacgatggagaggacgatgacgagaagggctctgacagaatcaagtgggataaaactgatactgactgtaagaacatatttatacgctgctag

SEQ ID NO: 11 - protein, 263 aa, Glycine max
MKFLSWMQNKLGGKQDNRKPNTHTTNTTTYLAKQEPREEFSDWPHGLLAIGTFGNKSEIKEDLDDQNTQEDPSSSEEIADFTPEEIGNLQKELTKLLRRKPNVEKEISELPLDRFLNCPSSLEVDRRISNALCSESEDKEEDIEKTLSVIIDKCKDICADKRKKAIGKKSISFLLKKIFVCRSGFAPTPSLRDTLQESRMEKLLRTMLHKKIYTQNSSRSPLVKKGIEDKKMTRKRNEDESDERNGDGCKWVKTDSEYIVLEI

SEQ ID NO: 12 - DNA, 846 bp, Glycine max
atgcactctaagctcattcatccccccctatcttttagccttagtccttccacaatgaagttcctcagctggatgcaaaataaacttggtggaaaacaagacaacagaaaaccaaatacacatactactaatactactacatatcttgcaaaacaagagcctagagaagaattcagcgattggcctcatggtttactagcaattggaacatttggaaataagagtgaaatcaaagaagacttagacgaccaaaatacacaagaggatccatcttcatcagaggaaatagcagacttcactcctgaagaaattgggaatctacagaaggagttaactaaactcctgagacgaaaacccaatgtggaaaaggaaatttctgagctccctctggacagatttcttaactgcccttcaagcttggaggttgataggagaatcagtaatgcactatgcagtgaatcagaagataaggaagaagatattgagaagacactgagtgtgataattgataaatgcaaagacatttgtgcagataaaagaaagaaagcaattgggaagaaatccatttctttccttctgaagaagatatttgtttgtagaagtggatttgctccaacacctagcctaagagatacccttcaagagtcaagaatggagaagcttttgaggacaatgcttcacaagaaaatttacacccaaaactcttctcggtcaccgttggtgaagaagggcatagaggataagaagatgacaaggaagaggaatgaggatgaatcagatgagagaaatggtgatggctgtaaatgggtcaagactgattctgaatatattgttctagagatataa

SEQ ID NO: 13 - protein, 284 aa, Glycine max
MHSKLVHPPLSFSLSPSTMKFLSWMQNKLGGKQDNRKPNAHTTTTTTTTTYHPKQEPREEFSDWPHGLLAIGTFGNKTAIKEDLDDQNTQEDPSSSEEIADFTPEEIGNLQKELTKLLRRKPNVEKEISELPLDRFLNCPSSLEVDRRISNALCSESEDKEEDIEKTLSVIIDKCKDICADKRKKAMGKKSISFLLKKIFLCRSGFAPTPSLRDTLQESRMEKVLRTMLHKKICTQNSSRSPLVKKCIEDKKMTRKKNEDESDERNGDGCKWVKTDSEYIVLEI

SEQ ID NO: 14 - DNA, 855 bp, Glycine max
atgcactctaagctcgttcatccccccctatcttttagccttagtccttccacaatgaagttcctcagctggatgcaaaataaacttggtggaaaacaagacaacagaaaaccaaatgcacatactacaacaactactactactactacatatcatccaaaacaagagcctagggaagaattcagcgattggcctcatggtttactagcgattggaacatttggaaacaagactgcaatcaaagaagacttggatgaccaaaatacacaagaggatccatcttcttcagaggaaatagcagacttcactcctgaagaaattgggaatctacagaaggagttaactaaacttctgagacgaaaacccaatgtggaaaaggagatttctgagcttcctctggacagatttcttaactgtccttcaagcttggaggttgataggagaatcagtaatgcactatgcagtgaatcagaagataaggaagaagatattgagaaaacactaagtgtaataattgataaatgcaaagacatttgtgcagataaaagaaagaaagcaatggggaagaaatctatttctttccttctgaagaagatctttctttgtagaagtggatttgctccaacaccaagccttagagatacccttcaagagtcaagaatggagaaggttttgaggacaatgctccacaagaaaatttgcacccaaaattcttctcggtcaccgttggtgaagaagtgcatagaggacaaaaagatgacaaggaagaaaaatgaggatgaatcagatgagagaaatggtgatggctgtaaatgggtcaagactgattctgaatatattgttctagagatataa

SEQ ID NO: 15 - protein, 282 aa, Glycine max
MGFTFPLILQLEVVDIGKFFGTQKARLYGSKGLRNWRGEADDAKQEPREEFSDWPDGLLAIGTFGNSNEVKEKTEKHILREDPSSSEEIADFTPEEIGKLQKELTKLLRQKPNVEKEIAELPLDRFLNCPSSLEVDRRISNVLCSDSEDKDKDEEEREKEEEEDIEKTLSVILGKFKEICANNSKKAIGKKSISFLLKKMFVCRSGFAPAPSLKDTLQLQESRMEKLLRIILHKKINSQHSSRALSLKKRLEDRKMPKEDEAENDDGCKWVKTDSEYIVLEI

SEQ ID NO: 16 - DNA, 807 bp, Glycine max
atgaagttcctcagctggatgcaaaacaaaattggtggaaaacaagataacagaaaaccaaacacatatacaactactcatgatgcaaagcaagagcctcgtgaagaattcagcgattggcctgatggtttactagccattggtacatttggaaatagcaatgaagtaaaagaaaagacagagaagcacattctcagagaggatccatcctcgtcagaggaaatagcagacttcactcctgaagaaatcgggaaactacaaaaagagttaactaaactgttgagacaaaaacccaatgtggaaaaggaaattgctgagcttcctctggacagatttctcaattgtccatcaagcttggaggttgataggagaatcagtaatgtactttgcagtgattcagaagacaaagataaagatgaagaagaaagagaaaaagaagaagaagaagatattgaaaagacacttagtgtcatacttggtaaattcaaagagatttgtgcaaataacagcaagaaagcaattgggaagaaatcaatttcatttttgctgaagaagatgtttgtttgtagaagtggatttgctccagcaccgagccttaaagacacccttcagctccaagaatcaagaatggagaagcttttaaggataattcttcacaagaaaataaactcccaacattcttctcgggcattgtccctcaagaagcgcctcgaggacaggaagatgccaaaggaggatgaagctgaaaatgatgatggctgtaaatgggtcaagactgattctgaatatattgttttagagatttaa

SEQ ID NO: 17 - protein, 289 aa, Brassica rapa
MKLFGWMQNKLHGKQGNTHRPSTSSASSHQPREEFSDWPHGLLAIGTFGSVTKEQIPIETVQEEKPSNLHVEGQAQDRDQDLSSSGDLEDFTPEEVGKLQKELTKLLTRKNKKRQSDVNRELANLPLDRFLNCPSSLEVDRRISNALSGGCGDCDENEEDIERTISVILGRCKAISTESNSKKKKTKKDLSKTSVSYLLKKMFVCTEGFSPLPKPSVRDTFQESRMEKLLRVMLLKKINAQAPSKETPTNRYVQDKQQLSLKNEEEEGSSSSDGCKWVKTDSDFIVLEI

SEQ ID NO: 18 - DNA, 870 bp, Brassica rapa
atgaagctctttggatggatgcagaacaagctacatgggaaacaagggaacactcatagaccaagcacatcctctgcttcttctcatcaaccacgagaggagttcagcgactggcctcatggattacttgcgattggaacgttcggtagtgtgactaaagagcaaataccaatagagactgttcaagaagagaagccctctaacttgcacgtggaaggtcaagcgcaagatagagatcaagatctttcctcctccggtgatttagaagatttcactccagaggaagttgggaaactgcaaaaggagctgacgaagctcttgacaagaaagaacaagaagagacagtctgatgtgaacagagaacttgcgaatcttcctctggatagattcttgaattgtccttcgagtcttgaagtcgatagacgaatcagcaacgctctttctggtggttgtggagattgtgatgagaacgaagaagacattgagcgtacaatcagtgttatcttgggaagatgcaaagccatttctacagagagtaacagtaagaagaagaagactaagaaagatttgagcaaaacctctgtctcttatctcctcaagaagatgtttgtctgtacagaagggttctctcctcttcctaaacctagcgtgagagacacgtttcaagaatcaagaatggaaaagttactgagggtgatgctactcaagaagattaatgctcaagctccctcgaaggaaacaccaacgaatagatacgtgcaagacaagcaacagctttcattaaagaatgaggaagaagaaggaagtagtagtagcgatgggtgtaaatgggtcaaaacagattctgatttcattgttcttgagatctga

SEQ ID NO: 19 - protein, 291 aa, Brassica rapa
MKFFGWMQNKLHGKQGNTHRPSISSASSHQPREEFSDWPQGLLAIGTFGSVAKEQTQIQVVQEVIQEENPSNVHVEGQVQDEDQDLSFSGDLEDFTPEEVGKLQKELTKLLTRKTKKRKSDVNRELANLPLDRFLNCPSSLEVDRRISNAISSGGYSNENEEDIERTISVILGRCKAISTESSNKKKKSKRDMSKTSVSYLLKKMFVCSGGFSPLPNPSLRDTFQESRMEKLLRVMLHKKINAQAPSKETSTKRYVEDKQQLALKNEEEEGRSSDGSKWVKTDSDFIVLEI

SEQ ID NO: 20 - DNA, 978 bp, Brassica rapa
atgcagaacaagctacatgggaaacaagggaacactcatagaccaagcatatcttctgcttcttctcatcaaccaagagaggagttcagcgactggcctcaaggattacttgcgattggaactttcggtagtgtggccaaagagcaaacacaaatacaagttgttcaagaagtgattcaagaggagaatccctctaacgtgcacgtggaaggtcaagttcaagatgaagatcaggatctttctttctccggtgatcttgaagattttactcccgaggaagttgggaaactgcaaaaggaactgacgaagctcttgacaagaaagaccaagaaaaggaagtcagatgtgaacagagaacttgcgaatcttcccctggatagattcttgaattgtccttcgagtcttgaagtcgacagacgaatcagcaacgcgatttctagtggtggatattctaacgagaacgaagaagacattgaacgtaccatcagtgttatcttgggaagatgcaaagctatttctacagagagtagcaataaaaagaagaagagtaagagagatatgagcaaaacctctgtttcttatcttctcaagaagatgtttgtttgttcaggagggttctctcctcttcctaaccctagcttgagagacacgtttcaagaatctagaatggaaaagttactgagggtgatgctacacaagaagattaatgctcaagctccctcgaaggaaacatcaacaaaaagatacgtggaagataagcaacagcttgcactaaagaacgaggaagaagaaggaagaagtagtgatgggagcaaatgggttaaaacagattctgattgtgagtttcagatcttttggtttcttaaatttttttttgaaaaaaatgttcaagaattgattagatcttcttctttgttttggttgcagtcattgttcttgagatctgatcccattttccattcttcatgttacaggtaa

SEQ ID NO: 21 - protein, 291 aa, Brassica rapa
MKLFGWMHNKLHGKQANTHRPRTSSACSHQSREEFSDWPHGLLAIGTFGTLIKDQTPIHVVQEVIQEEKTSNMHVEGKAQDRNHDLSLSDDLEDFTPEEVGKLQNELTKLLTRKNKKRKSDVNKELENLPLDRFLNCPSSFEVDRRISNAFSGGGDSDENQEDIERAISTILGRCKAISTGSKSKMKAKRDWSKTSVSYLLKKMFVCTEGHSPLPNPGLRDTFQESRMEKFLRVMLLKKINTRACPKETSTCRYVQDRQQLSLKNKEEEGRSSSDGSTWVKTDSDFIVLEI

SEQ ID NO: 22 - DNA, 996 bp, Brassica rapa
atgcataataagctacatggtaaacaagcgaatactcatagaccaagaacatcatctgcttgttctcatcaatcacgagaagagttcagtgattggcctcacggattacttgccattggaacgttcggtaccttgatcaaagatcaaaccccaatacatgttgttcaagaagtgattcaagaagagaagacttctaacatgcacgtggaaggtaaagcgcaagatagaaatcacgatctttctttatccgatgatcttgaagattttactcccgaggaagttgggaaactacaaaatgagctgacgaagctcttgacaagaaagaacaagaagaggaagtctgatgtgaacaaagaacttgagaatcttcctttggatagattcttgaattgtccttcgagttttgaagtcgatagacgaatcagcaacgcgttttcaggtggtggagattctgatgagaaccaagaagacattgagcgtgcgattagtactattttggggagatgcaaagctatttctacagggagtaaaagtaagatgaaggctaagagagattggagcaaaacctctgtttcttatctcctcaagaagatgtttgtatgtacagaggggcactctcctcttcctaaccctggcttgagagacacgtttcaagaatcgagaatggagaagtttctgagagtaatgctactcaagaagattaatactcgagcttgtccaaaggaaacatcaacgtgtagatacgtgcaagacaggcaacaactttcattaaagaataaggaagaagaaggaagaagtagtagcgatgggagtacatgggtcaaaacagattctgactgtgagtttaaaatctttttatttcttttcaaaacaaaagaagtcgtccatgaactaattctattttcatcatcttctttttggttgcagtcattgttcttgagatctgattcactttacccctactcagattcttacaggaaagtacaggtaatatag

SEQ ID NO: 23 - protein, 285 aa, Hordeum vulgare
MGIINWVQNRLNTKQEKKRSAAGAAAASSARNAPDWEKSCRGQADDELPGDWSMLSIGTLGNEPTPAPAPDQAVPDFTIEEVKKLQDALNKLLRRAKSKSSSRGSTAGAGDEEQNLPLDRFLNCPSSLEVDRRLSLRLQAADGGQNGEFSPDTQIILSKARELLVSTNGNGGGVKQKSFKFLLKNMFACRGGFPPQPSLKDPVETKLEKLFKTMLQKKMSVPRPSNAASSSRKYYLEDKPMGRIHMDGSHEEEEDYNVEDIFKWDKTDSDCKSLELINFTAALTN

SEQ ID NO: 24 - DNA, 858 bp, Hordeum vulgare
atggggatcatcaactgggtgcagaaccgcctcaacaccaagcaggagaagaaacgatcggccgccggcgccgctgccgccagctcggctcgcaatgccccggactgggagaagagttgccgcggccaggccgacgacgagctccccggcgactggagcatgctctccatcggaaccctcggcaacgagcccacgccggcgccggcgccagatcaggctgtgccggacttcaccatcgaggaggtgaagaagctgcaggacgcgctgaacaagctactccggcgcgccaagtccaagtccagctcccgcggctccaccgccggcgccggcgacgaggaacagaacctgccgctcgacaggttcctcaactgcccctccagcctcgaggtcgaccggcggctctcgctcaggctgcaagccgccgacgggggacagaacggggagttctcgcctgacacgcagatcatactcagcaaggccagggagctcctcgtcagcaccaacggcaatggcgggggcgtcaagcagaagtccttcaagttcctcctcaagaacatgttcgcctgccggggcggcttcccgccgcagcccagcctcaaggatccagttgaaacaaaattggagaagttgtttaagacgatgcttcaaaagaagatgagcgtccctcgcccgagcaacgcggcatcgtcgtcgaggaagtattacctagaggataaaccaatggggaggatccacatggatggtagccacgaggaggaggaggattacaatgttgaagatatcttcaagtgggacaaaaccgattcagattgtaagtcgctagagttgataaatttcactgctgccttaacaaattaa

SEQ ID NO: 25 - protein, 295 aa, Oryza sativa
MGIINWMQNRLSTAKQDKRRTEAAAVASSARRRGGGGGESCRQEEARDEIKIAGDHLLSIGTLGNESPPRPPAAAAATAAEEVADFTIEEVKKLQEALNKLLRRAKSTKSGSRRGSTAAEHDADERSSSSSSSGSQLLLPLDRFLNCPSSLEVDRRVAAADGEFSPDTQIILSKARDLLVNTNGGGAIKQKSFRFLLKKMFVCRGGFSPSPAPPPTLKDPVESRIEKLFRTMLHKRMNARPSNAAASSSRKYYLEDKPREKMQREHLHDDEDDDENAEDIFKWDKTDSDFIVLEM

SEQ ID NO: 26 - DNA, 888 bp, Oryza sativa
atggggattattaactggatgcagaatcgactcagtactgctaaacaagacaagagacgaactgaagctgctgctgtggcctcgtcagctcgcagacgaggaggagggggaggagagagttgccgccaagaagaagctcgcgacgagatcaagatcgccggagatcacctcctctccatcggcacgctcgggaacgagtcgccgccgcgaccgccggcggcggcggcggcgacggcggcagaggaggtggcggacttcaccatcgaggaggtgaagaagctgcaggaggcgctgaacaagctgctccggcgagccaagtccaccaagtccggcagccgccgcggctcgacggcggcggagcacgacgccgacgagcgctcctcctcctcctcctcctccggcagccagctgctgctgccgctcgacaggttcctcaactgcccctccagcctcgaggtcgaccggcgcgtggcggcggccgacggcgagttctcgccggacacgcagatcatcctcagcaaggcgcgcgacctcctcgtcaacaccaatggcggcggcgccatcaagcagaaatccttcaggttcctcctcaagaagatgttcgtctgccgcggcggcttctcgccgtcgccggcgccgccgcccaccttgaaggatccagtcgaatcaagaatcgaaaagttgttcaggacgatgcttcacaagaggatgaacgctcgaccgagtaatgctgcggcgtcgtcgtcgaggaaatactatcttgaggataagccgagggagaagatgcaaagggagcatctccatgatgatgaagatgatgatgagaatgcagaagatatctttaaatgggacaaaactgattcagatttcattgttctggagatgtag

SEQ ID NO: 27 - protein, 323 aa, Oryza sativa
MGIINWMQNRLSTAKQDKRRTEAAAVASSARRRGGGGGESCRQEEARDEIKIAGDHLLSIGTLGNESPPRPPPAAAATAAEEVADFTIEEVKKLQEALNKLLRRAKSTKSGSRRGSTAAEHDADERSSSSSSSGGQLLLPLDRFLNCPSSLEVDRRVAAADGEFSPDTQIILSKARDLLVNTNGGGAIKQKSFRFLLKKMFVCRGGFSPSPAPPPTLKDPVESRIEKLFRTMLHKRMNARPSNAAASSSRKYYLEDKPGEKMQREHLHDDEDDDENAEDIFKWDKTDSDCNHCSGDVDRDARFNAIIIVCTMISDTVGVRFTI

SEQ ID NO: 28 - DNA, 972 bp, Oryza sativa
atggggattattaactggatgcagaatcgactcagtactgctaaacaagacaagagacgaactgaagctgctgctgtggcctcgtcagctcgcagacgaggaggagggggaggagagagttgccgccaagaagaagctcgcgacgagatcaagatcgccggagatcacctcctctccatcggcacgctcgggaacgagtcgccgccgcgaccgccgccggcggcggcggcgacggcggcagaggaggtggcggacttcaccatcgaggaggtgaagaagctgcaggaggcgctgaacaagctgctccggcgagccaagtccaccaagtccggcagccgccgcggctcgacggcggcggagcacgacgccgacgagcgctcctcctcctcctcctcctccggcggccagctgctgctgccgctcgacaggttcctcaactgcccctccagcctcgaggtcgaccggcgcgtggcggcggccgacggcgagttctcgccggacacgcagatcatcctcagcaaggcgcgcgacctcctcgtcaacaccaatggcggcggcgccatcaagcagaaatccttcaggttcctcctcaagaagatgttcgtctgccgcggcggcttctcgccgtcgccggcgccgccgcccaccttgaaggatccagtcgaatcaagaatcgaaaagttgttcaggacgatgcttcacaagaggatgaacgctcgaccgagtaatgctgcggcgtcgtcgtcgaggaaatactatcttgaggataagccgggggagaagatgcaaagggagcatctccatgatgatgaagatgatgatgagaatgcagaagatatctttaaatgggacaaaactgattcagattgtaatcattgttctggagatgtagaccgagacgcacgattcaatgcgatcattattgtttgcacaatgatttcagatacagttggtgtacgtttcaccatatag

SEQ ID NO: 29 - protein, 308 aa, Oryza sativa
MGIVSWVQGRLGGRTSAAAESRGLAAGNGNPSLVAAVVAPGKERKHQQVVPDDLAGDQWPTPATHLFSIGTLGNDELPEQGEEEEDLPEFSVEEVRKLQDALARLLLRARSKNYSEAVATAAATATCCGGGGADSGLPLDMFLNCPSSLEVDRRAQRDHGGGGAAVGLSPGTKMILTKAKDILVDGNTRNTTTSGGDIKNKSFKFLLKKMFVCHGGFAPAPSLKDPTESSMEKFLRTVLGKKIAARPSNSPASRTYFLEGNNAHGDDHRLCRRRRPRCGEEEEEEEENKGEESCKWDRTDSEYIVLEI

SEQ ID NO: 30 - DNA, 927 bp, Oryza sativa
atggggatcgtcagctgggtgcaggggaggctgggtgggaggacgtcggcggcggcggagagcagagggctcgccgccggcaacggcaatccttcgctggtcgcggcggtcgttgcgccaggcaaggagaggaagcatcagcaggttgttcctgacgatctcgccggcgatcaatggccgactccggcgactcatctcttctccatcggcacgttgggcaacgacgagttgccggagcagggggaggaggaggaggacctgccggagttcagcgtcgaggaggtgaggaagctccaggacgcgctggcgaggctcctcctgcgcgccaggtccaagaattattccgaggccgtcgccaccgccgccgccaccgccacctgctgcggcggcggcggcgcggacagtggcctgccgctcgacatgttcctcaactgcccttccagcctcgaggtggacaggagagcacagcgcgatcacggcggcggaggcgccgccgtcggcctctcgccgggcaccaagatgatactcaccaaggccaaggacattctcgtcgacggcaacaccagaaacaccaccaccagcggcggcgacatcaagaacaagtcattcaagttccttctcaagaagatgttcgtctgccatggcggcttcgcgccggctccgagcttgaaggacccgacggaatcatcaatggagaagtttctccgaacggtgctcggcaagaagatcgctgcccggccgagcaattcaccggcgtcgaggacatacttcttggagggtaacaatgcacatggtgatgaccatcgcctttgtcgccgccgtcgtcctcgttgcggcgaagaagaagaagaggaggaggagaacaagggggaagaaagttgtaaatgggacaggacagattctgaatatattgttcttgagatatga

SEQ ID NO: 31 - protein, 290 aa, Sorghum bicolor
MGIINWMQNRFNGKHEKRRPEATAAAAAAAFSSAHESCRQDHGREDKIPTGDWPPQGLLSIGTLGDDPPPAAGDGGGGPPRASQADVLDFTIEEVKKLQDALNKLLRRAKSKSSSSRGSGATDEDRASQLPLDRFLNCPSSLEVDRRISLRHAAGDGGGENGEFSPDTQIILSKARDLLVNSNGTTIKKKSFKFLLKKMFVCHGGFAPAPSLKDPVESRIEKLFRTMLQKKMNNARPSNAAVSSRKYYLEDKPSGRMMIRDGHHDEEDDEKGSDRIKWDKTDTDFIVLEI

SEQ ID NO: 32 - DNA, 873 bp, Sorghum bicolor
atggggatcattaactggatgcagaatcgcttcaatggtaaacatgagaagaggcgacccgaggccaccgccgccgccgccgccgccgcctttagctcagctcacgaaagctgccgccaagaccacggtcgcgaggacaagatccccaccggcgactggccgccacagggcctcctctcgatcgggacactgggcgacgacccaccaccggcggcgggagatggaggtggaggcccgccgcgcgcgtcgcaggccgatgtgctggacttcaccatcgaggaggtgaagaagctgcaggacgcgctgaacaagctgctccggcgcgccaagtccaagtccagctcctcccgcgggtcgggcgccaccgacgaggaccgcgctagccagctgccgctcgacaggttcctcaactgcccatccagcctcgaggtcgaccggaggatctccctgaggcacgccgccggcgacggtggtggcgagaatggcgagttctcgccagacacgcagatcatactcagcaaggccagggatctcctcgttaacagtaacggcaccaccatcaagaagaagtcgttcaagttcctcctcaagaagatgttcgtctgccatggcggcttcgcccccgcaccgagcttgaaggatccagttgaatcaaggatagagaagttgttcagaacgatgcttcagaagaagatgaacaatgctcgcccgagcaatgctgcagtgtcatccaggaagtactacctcgaagacaaaccgagtgggaggatgatgatacgggatgggcatcacgatgaagaggatgatgaaaagggttctgacagaatcaagtgggataaaactgatactgacttcattgttctggagatctaa

SEQ ID NO: 33 - protein, 282 aa, Sorghum bicolor
MGIINWMQNRFHGKTENRIFDGGATATSSYRGAGAQERQETIIREPEKHLDAEPWPQAPAGLLSIGTLGSEEPPPPAAQDLPEFTVEEVKKLQDALAMLLRRAKSKSSARGSAAGEDRPPLDRFLNCPSCLEVDRRVQTTAKHGECGGGQEGEGDLSPDTKIILTRARDLLDSGGGIKQRSFKFLLKKMFACNGGFSAAPPRSLKDPVESRMEKFFRTVIGKKMNASSGNRSSTSRKYFLEDGTSKGKRRGARRCGCQEEEEEREESCKWDRTDSEFIVLEI

SEQ ID NO: 34 - DNA, 849 bp, Sorghum bicolor
atggggatcatcaactggatgcagaacagattccatgggaagaccgagaacagaatctttgacggcggcgcaactgccaccagttcatatagaggcgctggagcccaagagagacaagagacgatcattcgtgaaccagagaagcatctcgacgccgagccatggcctcaggcgccggcggggctcctctccatcggcacgctcggcagcgaggagcctccgccgccggcagcgcaggacctgccggagttcaccgtggaggaggtgaagaagctccaggacgcgctggccatgctcctgcggcgcgccaagtccaagtccagcgcccgcggctccgcggccggcgaggacaggccgccgctggacaggttcctcaactgcccgtcctgcctggaggtggacaggcgggtccagacgacggccaagcacggcgagtgcggcggtggccaggaaggcgaaggagacctctcgccggacaccaagatcatactgaccagggccagagacctgctcgacagcggcggcggcatcaagcagaggtcgttcaagttcctgctcaagaagatgttcgcctgcaatggcggcttctcggcggcgccgcctcggagcttgaaggacccagtggagtcaagaatggagaagttcttccgaacggtgatcgggaagaagatgaatgccagctcgggcaacaggtcgtcaacgtcgaggaagtacttcttggaggatggaaccagcaaggggaagaggcgaggtgctcgtcgttgtggttgccaagaggaggaggaggagagggaagagagctgcaaatgggacagaacagattctgaattcattgttttggagatatga

SEQ ID NO: 35 - protein, 279 aa, Gossypium raimondii
MKFFGWVQNKLNGKPGRSKPQTDSATNYMKQEPRQEFSDWPHGLLAIGTFGNNNDMIENPPSQNTARQDPFDIREEHEPSSSEDLHEFTPEEVGKLEKELTKLLSRKPASDVKKELANLPLDRFLNCPSSLEVDRRISNAVCSDSGDKSDQEDIDRTISVILGRCKDICAEKNKKSIGKKSLSFLLKKMFACGSGFSPAPSLRDVLQESKMERLLRVMLHKKIYNQNPSGASAVKKYLEDRQSPKRRNKLNNEDETQERKSEDGYKWVKTDSEYIVLEI

SEQ ID NO: 36 - DNA, 840 bp, Gossypium raimondii
atgaaattctttggttgggtccaaaataagcttaatgggaaaccggggcgcagtaaaccacaaacagattctgctactaattacatgaaacaggagcctcgacaagagttcagcgattggcctcatggattgttggctataggaacgtttggcaacaataatgacatgatagaaaatcctccatcccaaaacaccgcccgacaagatccgtttgatattcgcgaggaacacgagccgtcctcatcggaggatttacacgaatttacgcccgaagaagtcgggaaactagaaaaggaattaaccaaactcttgtcccgaaaaccggcttccgatgttaaaaaggaactagcaaatctaccattggataggtttcttaactgtccatcgagcttggaagttgataggaggattagcaatgcggtttgtagtgattcaggggataaatcagatcaagaagacattgatcgaaccattagtgttattctcggccgatgcaaagacatttgcgctgaaaaaaacaagaaatccatcggcaaaaaatcgctttctttccttttgaagaagatgtttgcttgcggcagtggattttcacctgccccgagcttgagagatgtgctgcaagaatcgaaaatggagaggcttttgagggtaatgcttcacaagaagatttacaatcagaacccttctggagcatcagctgtgaagaaatatttagaagacagacagtctccgaaaaggcgaaataaattaaataatgaagatgaaacccaggagaggaagagtgaagatggatataaatgggtgaagacagattctgaatatattgttctggagatctaa

SEQ ID NO: 37 - protein, 276 aa, Gossypium raimondii
MKFFGWMQNKLNGKQGPSKSNTISATYHMKQEPREEFSDWPHGLLAIGTFGNNELKENPESQSTIQQEPIEIQDQEPCSSDDLQEFTVEEVGKLQKELTKLLSRKPNPNTKKEVASLPLDRFLNCPSSLEVDRRFSNAVCSDAGERSEEDIDRTISIILGRCKDIRGEDNKKKAIGKKSISFLLKKMFVCSGGFPPTPTLRDTLQESRMEKLLRVMLHKKIYSQNPTREPSMKKYLEDKQTPKRQKIPDENETVERKSEDGGKWVKTDSEYIVLEI

SEQ ID NO: 38 - DNA, 831 bp, Gossypium raimondii
atgaagttctttggttggatgcaaaataagcttaatgggaaacaaggacccagcaagtcaaatacaatatctgctacttatcatatgaaacaagagcctcgggaggagttcagtgattggccacatggactgttagcaatagggacatttggtaacaatgagcttaaagaaaaccctgaatcccaaagcaccattcaacaggaacccattgagattcaagaccaagagccatgttcgtccgatgatttacaggagttcacggtcgaagaagtcgggaaactacaaaaggaactaacgaaactcttgtcccgaaaaccgaaccccaacacaaaaaaagaagtagcaagtttaccattggatagatttcttaattgtccatcaagcttggaagtggatagaaggtttagcaatgcggtttgcagtgatgcaggggagagatcggaggaagacatcgatcgaaccattagcattatcctcggcagatgcaaagacatacgtggtgaagataataagaaaaaggccattgggaagaaatcaatttctttccttttgaagaagatgtttgtttgttcaggtggatttccacctacaccaactttgagagatacactacaagaatcaagaatggagaagcttttgagggtaatgcttcacaagaagatttacagtcaaaatccaactagagaaccatcaatgaagaaatacttggaggacaagcaaacacccaaaaggcaaaaaattccagatgaaaatgaaacagtggagagaaagagtgaagatggaggtaaatgggtgaaaacagattctgaatatattgttctagagatataa

SEQ ID NO: 39 - protein, 279 aa, Nicotiana attenuata
LQFFSWMQNKFNGGQGNRSMPNEVQTKKRPRNEEFNGWPDSLLAIGTFGTSSSNLKAKSESQNVQNQERDEIILDDNINEQSSSPDLAEFTPEEVGKLQKELTKLLSKKPAAKLIDQGRQDGDLPLDRFLNCPSSLEVDRRASSSRFSSTNYSDNYDNYDEEEIDRTIRAIIGRCKDHVCKTNKKKVNGMKSISFLLKKMFVCSSGFAPTPSLRDTFPESRMEKLLRTILSKKIINPQNAARVSTKRYLEDRCVPKEEEEEKKREKTCDGSKWVKTDSD

SEQ ID NO: 40 - DNA, 837 bp, Nicotiana attenuata
ttgcagttctttagctggatgcaaaataagttcaatggcggacaagggaacagatcaatgcctaatgaagttcaaaccaaaaaacgtcctcgcaacgaagaattcaacggttggcctgattcgttattagccattggaacttttggtaccagcagcagtaatctcaaagcaaaatcagagagccaaaacgtacaaaatcaagaacgggatgaaataatcttagatgataatattaatgagcaaagttcctctccagatttagcagaattcacacctgaagaagttggtaaattacagaaagaattaacaaagttattatcaaaaaaaccagctgctaaattaattgatcaaggacgacaagatggtgatctcccattggatagattccttaattgcccttcaagtttagaagtggatcgtagggcttcttccagcagatttagcagtactaattactcagataattatgataattatgatgaggaagaaattgatagaactattagagcaatcattggaagatgcaaggatcatgtttgcaagacaaataaaaagaaagtaaatgggatgaaatccatttctttccttctcaagaaaatgtttgtttgctcaagtggttttgctcctactcctagtttacgagatacatttccagaatcaagaatggagaagcttttaaggacaatactttccaagaaaataataaaccctcaaaatgcagctcgagtatcaacaaagagatacttagaggaccgatgtgtaccaaaggaagaggaagaggagaaaaaacgggagaaaacttgtgatggatctaagtgggtgaagactgattctgat

SEQ ID NO: 41 - protein, 281 aa, Nicotiana attenuata
CPQITNFANVNSRFILDMKFFNWMHNKLNGGQGSKKPNAVPITNQTNEEFKDWPDSLLAIGTFGNKSSDLEESRPKTHVQNDHHHEDEILENSPDLAEFTPEEVGKLQKELTKLLSRKPADDILPLDRFLNCPSSLEVDRRISSSSTNSDNFDYDEEEIDRTIRVIIGRCKDVCSKQNKKKAIGKKSISFLLKKMFACASGNFGPPPTFPDPFHESRMEKLLRTMLSKKINPQNASRTSTKRYLEDKQPKKEEQEEKKREKTCNDGSKWVKTDSEFIVLEM

SEQ ID NO: 42 - DNA, 846 bp, Nicotiana attenuata
tgtccacaaattaccaacttcgcaaacgtcaacagcagattcattttagatatgaagttctttaactggatgcataataagttaaatgggggacaaggaagcaaaaaacctaatgcagttcctatcacaaatcaaacaaatgaagagtttaaagattggccagattcgttattggcaattggaacttttggcaacaagagcagtgatctcgaagaaagtagaccaaaaacacacgtacaaaatgatcatcatcacgaggacgaaatcctagagaattcaccagatttagcagaattcacacctgaagaagttggcaaattacaaaaagaattaacaaaattattatcccgaaaaccggctgatgatattcttccattggacagatttcttaattgtccgtcaagtttggaagttgatcgcaggattagttccagcagtactaattcagacaattttgattatgacgaggaagaaattgacagaactataagagtgattataggaagatgcaaagatgtctgtagtaagcagaacaaaaagaaagcaattgggaagaaatctatttcttttcttctcaagaaaatgttcgcttgtgcaagtggtaattttggtccacctcctactttcccagatccatttcacgaatcaagaatggagaagcttttgaggacaatgctttccaagaaaataaaccctcaaaatgcctctcggacatcaacaaagagatatttagaggacaaacaaccaaaaaaggaagagcaagaagagaaaaaacgagagaaaacctgtaatgatggatctaaatgggtgaaaactgattctgaatttatcgtcttggagatgtag

SEQ ID NO: 43 - protein, 267 aa, Solanum lycopersicum
MKLFSWVQNKFNGGQVNKVQTKNQPSKEPRNEEFNGWPDSLLAIGTFGASSSSLKPKIQNDNDNDNEISEDVKQSSSPDLAEFTPEEVGKLQKELTKLLSKKPAAAAKLTAAAEGRQDGNLPLDRFLNCPSSLEVDRRTSSRFSSTNSEIYENLDEEEIDRTIRAIIGRLNGMKSVTFLLKKMFVCSSGFAPTPNLRDTLPESRMEKLLRTILSKKIIPQSASRISTKRYLEDRCVPKEEVEEKKRDKTCDGSKWVKTDSDFIVLEI

SEQ ID NO: 44 - DNA, 849 bp, Solanum lycopersicum
atgaagttctttaattggatgcataacaagctcaatggtggacaaggaagtaggaggtctaatgctatgccaattactacaaatcataatataaatgaagaattcaaagattggccagattcgttgttatcaattggaacttttggcaatagaagcagtgatctcaaagaacagagcaaattacacgtgaaagacgatgaactaacttcttattcttcttctccagaattagcagaattcacgtctgaagaagtcgagaagttacagaaggagttaacaaagttactatcacgaaaaccacccccaactgctagtaattctgagtttgttgacatcaagaacggcgctgccaatgctgatgatatccttccgttggacagatttcttaattgtccatcgagcttggaagttgatcgtagggttaattccagtagatttagcagtgttaattactcgtacgattacgacgaggaagaaatcgacagaacaataagagtaattataggtagatgcaaggatgtttgtagaaaacagagcaaaaagaaatcaattgggatgaaatcaatttctttccttctcaagaaaatgcttgtttgtacaaagggtggttttgctcccgctcccaatttacgtgacacatttcccgaatcaagaatggagaagcttttgaggacaatgctttccaagaaaatacatccccaaaatgcccctcgaacatcaacaaagagatatttagaggaaaaacatgcacaaagagaagagaaagaagagaaaaaaagagaggaaaatagttatgatggatctaaatgggtgaagactgattctgaatttatcgtcttggaaatatag

SEQ ID NO: 45 - DNA, 20 bp, Artificial Sequence
  gRNA for wheat and barley
tcgaccggcggctctcgctc

SEQ ID NO: 46 - DNA, 20 bp, Artificial Sequence
  gRNA for Zea Mays
gcctcgaggtcgaccggagg

SEQ ID NO: 47 - DNA, 20 bp, Artificial Sequence
  gRNA for Glycine max
cttcaagcttggaggttgat

SEQ ID NO: 48 - DNA, 20 bp, Artificial Sequence
  gRNA for Brassica rapa
tcgagtcttgaagtcgatag

SEQ ID NO: 49 - DNA, 20 bp, Artificial Sequence
  gRNA for Brassica rapa
tcgagtcttgaagtcgacag

SEQ ID NO: 50 - DNA, 20 bp, Artificial Sequence
  gRNA for Oryza sativa
gcctcgaggtcgaccggcgc

SEQ ID NO: 51 - DNA, 20 bp, Artificial Sequence
  gRNA for Oryza sativa
gcctcgaggtggacaggaga

SEQ ID NO: 52 - DNA, 20 bp, Artificial Sequence
  gRNA for Sorghum bicolor
tcgaccggaggatctccctg

SEQ ID NO: 53 - DNA, 20 bp, Artificial Sequence
  gRNA for Sorghum bicolor
gcctggaggtggacaggcgg

SEQ ID NO: 54 - DNA, 20 bp, Artificial Sequence
  gRNA for Gossypium raimondii
catcgagcttggaagttgat

SEQ ID NO: 55 - DNA, 20 bp, Artificial Sequence
  gRNA for Gossypium raimondii
tcaagcttggaagtggatag

SEQ ID NO: 56 - DNA, 19 bp, Artificial Sequence
  gRNA for Nicotiana attenuate
caagtttagaagtggatcg

SEQ ID NO: 57 - DNA, 20 bp, Artificial Sequence
  gRNA for Nicotiana attenuate
tcaagtttggaagttgatcg

SEQ ID NO: 58 - DNA, 20 bp, Artificial Sequence
  gRNA for Solanum lycopersicum
tcgagcttggaagttgatcg

SEQ ID NO: 59 - DNA, 20 bp, Artificial Sequence
  gRNA for Brassica Oleracea
tcgagtcttgaagtcgatag

SEQ ID NO: 60 - DNA, 20 bp, Artificial Sequence
  gRNA for Brassica Oleracea
tcgagttttgaagtcgatag

SEQ ID NO: 61 - protein, 290 aa, Brassica Oleracea
MKLFGWMQNKLHGKQGNTHRPSTSSASSHQPREEFSDWPHGLLAIGTFGSVAKEQTPIETVQEEKPSNVHVEGQAQDRDQDLSPSGDLEDFTPEEVGKLQKELTKLLTRKNKKRKSDVNRELANLPLDRFLNCPSSLEVDRRISNALSGGGGDCDENEEDIERTISVILGRCKAISTESNSKKKKTKKDLSKTSVSYLLKKMFVCTEGFSPLPKPILRDTFQESRMEKLLRVMLLKKINAQAPSKETPMKKYVQDEQQLSLKNEEEEGSSSSSDGCKWVKTDSDFIVLEI

SEQ ID NO: 62 - DNA, 873 bp, Brassica Oleracea
atgaagctctttggatggatgcagaacaagctacatgggaaacaagggaacactcatagaccaagtacatcctctgcttcttctcatcaaccacgagaggagttcagcgactggcctcatggactacttgcgattggaacgttcggtagtgtggccaaagagcaaacaccaatagagactgttcaagaagagaagccctctaacgtgcacgtggaaggtcaagcgcaagatagagatcaagatctttcaccctccggtgacctagaagatttcactccggaggaagttgggaaacttcagaaggagctgacgaagctcttgacaagaaagaacaagaagaggaagtccgatgtgaatagagaacttgcgaatcttcctctggatagattcttgaattgtccttcgagtcttgaagtcgatagacgaatcagcaacgctctttctggtggtggtggagattgtgatgagaacgaagaagacattgagcgtacgatcagtgttatcttgggaagatgcaaagccatttctacagagagtaacagtaagaagaagaagactaagaaagatttgagcaaaacctctgtctcttatctcctcaagaagatgtttgtctgtacagaagggttctctcctcttcctaaacctatcttgagagacacgtttcaagaatcaagaatggaaaagttactgagggtgatgctactcaagaagattaatgctcaagctccctcgaaggaaacaccaatgaagaaatacgtgcaagacgagcaacagctttcactaaagaatgaggaagaagaaggaagtagtagtagtagcgatgggtgtaaatgggtcaaaacagattctgatttcattgttcttgagatctga

SEQ ID NO: 63 - protein, 288 aa, Brassica Oleracea
MKLFGWMQNKLHGKQGNTHRPSISSASSHQPREEFSDWPQGLLAIGTFGSVAKEQTQIQVVQEVFKEENPSDVNMEAHRDQDLSFSGDLDDFTPEEVGKLQKELTKLLTRKNKMRKSDVNRELANLPLDRFLNCPSSLEVDRRISNALASGGDFDENEEEMERTISVILGRCKAISTESSNKKKKSKRDLSKTSVFYLFKKMFVCSEGLSPLPNPSLRDTFQESRMEKLLRVMLHKKINAQASSKQTSTKRYVEDKQQLSLKNEEEEGRSGDGSKWVKTDSDFIVLEI

SEQ ID NO: 64 - DNA, 867 bp, Brassica oleracea
atgaagttattcggatggatgcagaacaagctacatgggaaacaagggaacactcatagaccaagcatatcttctgcttcttctcatcaacccagagaggagttcagcgactggcctcaaggattacttgcgattggaactttcggtagtgtggccaaagagcaaacacaaatacaagttgttcaagaagtgttcaaagaggagaatccctctgacgtgaacatggaagctcatagagatcaagatctttctttctccggtgatcttgatgattttactcccgaggaagtcgggaaactgcaaaaggaactgaccaagctcttgacaagaaagaacaagatgaggaagtctgatgtaaatagagaacttgcgaatcttcctttggatagattcttgaactgtccttcgagtcttgaagtcgatagacgaatcagcaacgcgctcgctagtggtggtgattttgatgagaacgaagaagaaatggagcgtacaatcagtgttatcttgggaagatgcaaagctatttctacagagagcagcaataaaaagaagaagagtaagagagatttgagcaaaacctctgttttttatcttttcaagaagatgtttgtatgttcagaggggttatctcctcttcccaaccctagcttgagagacacgtttcaagaatcaagaatggaaaagttactgagggtgatgctacacaagaagattaatgctcaagcttcctcgaagcaaacatcaacaaagagatacgtggaagataagcaacagctttcactaaagaacgaggaagaagaaggaagaagtggtgatgggagcaaatgggttaaaacagattctgatttcattgttcttgagatctga

SEQ ID NO: 65 - protein, 265 aa, Brassica oleracea
MHNKLHGKQANTHKRRTSSACSHQSREEFSDWPHGLLAIGTFGTLTKDQTPIQEVIQEEKTSNMHVEGRAQDRDHDISLSDDLEDFTPEEVGKLQNELTKLLTRKNKKRKSDVNKELANLPLDRFLNCPSSFEVDRRISNAFSGGGDSDENQEDIERTISIILGRCKAIYTESKNKKKGKRDVSKTSVSYLLKKMFFLRVMLLKKINTRASPKQTSTSRYVQDRQQLSLKNKEEEGRSSSSSDGSKWVKTDSDCSYRKVQIENLH

SEQ ID NO: 66 - DNA, 798 bp, Brassica oleracea
atgcataataagctacatggtaaacaagcgaatactcataaacgaagaacatcatctgcttgttctcatcaatcacgagaagagttcagcgattggcctcacggattacttgccattggaacgttcggtaccttgaccaaagatcaaaccccaatacaagaagtgattcaagaagagaagacttctaacatgcacgtggaaggtagagcgcaagatagagatcacgatatttctttatccgatgatcttgaagattttactcccgaggaagttgggaaactacaaaatgagctgacgaagctcttgacaagaaagaacaagaagaggaagtctgatgtgaacaaagaacttgccaatcttcctttggatagattcttgaattgtccttcgagttttgaagtcgatagacgaatcagcaacgcgttttcaggtggtggagattctgatgagaaccaagaagacattgagcgtacgattagtattattttggggagatgcaaagctatttatacagagagtaaaaataagaagaagggtaagagagatgtgagcaaaacctctgtttcttatctcctcaagaagatgttttttctgagagtaatgctactcaagaagattaatactcgagcttctccaaagcaaacatcaacgagtagatacgtgcaagacaggcaacaactttcattaaagaataaggaagaagaaggaagaagtagtagtagtagcgatgggagtaaatgggtcaaaacagattctgattgttcttacaggaaagtacagatagagaatcttcattga

SEQ ID NO: 67 - protein, 280 aa, Triticum aestivum
MGIINWVQNRLNTKQEKKRSAAAAAAGASSVRNAPVRENSCRGQADDELPGDWSMLSIGTIGTLGNEPTPAPAPDQAVPDFTIEEVKKLQDALNKLLRRAKSKSSSRGSTAGAGDEEQNLPLDRFLNCPSSLEVDRRLSLRLQGADGGQNGEFSPDTQIILSKARELLVSTNGNGGGVKQKSFKFLLKNMFACRGGFPPQPSLKDPVETKLEKLFKTMLQKKMSAPRQSNAASSSRKYYLEDKPMGRIQMDGHHDEEEDDYGEDVFKWDKTDSDFIVLEV

SEQ ID NO: 68 - DNA, 837 bp, Triticum aestivum
atcatcaactgggtgcagaatcgtctgaacaccaagcaggagaagaaacgatccgccgccgccgccgccgcgggcgcgagctcggttcgcaatgccccggtccgggagaatagttgccgcggccaggccgacgacgaactccccggcgactggagcatgctctccatcggaaccatcggaaccctcggcaacgagcccacgccggcgccggcgccagatcaggcggtgccggacttcaccatcgaggaggtgaagaagctgcaggacgcgctgaacaagctactcaggcgcgccaagtctaagtccagctcccgcggctccaccgccggcgccggcgacgaggagcagaacctgccgctcgacaggttcctcaactgcccctccagcctcgaggtcgaccggcggctctcgctcaggctgcagggcgccgatggcgggcagaacggggagttctcgccggacacgcagatcatactcagcaaggccagggagctcctcgtcagcaccaacggcaacggcgggggcgtcaagcagaagtccttcaagttcctcctcaagaacatgttcgcctgccggggcggcttcccgccgcagcccagcctcaaggatccagtcgaaacaaaactagagaagttgtttaagacgatgcttcaaaagaagatgagcgccccgcgccagagcaacgcggcatcgtcgtcgaggaagtattacctggaggacaaaccaatgggaaggatccaaatggatggtcaccacgacgaggaggaggatgactacggagaagatgtcttcaagtgggacaaaacagattcagatttcattgttctagaggtgtaa

SEQ ID NO: 69 - protein, 281 aa, Triticum aestivum
MGIINWVQNRLNTKQEKKRSAAAAAAGASSVRNAPVREKSCRGQADDELPGDWSMLSIGTLGNEPTPAPAPAPDQAVPDFTIEEVKKLQDALNKLLRRAKSKSSSRGSTAGAGDEEQNLPLDRFLNCPSSLEVDRRLSLRLQGADGGQNGEFSPDTQIILSKARELLVSTNGNGGGVKQKSFKFLLKNMFACRGGFPPQPSLKDPVETKLEKLFKTMLQKKMSVPRPSNAASSSRKYYLEDKPMGRIQMDGRHDEEEEEDYNDEDIFKWDKTDSDFIVLEV

SEQ ID NO: 70 - DNA, 846 bp, Triticum aestivum
atggggatcatcaactgggtgcagaatcgcctcaacaccaagcaggagaagaaacgatccgccgccgccgccgccgcgggcgcgagctcggttcgcaatgccccggtccgggagaagagctgccgcggccaggccgacgacgagctccccggagactggagcatgctctccatcgggactctcggcaacgagcccacgccggctccggcgccggcgccagatcaggcggtgccggacttcaccatcgaggaggtgaagaagctgcaggatgcgctgaacaagctactccggcgcgccaagtccaagtccagctcccgcggctccaccgccggcgccggcgacgaggagcagaacctgccgctcgacaggttcctcaactgcccctccagcctcgaggtcgaccggcggctctcgctcaggctgcagggcgccgacggcgggcagaacggggagttctcgccggacacgcagatcatactcagcaaggccagggagctcctcgtcagcaccaacggcaacggcgggggcgtcaagcagaagtccttcaagttcctcctcaagaacatgttcgcctgccggggcggcttcccgccgcagcccagcctcaaggatccagtggaaacaaaactggagaagttgtttaagacgatgcttcaaaagaagatgagcgtccctcgcccgagcaacgcggcatcgtcatcgaggaagtattacctagaggacaaaccaatgggaaggatccaaatggatggtcgccacgacgaggaggaggaagaggattacaatgatgaagatatcttcaagtgggacaaaacagattcagatttcattgttctagaggtgtaa

SEQ ID NO: 71 - protein, 276 aa, Triticum aestivum
MGIINWVQNRLNTKQEKKRSAAAAGASSVRNAPVREKSCRGQGDDELPGDWSMLSIGTLGNEPTPAPAPDQGVPDFTIEEVKKLQDALNKLLRRAKSKSSSRGSTAGAGDEEQNLPLDRFLNCPSSLEVDRRLSLRLQGADGGQNGEFSPDTQIILSKARELLVSTNGNGGGVKQNSFKFLLKNMFACRGGFPPQPSLKDPVETKLEKLFKTMLQKKMSAPRQSNAASSSRKYYLEDKPMGRIQMDGRHDEDEEDDYGEDVFKWDKTDSDFIVLEV

SEQ ID NO: 72 - DNA, 831 bp, Triticum aestivum
atggggatcatcaactgggtgcagaatcggctaaacaccaagcaggagaagaaacgatccgccgccgccgccggggcgagctcggttcgcaatgccccggtccgggagaagagctgccgcggccagggcgacgacgagctccccggcgactggagcatgctctccatcggaaccctcggcaacgaacccacgccggcgccggcgccagatcagggggtgccggacttcaccatcgaggaggtgaagaagctgcaggacgcgctgaacaagctactccggcgcgccaagtccaagtctagctcccgcggctccaccgccggcgccggcgacgaggagcagaacctgccgctcgacaggttcctcaactgcccctccagcctcgaggtcgaccggcggctctcgctcaggctgcagggcgccgatggcgggcagaacggggagttctcgccggatacgcagatcatactcagcaaggccagggagctcctcgtcagcaccaacggcaacggcgggggtgtcaagcagaattccttcaagttccttctcaagaacatgttcgcctgccggggcggcttcccgccgcagcccagcctcaaggatccagttgaaacaaaactggagaagttgtttaagacgatgcttcaaaagaagatgagcgccccgcgccagagcaacgcggcatcgtcgtcgaggaagtattacctagaggataaaccaatggggaggatccaaatggatggtcgccacgacgaggatgaggaggatgactatggagaagatgtcttcaagtgggacaaaacagattcagatttcattgttctagaggtgtag

SEQ ID NO: 73 - protein, 18 aa, Artificial Sequence
  LAZY4D motif
  misc_feature (12)..(12): Xaa can be S or C
LPLDRFLNCPSXLEVDRR